(19) United States
(12) Patent Application Publication
(10) Pub. No.: US 2025/0170224 A1
(43) Pub. Date: May 29, 2025

(54) METHODS AND COMPOSITIONS FOR TREATING MICROGLIAL DYSFUNCTION AND IMPROVING METABOLIC DYSFUNCTION

(71) Applicants: Ultimate Medicine AG, Zurich (CH); Albert-Ludwigs-Universitat Freiburg, Freiburg im Breisgau (DE)

(72) Inventors: Antal Szalay, Zurich (CH); Thomas Blank, Kirchzarten (DE)

(21) Appl. No.: 18/708,545

(22) PCT Filed: Nov. 8, 2022
(86) PCT No.: PCT/IB2022/000668

§ 371 Date: May 8, 2024

Related U.S. Application Data
(60) Provisional application No. 63/276,996, filed on Nov. 8, 2021.

(51) Int. Cl. A61K 38/46; A61K 31/37; A61P 25/28; G01N 33/68

(52) U.S. Cl. CPC A61K 38/465; A61K 31/37; A61P 25/28; C12Y 301/03001; G01N 33/6896; G01N 2800/2814; G01N 2800/50

(57) ABSTRACT

The present disclosure relates to methods and composition for treating or decreasing the rate of development of cognitive impairment, a neurodegen erative disease, or neuronal dysfunction in a. subject in need thereof. The methods include administration to the subject of a gut barrier function enhancer and/or an agent for reducing or eliminating gut microbiota dysfunction. The disclosure also provides methods for identifying such a subject.

### CROSS REFERENCE TO RELATED APPLICATIONS

This application claims the benefit of and priority to U.S. Provisional Application No. 63/276,996, filed Nov. 8, 2021, the entire disclosure of which is hereby incorporated by reference in its entirety for all purposes.

### FIELD

The present disclosure relates to methods and compositions for treating or decreasing the rate of development of a neurodegenerative disorder, cognitive impairment, or neuronal dysfunction in a subject.

### BACKGROUND

During normal brain aging, microglia often display a distinct transcriptional profile that is indicative of neurodegeneration (Salter et al. (2014) CELL, 158:15-24). At the same time, microglia in the aged human brain exhibit morphological changes and a reduced ability to support (Streit et al. (2004) GLIA 45: 208-212) other tissues (e.g., neurons), and become dystrophic. It has been suggested that age-related changes in microglia homeostasis are likely due to age-related changes in microglial homeostasis via the intrinsic and extrinsic factors. It remains to be understood what external factors mediate bidirectional interaction between the central nervous system (CNS) and the peripheral environment.

Despite the efforts that have been made to date there is still a need for new therapies for treating, or decreasing the rate of development of a neurodegenerative disorder, cognitive impairment, or neuronal dysfunction.

### SUMMARY

The present disclosure is based, in part, upon the discovery that the metabolite N⁶-carboxymethyllysine (CML), produced by gut microbiota and also present in processed food, can drive age-related oxidative stress and mitochondrial damage in microglia, wherein the amount of CML, which increases in the brain during aging can result in cognitive impairment and neurodegenerative disorders. Furthermore, it has been discovered that the increase in, or accumulation of, CML in body fluid and tissue samples during aging in a subject, can result from an increase in gut permeability as aging progresses, which is believed to result in higher levels of CML passing through the gut wall and into the body fluids and tissues of the subject. Based upon these discoveries, it is possible to provide therapies for treating, or decreasing the rate of development of a neurodegenerative disorder, cognitive impairment, or neuronal dysfunction.

In one aspect, the disclosure provides a method of decreasing the rate of development of oxidative stress or mitochondrial dysfunction in microglia, development of mitochondrial dysfunction in microglia, or microglial dysfunction in a subject in need thereof. The method comprises administering to the subject a therapeutically effective amount of a gut barrier function enhancer and/or an agent for reducing or eliminating gut microbiota dysbiosis.

In another aspect, the disclosure provides a method of treating cognitive impairment or decreasing the rate of development or worsening of cognitive impairment in a subject in need thereof. The method comprises administering to the subject a therapeutically effective amount of a gut barrier function enhancer and/or an agent for reducing or eliminating gut microbiota dysbiosis.

In another aspect, the disclosure provides a method of treating a neurodegenerative disease or decreasing the rate of development or progression of a neurodegenerative disease in a subject. The method comprises administering to the subject a therapeutically effective amount of a gut barrier function enhancer and/or an agent for reducing or eliminating gut microbiota dysbiosis.

In another aspect, the disclosure provides a method of decreasing the rate of development or worsening of neuronal dysfunction in a subject in need thereof. The method comprises administering to the subject a therapeutically effective amount of a gut barrier function enhancer and/or an agent for reducing or eliminating gut microbiota dysbiosis.

In each of the foregoing aspects, in one embodiment the subject has previously been identified as having an elevated level of N6-carboxymethyllysine (CML), a CML precursor, a CML metabolite (the terms CML metabolite and CML breakdown product are used interchangeably herein), or a CML analog in a biological sample of a subject as compared to a reference level. Alternatively or in addition, in another embodiment, the method further comprises identifying the subject as having an elevated level of CML, a CML precursor, a CML metabolite, or a CML analog in a biological sample of the subject as compared to a reference level. Depending upon the circumstances, the method further comprises determining the level of CML, a CML precursor, a CML metabolite, or a CML analog in the biological sample obtained from the subject. Furthermore, depending upon the circumstances, the biological sample comprises a body fluid (e.g., saliva, urine, blood, serum, plasma, cerebrospinal fluid, or feces) or tissue sample (e.g., brain tissue).

In each of the foregoing aspects and embodiments, (i) the subject has previously been identified as having an elevated level of permeability of the gut barrier as compared to a reference level; and/or (ii) the method further comprises identifying the subject as having an elevated level of permeability of the gut barrier as compared to a reference level.

In each of the foregoing aspects and embodiments, the subject has been identified or diagnosed as having: (i) a cognitive impairment; and/or (ii) a neurodegenerative disease. Alternatively or in addition, the subject has been identified as having an increased risk of developing: (i) a cognitive impairment; and/or (ii) a neurodegenerative disease.

In certain embodiments, the neurodegenerative disease is selected from the group consisting of: Alzheimer's disease, Parkinson's disease, Huntington disease, frontotemporal dementia, amyotrophic lateral sclerosis, multiple sclerosis, glaucoma, myotonic dystrophy, progressive supranuclear palsy, spinal muscular atrophy, multisystem atrophy, ataxias, and vascular dementia.

In each of the foregoing aspects and embodiments, depending upon the circumstances, the method results in one or more of (i) a reduction in level of cellular and/or mitochondrial reactive oxidative species (ROS) in microglia in the subject, (ii) a reduction in expression of inducible nitric oxide synthase (iNOS) in microglia in the subject, (iii) a reduction in expression of one or more genes in microglia of the subject selected from the group consisting of Cdkn1a, Cyba, Cybb, Duoxa1, Il1b, Tgehr2, Tlr2, Tlr4, Tlr5, Axl, Hif1a, Lcn2, Mmp2, Rela, Trex1, S100a8, and S100a9, and (iv) an increase in expression of one or more genes in microglia of the subject selected from the group consisting of Foxp1, Nrf1, Trp53, G6pdx, Pdk2, Sat3, and Ucp2.

In another aspect, the disclosure provides a method of decreasing the rate of accumulation of CML, a CML precursor, a CML metabolite, or a CML analog in a tissue of a subject. The method comprises administering to the subject a therapeutically effective amount of a gut barrier function enhancer and/or an agent for reducing or eliminating gut microbiota dysbiosis.

In another aspect, the disclosure provides a method of identifying a subject as having an increased risk of (i) developing microglial dysfunction, (ii) cognitive impairment, or (iii) developing a neurodegenerative disease. The method comprises identifying a subject having an elevated level of CML, a CML precursor, a CML metabolite, or a CML analog in a biological sample obtained from the subject as compared to a reference level.

In another aspect, the disclosure provides a method of reducing a concentration of CML, a CML precursor, a CML metabolite, or a CML analog in a blood or a brain sample of a subject in need thereof. The method comprises administering to the subject a therapeutically effective amount of a gut barrier function enhancer and/or an agent for reducing or eliminating gut microbiota dysbiosis.

In another aspect, the disclosure provides a method of reducing a concentration of CML, a CML precursor, a CML metabolite, or a CML analog in a blood or a brain sample to prevent or treat cognitive impairment or a neurodegenerative disease in a subject in need thereof. The method comprises administering to the subject a therapeutically effective amount of a gut barrier function enhancer and/or an agent for reducing or eliminating gut microbiota dysbiosis.

In another aspect, the disclosure provides a method of reducing gut permeability to prevent or treat cognitive impairment or a neurodegenerative disease in a subject in need thereof. The method comprises administering to the subject a therapeutically effective amount of a gut barrier function enhancer and/or an agent for reducing or eliminating gut microbiota dysbiosis.

In each of the foregoing aspects and embodiments, the gut barrier function enhancer comprises intestinal alkaline phosphatase (IAP), a polyphenol (e.g., ellagic acid (EA) and lipoteichoic acid), metformin, urolithin A, butyrate, glutamine, obeticholic acid (OCA), divertin, curcumin, spermidine, glutamine, or AMP-activated protein kinase (AMPK), or derivatives thereof. Similarly, in each of the foregoing aspects and embodiments, the agent for reducing or eliminating gut microbiota dysbiosis comprises intestinal alkaline phosphatase (IAP), ellagic acid (EA), a biotic, probiotic, prebiotic, or postbiotic. In certain preferred embodiments, the gut barrier function enhancer and/or an agent for reducing or eliminating gut microbiota dysbiosis is IAP. Similarly, in certain preferred embodiments, the gut barrier function enhancer and/or an agent for reducing or eliminating gut microbiota dysbiosis is EA. In each of the foregoing aspects and embodiments, the gut barrier function enhancer and/or an agent for reducing or eliminating gut microbiota dysbiosis is formulated as a pharmaceutical composition.

Depending upon the circumstances, the gut barrier function enhancer and/or an agent for reducing or eliminating gut microbiota dysbiosis is administered by oral administration, transdermal administration, inhalation, nasal administration, topical administration, intravenous administration, intra-arterial administration, intramuscular administration, or subcutaneous administration.

Upon administration of the gut barrier function enhancer and/or an agent for reducing or eliminating gut microbiota dysbiosis, the subject exhibits one or more of: (a) a reduced concentration of CML, a CML precursor, a CML metabolite, or a CML analog in a blood sample; (b) a reduced concentration of CML, a CML precursor, a CML metabolite, or a CML analog in a brain tissue sample; (c) a reduced permeability of the gut, (d) a reduction of microbiota dysbiosis; (e) an increased level of autophagy in gut epithelium; (f) a reduction in level of cellular and/or mitochondrial ROS in microglia; (g) an increased level of adenosine triphosphate (ATP) in a population of microglia; (h) a reduction in expression of iNOS in microglia; (i) a reduction in expression of one or more genes in microglia selected from the group consisting of Cdkn1a, Cyba, Cybb, Duoxa1, Il1b, Tgfbr2, Tlr2, Tlr4, Tlr5, Axl, Hif1a, Lcn2, Mmp2, Rela, Trex1, S100a8, and S100a9; and/or (j) an increase in expression of one or more genes in microglia selected from the group consisting of Foxp1, Nrf1, Trp53, G6pdx, Pdk2, Stat3, and Ucp2.

In another aspect, the disclosure provides, a method of identifying a subject as having an increased risk of developing microglial dysfunction. The method comprises identifying a subject having an elevated level of CML, a CML precursor, a CML metabolite, or a CML analog in a biological sample obtained from the subject as compared to a reference level, wherein such elevated level is indicative the subject has an increased risk of developing microglial dysfunction.

In another aspect, the disclosure provides a method of identifying a subject as having an increased risk of cognitive impairment. The method comprises identifying a subject having an elevated level of CML, a CML precursor, a CML metabolite, or a CML analog in a biological sample obtained from the subject as compared to a reference level, wherein such elevated level is indicative the subject has an increased risk of developing cognitive impairment.

In another aspect, the disclosure provides a method of identifying a subject as having an increased risk of developing a neurodegenerative disease. The method comprises identifying a subject having an elevated level of CML, a CML precursor, a CML metabolite, or a CML analog in a biological sample obtained from the subject as compared to a reference level, wherein such elevated level is indicative the subject has an increased risk of developing neurodegenerative disease.

In each of the foregoing aspects, the biological sample is a tissue (e.g., brain tissue) or body fluid sample (e.g., saliva, urine, blood, serum, plasma, cerebrospinal fluid, or feces). It is contemplated that the amount of CML, a CML precursor, a CML metabolite, or a CML analog in the biological sample can be measured by analytical techniques known in the art including for example, chromatography (e.g., high performance liquid chromatography (HPLC)), mass-spectroscopy, liquid chromatography-mass spectrometry (LCMS), nuclear magnetic resonance spectroscopy, or immunoassay.

### BRIEF DESCRIPTION OF THE DRAWINGS

FIGS. 1a-1g depict schematics and a set of graphs showing the interaction of microbiota on microglial transcriptome in young and aged mice. FIG. 1a is schematic representation of gut microbiota-mediated CML accumulation in the aged brain of a subject. CML induces aging features in microglia, specifically oxidative stress and mitochondrial damage. Rejuvenating the gut-blood barrier integrity limits CML accumulation and its detrimental effects on microglia.

FIG. 1b is a schematic diagram of the experimental approach where RNA sequencing (RNA-seq) is performed on fluorescence-activated cell sorting (FACS)-isolated microglia from the whole brain of young-adult (6-10-week-old) and aged (96-104-week-old) mice grown in specific pathogen-free (SPF) and germ-free (GF) environments.

FIG. 1c is a principal component analysis (PCA) on transcriptome (normalized gene counts) of microglia isolated from young-adult and aged SPF (n=6, 16) and GF (n=6, 8) mice.

FIG. 1d is a bar chart showing the number of upregulated and downregulated differentially expressed genes (DEGs) in GF versus SPF mice across the age groups. FIG. 1e is a heatmap of a subset of DEGs in GF versus SPF mice independent of age (the microglial GF signature). The list of genes with symbols (left) indicates their functional annotation (top left). Each column is a biological replicate and each row is a gene. DEGs (Wald P_{adj}<0.05 and absolute fold change>1.5). z-scores were calculated from normalized gene counts (upregulation in hashed gray, downregulation in unhashed gray).

FIG. if is a module-trait correlation across ages. Each subplot represents a different group with the depiction of all module eigengenes (MEs) extracted by weighted gene coexpression network analysis (WGCNA) (grayscale depicts correlation coefficient; radius: scaled −log₁₀(P_{adj})).

FIG. 1g depicts the significant Gene Ontology (GO) terms enriched in modules with the respective −log₁₀(P_{adj}). The number of genes per module (ME1-ME10) are shown. Statistics: FIGS. 1f-1g, Two-sided P values were obtained by Wald test and corrected for multiple testing using the Benjamini-Hochberg method.

FIGS. 2a-2i are a set of graphs and micrographs, respectively, showing that microbiota contribute to age-related oxidative stress and mitochondrial dysfunction in microglia.

FIG. 2a is a graph showing reactive oxygen species (ROS)-associated MEs (top to bottom: ME1, ME2, ME8) (grayscale: correlation coefficient; diameter: scaled −log₁₀(P_{adj})).

FIG. 2b is a heatmap of ROS-related genes in MEs (1, 2, 8) in the microglia of young-adult and aged SPF and GF mice. Each column is a biological replicate. The genes listed on the right hand side of the heat map are listed in order from top to bottom in the text following the heatmap.

FIG. 2c is a bar chart showing the quantification of cellular ROS relative to young-adult SPF mice. Data are presented as mean values+s.e.m. from 3 independent experiments including SPF (n=18, 14) and GF (n=13, 10).

FIG. 2d is a bar chart showing the quantification of iNOS⁺ Iba-1⁺ area in microglia relative to young-adult SPF mice. Data are presented as mean values+s.e.m. from two experiments and include SPF (n=14, 9) and GF (n=10, 9).

FIG. 2e are images showing the immunofluorescence of Iba-1, iNOS, and DAPI in the cortex of aging SPF and GF mice. Scale bar, 40 μm.

FIG. 2f shows metabolism-associated ME10 (grayscale: correlation coefficient; diameter: scaled log₁₀(P_{adj})).

FIG. 2g is a bar chart showing the percentage of healthy versus abnormal mitochondria in the cortical microglia of aged SPF and GF mice. Each dot represents an average of 30-35 cells from 1 mouse. Data are presented as mean values+s.e.m. from two independent experiments including aged SPF and GF mice (n=8 each). Two-way ANOVA followed by Sidak's multiple comparisons test (*P<0.05, **P<0.01, ***P<0.001; NS, not significant). Exact P values are reported in the Figure.

FIG. 2h is a set of electron micrographs of microglia from aged SPF and GF mice. Gray arrowheads, healthy; white arrowhead: abnormal. Scale bar—2 μm. Magnified micrographs of mitochondrial morphologies. Scale bar—500 nm.

FIG. 2i is a bar chart showing mitochondrial activity in microglia relative to young-adult SPF mice. Data are presented as mean values+s.e.m. from 3 experiments including SPF (n=17, 14) and GF (n=9, 13). Each dot represents one mouse. Statistics: FIGS. 2c, 2d, and 2i, Two-way ANOVA followed by Tukey's post-hoc test.

FIGS. 3a-3g are a set of graphs and charts showing microbiota- and age-associated regulation of serum and brain metabolites. FIG. 3a is a bar chart showing short-chain fatty acids (SCFAs) concentration in serum samples. Each dot represents one mouse. Data presented as mean values+s.e.m. from 1 experiment including young-adult and aged SPF mice (n=5, 6). Two-sided Mann-Whitney U-test.

FIG. 3b is a set of volcano plots of differentially abundant metabolites from non-targeted metabolomics analysis of serum (top: n=5, 6) and brain tissue (bottom: n=5, 5) samples of young-adult and aged SPF mice. X axis microbiota-gut: fold change. y axis: log₁₀(P).

FIG. 3c is a Venn diagram showing differentially abundant metabolites from serum (106) and brain (164) samples; and the overlap/intersect (19).

FIG. 3d is a chart showing metabolites that were differentially abundant in aging for both serum and brain specimens when compared to young mice. Biochemical name: the asterisk indicates a compound that has not been confirmed based on a standard. Bars to the left of zero on the x-axis indicate metabolites that were downregulated in aged mice when compared to young mice, while bars to the right of zero on the x-axis indicate metabolites that were upregulated in aged mice when compared to young mice.

FIGS. 3e-3f are a set of dot plots of CML (FIG. 3e) and TMAO (FIG. 3f), respectively, quantified by nontargeted metabolomics on serum in a human aging cohort (when detectable) from the TwinsUK data bank. Data are presented from Long et al. (2017) NAT. GENET., 49: 568-578 (n=6,194). Center line: best-fit value of the slope and intercept and error bars (95% confidence intervals (CIs)). a.u., arbitrary unit. Two-sided Pearson correlation analysis (* P<0.05, **P<0.01). Exact P values are reported in the Figure.

FIG. 3g is a heatmap depicting a subset of metabolites by targeted metabolomics on the brain of young-adult and aged SPF and GF mice (young-adult mice, n=5; aged mice, n=8 each). Each column represents data from one animal and each row represents a metabolite.

FIGS. 4a-4l is a schematic and, a set of graphs, and photomicrographs, respectively, showing that CML contributes to microbiota-mediated microglial aging. FIG. 4a is a schematic of metabolite treatment where young-adult SPF mice treated intraperitoneally with CML, TMAO, sodium acetate or sodium propionate daily for two weeks. FIG. 4b is a bar chart showing the quantification of ROS. FIG. 4c is a bar chart showing mitochondrial activity. FIG. 4d is a bar chart showing ATP levels. In FIGS. 4b-4d each dot represents one mouse and was depicted relative to vehicle-treated mice (n=4). Data are presented as mean values+s.e.m.

FIG. 4e is a bar chart showing the quantification of CML by targeted metabolomics in the brain: groups are as shown in FIG. 3g. Data are presented as mean values+s.e.m. from SPF and GF mice (young-adult, n=5; aged, n=8 each). FIG. 4f is a bar chart showing the brain of vehicle-treated or CML-injected young-adult SPF mice (n=5).

FIG. 4g is a heatmap depicting DEGs in the microglia of CML versus vehicle-injected mice. Each column is a biological replicate and each row is a gene. DEGs (Wald test P_{adj}<0.05 and absolute fold change>1.5). Upregulation in hashed gray and downregulation in unhashed gray. FIG. 4h is a volcano plot of DEGs in SPF versus GF microglia from aged mice (dots) with CML-specific genes (labeled).

FIG. 4i are immunofluorescence images of CML, Iba-1, and DAPI in the mouse cortex of young-adult and aged SPF and GF mice. Scale bars—50 μm (overview) and 10 μm (inset).

FIG. 4j is a graph showing the percentage of CML+Iba-1⁺ cells in the mouse cortex of young-adult and aged SPF (n=9, 8) and GF (n=9, 9) mice. Data are presented as mean values+s.e.m.

FIG. 4k is a graph showing the linear regression between the percentage of CML⁺ Iba-1⁺ cells and age in the human cortex. Each dot represents 1 individual (n=43; Pearson correlation analysis: r=0.5793, R2=0.3356, P<0.001). Males, n=23, light gray. Females, n=20, dark gray. Age, 1-88 years. Center line: Best-fit value of the slope and intercept. Dark gray line: 95% Cis.

FIG. 4l are immunofluorescence images of CML, Iba-1, and DAPI in the human cortex. Scale bars—50 μm (overview) and 10 μm (inset). White dashed boxes denote cell bodies. Statistics: for FIG. 4f, Two-sided Mann-Whitney U-test. For FIG. 4k, Two-sided Pearson correlation analysis. For FIGS. 4b-4d, One-way ANOVA followed by Dunnett's post-hoc test. For FIGS. 4e,4j, Two-way ANOVA followed by Tukey's post-hoc test (*P<0.05, **P<0.01, ***P<0.001), where P values are reported in the Figure.

FIGS. 5a-5j are a set of bar charts and a schematic, respectively, showing that the disruption of gut-blood barrier in aging instigates the CML surge. FIG. 5a is a bar chart showing the quantification of CML by targeted metabolomics (LC-MS) in fecal pellets freshly obtained from aged SPF and GF mice (n=5). FIGS. 5b-5c, are a set of bar charts showing the intestinal permeability measured by the percentage of fluorescent FITC-dextran (4 kDa) translocation to the circulation after oral gavage in young-adult and aged mice housed under SPF (n=5, 5) or GF (n=9, 4) conditions (FIG. 5b) and young-adult GF mice that received young or aged fecal microbiota transplantation (FMT) (n=8) (FIG. 5c). FIG. 5d is a bar chart showing the difference in CML translocated into the circulation 4 hours post-oral gavage in young-adult and aged mice housed under SPF or GF (n=5) conditions.

FIG. 5e is a schematic diagram: 18-month-old SPF mice were treated orally every third day for 10 weeks with vehicle (20% hydroxypropyl-p-cyclodextrin in 1×PBS), EA or IAP (n=4). FIG. 5f is a bar chart showing the intestinal permeability measured by the percentage of fluorescent FITC-dextran (4 kDa) translocation to the circulation after oral gavage. FIG. 5g is a bar chart showing the quantification of CML by targeted metabolomics (LC-MS) in the brain. FIG. 5h is a bar chart showing the quantification of relative mean fluorescence intensity of the CellROX probe signal. FIG. 5i is a bar chart showing the quantification of relative cellular ATP. Statistics: for FIGS. 5a,5c, Two-sided Mann-Whitney UI-test. For FIGS. 5f-5i, One-way ANOVA followed by Dunnett's post-hoc test. For FIGS. 5b,5d, Two-way ANOVA followed by Tukey's post-hoc test (*P<0.05, **P<0.01, ***P<0.001). P values are reported in the Figure. Data are presented as mean values+s.e.m.

FIGS. 6a-6h are a set of images and bar charts showing that the microbiota drives age-related differences in microglial morphology but not in cell density. FIG. 6a includes images showing the immunohistological detection of Iba-1⁺ microglia in the cortex of young-adult and aged SPF and GF mice. Scale bar—20 μm.

FIG. 6b is a bar chart summarizing microglia densities in the cortex. SPF (n=9, 8) and GF (n=9, 8).

FIG. 6c is a representative three-dimensional reconstruction of cortical microglia of all groups. Scale bar—10 μm.

FIGS. 6d-6h are bar charts showing an imaris-based semi-automatic quantification of cell morphology, where each chart shows the total branch length (gm; FIG. 6d), total branch area (μm²; FIG. 6e), number of branch points (FIG. 6f), cell body volume (μm³; FIG. 6g), and cell body sphericity (FIG. 6h), respectively. Each symbol represents an average of at least four cells measured per mouse. Data represent two independent experiments including young-adult and aged mice. SPF (n=8, 8) and GF (n=8, 8). Statistical analysis: FIGS. 6b-6h two-way ANOVA followed by Tukey's post-hoc test (*p<0.05, **p<0.01, ***p<0.001, ns=not significant). Data are presented as mean values+SEM. P-values are reported in the Figure.

FIGS. 7a-7b are a set of charts showing the gating strategy for flow cytometry and purity of MACS separation. FIG. 7a is a set of charts showing the cell sorting strategy for RT-qPCR and RNA-sequencing, where (1) shows results when myeloid cells were gated by size and granularity, (2 and 3) show results when only single cells were included, and (4) shows results when live and lineage cells were gated negative for Fixable Viability Dye eFluor® 780 and CD3, CD19, CD45R, Ly6C, and Ly6G to exclude T cells, B cells, monocytes and granulocytes, respectively, and (5) shows results when microglia were gated on CD45^{lnt} and CD11b⁺.

FIG. 7b are charts showing the purity of cells used for cellular ATP assay. Microglial cells were separated by percoll separation (insert 1), and enriched using the CD11b MACS cell separation system (Miltenyi Biotec, USA; insert 2), and the final results are depicted (insert 3), where each dot represents one mouse. Data are presented as mean values+/−SEM.

FIGS. 8a-8d are heatmaps showing the microglial transcriptional profile from GF and SPF mice of both sexes.

FIG. 8a is a heatmap of genes (normalized gene counts) specific to different types of immune cells in order to show purity of sorted cells. FIG. 8b is a heatmap showing sample-to-sample Ward clustering. FIG. 8c is a heatmap of all genes in the modules eigengenes. Each row is a biological replicate.

FIG. 8d is a heatmap of genes in metabolism-associated module eigengene ME10. Z-scores were calculated from normalized counts. Each row is a gene, and each column is a biological replicate: microglia isolated from young-adult and aged SPF (n=6, 16) and GF (n=6, 8) mice. The genes set forth on the right hand side of the heat map, are reproduced in the text consecutively from top to bottom in the text following the heatmap.

FIGS. 9a-9h are a set of micrographs and bar charts, respectively, showing age-related mitochondrial physiology in microglia of male and female SPF and GF mice. FIG. 9a are representative electron micrographs of abnormal versus healthy mitochondria in cortical microglia. FIG. 9b is a bar chart showing the quantification of mitochondrial area per microglia. FIG. 9c is a bar chart showing the number of mitochondria per microglia. FIGS. 9b and 9c are a set of bar charts generated from aged SPF and GF mice (n=8). FIG. 9d is a bar chart showing the Hif1a mRNA expression in microglia based on RNA-seq analysis (normalized gene counts). FIG. 9e is a bar chart showing the Hif1a mRNA expression by RT-qPCR in microglia of young-adult and aged SPF (n=8, 8) and GF (n=7, 10) mice. FIG. 9f is a bar chart showing the mitochondrial mass (MitoTracker Green MFI) for young-adult and aged SPF and GF mice. FIG. 9g is a bar chart showing the mitochondrial membrane potential (ΔΨm) (TMRM dye MFI) for young-adult and aged SPF and GF mice. FIG. 9h is a bar chart showing the quantification of cellular ATP for young-adult and aged SPF and GF mice males. Data were generated from young-adult and aged mice. Statistics: for FIGS. 9f and 9g SPF (n=17, 14) and GF (n=9, 13); for FIG. 9h SPF (n=23, 17) and GF (n=14, 11); for FIGS. 9b-9h data are presented as mean values+SEM. Statistical analysis for FIGS. 9b and 9c Mann-Whitney U test (two-sided), and for FIGS. 9e-9h two-way ANOVA followed by Tukey's post-hoc test (*p<0.05, **p<0.01, ***p<0.001, ns=not significant). P-values are reported in the Figure.

FIGS. 10a-10f are a set of graphs showing that CML modulates macrophage metabolism. FIGS. 10a-10b are charts showing pathway enrichment analysis for significantly abundant metabolites in serum (FIG. 10a) and brain (FIG. 10b) of aged mice (plotted are the top 15 enriched pathways). Color scale (black to lighter gray), ratio between the number of significant metabolites to the total number of metabolites detected in each pathway. Dot size reflects number of significant metabolites in each pathway. Pathway enrichment analysis was performed automatically using the Metabolon's client portal.

FIG. 10c is a bar chart showing the percentage of healthy versus abnormal mitochondria from total mitochondrial number in cortical microglia of young-adult mice treated with vehicle or CML i.p. (n=5).

FIGS. 10d and 10e are bar charts showing the cellular ROS (FIG. 10d) and mitochondrial activity (FIG. 10e), respectively, of bone marrow derived macrophages (BMDMs) that were cultured in serum-free medium 6 hours before the experiment. Cells were incubated with increasing concentrations of CML for 48 hours, before harvesting for measurements. Each dot is a biological replicate (n=3).

FIG. 10d is a bar chart showing the quantification of relative MFI of CellROX probe signals. FIG. 10e is a bar chart showing the mitochondrial activity depicted as mitochondrial membrane potential (ΔΨm) (TMRM dye MFI) normalized to mitochondrial mass (MitoTracker Green MFI). FIG. 10f is a principal component analysis (PCA) of the transcriptome (normalized gene counts) of microglia isolated from young-adult mice treated with vehicle or CML via intraperitoneal administration. Statistics for FIGS. 10c-10f: data are presented as mean values+SEM. Each dot represents one mouse. Statistical analysis: for FIG. 10c two-way ANOVA followed by Sidak's multiple comparisons test, for FIGS. 10d and 10e one-way ANOVA followed by Dunnett's post-hoc test (***p<0.001, ns=not significant). P-values are reported in the Figure.

FIGS. 11a-11f are a set of graphs showing the age-dependent shift in gut microbiota composition. FIG. 11a is a PCA plot (beta-diversity) and FIG. 11b is a Shannon and Simpson alpha-diversity plot of the indices of gut microbiota. To determine whether this was statistically significant, non-parametric Mann-Whitney U-tests (two-sided) were used to compare samples; Adonis (an analysis of variance using distance matrices) in vegan package was used to assess the effects of groups for beta diversity.

FIG. 11c is a graph showing the relative abundance of gut microbiota composition profiles at the phylum level in male mice at different ages (each color represents one bacterial phylum; the figure key is spatially arranged with the respective color representing a bacterial phylum located adjacent to its location on the graph). FIG. 1id is a bar chart showing the average Firmicules/Bacteroidetes ratio (F/R) in the fecal samples. FIG. 11e is a bar chart showing the relative abundance of the family Lachnospiraceae. FIG. 11f is a set of graphs showing the relative abundance of differentially abundant genera in aging. Taxonomic differences at phylum and genus levels between tested groups were identified using the “multivariate analysis by linear models” (MaAsLin) R package. Statistics: FIGS. 11a-11f are a set of graphs presenting data from young-adult and aged male mice, housed under SPF conditions (n=5, 10), where each dot represents data from one animal. FIGS. 11d and 11e present data as mean values+SEM. FIGS. 11b and 11f are a set of box plots, with the center line depicting the median and the upper and lower boundary of the box corresponding to the first and third quartiles (the 25th and 75th percentiles). The upper whisker extends from the hinge to the highest value that is within 1.5× the interquartile range (IQR) of the respective border, while the lower whisker extends from the respective border to the lowest value within 1.5× of the IQR of the border. IQR is the distance between the first and third quartiles. The statistics used in FIGS. 11d and 11e were the Mann-Whitney U test (two-sided).

FIGS. 12a-12e are a set of bar charts showing that age-related microglial CML accumulation is gut-mediated. FIG. 12a is a bar chart showing the targeted metabolomics (LC/MS) on CML translocated into the circulation 4 hours post oral gavage in young-adult and aged mice housed under SPF or GF (n=5). Light gray, before gavage, darker gray; 4 hours post gavage. Each dot represents an individual measurement for one mouse.

FIGS. 12b-12e are graphs and micrographs of data from young-adult and aged SPF mice injected with vehicle or CML (administered intraperitoneally (i.p.) or by oral gavage (o.g.)) (n=4 each). Each dot represents one mouse. FIG. 12b is a bar chart showing the percentage of CML⁺ Iba-1⁺ cells quantified in the cortex. FIG. 12c is an image of immunofluorescent labelling of CML, Iba-1, and DAPI in mouse cortex. Scale bars—50 μm (overview) and 10 μm (inset). FIG. 12d is a bar chart showing the quantification of relative cellular ROS probe signal by determining MFI. FIG. 12e is a graph showing the quantification of relative cellular ATP. Statistics: for FIGS. 12a, 12b, 12d, and 12e, each dot represents one mouse. Data are presented as mean values+SEM. Statistical analysis for FIGS. 12b, 12d, and 12e) two-way ANOVA followed by Tukey's post-hoc test (*p<0.05, **p<0.01, ***p<0.001, ns=not significant). P-values are reported in the Figure.

### DETAILED DESCRIPTION

### I. Definitions

As used in the specification and the appended claims, the singular forms “a,” “an” and “the” include plural referents unless the context clearly dictates otherwise. Thus, for example, reference to “a gut barrier function enhancer” can include mixtures of two or more such gut barrier function enhancers.

As used herein, the expression “and/or” in connection with two or more recited objects includes individually each of the recited objects and the various combinations of two or more of the recited objects, unless otherwise understood from the context and use. As used herein, unless specifically indicated otherwise, the word “or” is used in the inclusive sense of “and/or” and not the exclusive sense of “either/or.”

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

As used herein, the terms “administering” and “administration” refer to any method of providing an agent to the subject (e.g., a gut barrier function enhancer and/or an agent for reducing or eliminating gut microbiota dysbiosis). Such methods are known to those skilled in the art, and include, but are not limited to, oral administration, transdermal administration, administration by inhalation, nasal administration, topical administration, intravaginal administration, ophthalmic administration, intra-aural administration, intracerebral administration, administration to spinal cord, administration to intracerebral fluid, rectal administration, and parenteral administration, including injectable such as intravenous administration, intra-arterial administration, intramuscular administration, and subcutaneous administration. Administration can be continuous or intermittent. In some instances a gut barrier function enhancer and/or an agent for reducing or eliminating gut microbiota dysbiosis can be administered therapeutically. In other instances, a gut barrier function enhancer and/or an agent for reducing or eliminating gut microbiota dysbiosis can be administered prophylactically, such as administered for prevention of a disease or condition in a subject, or for improvement of one or more immune cell (e.g., microglia) functions in a subject (e.g., in the brain of a subject).

As used herein, the terms “effective amount” or “amount effective” or “therapeutically effective amount” refer to an amount that is sufficient to achieve the desired result (e.g., therapeutic benefit) or to have an effect on an undesired condition. For example, a “therapeutically effective amount” of a gut barrier function enhancer and/or an agent for reducing or eliminating gut microbiota dysbiosis may refer to an amount that is sufficient to achieve the desired result or to have an effect on a disease in a subject. Alternatively, or in addition, a “therapeutically effective amount” of a gut barrier function enhancer may refer to an amount of the gut barrier function enhancer for reducing or eliminating gut microbiota dysbiosis that is sufficient to reduce the level and/or activity of CML, a CML precursor, a CML metabolite, and/or a CML analog in a subject (e.g., in a biological sample obtained from the subject), or to improve one or more functions of an immune cell (e.g., microglia) in a subject (e.g., in the brain of a subject) to whom the gut barrier function enhancer and/or the agent for reducing or eliminating gut microbiota dysbiosis is administered. The specific therapeutically effective dose level for any particular subject will depend upon a variety of factors including: the disorder being treated and the severity of the disorder; the gut barrier function enhancer employed; the age, body weight, general health, sex, diet, ethnic group and/or geographical location of the subject; the time of administration; the route of administration; the rate of excretion of the specific gut barrier function enhancer employed; the duration of the treatment; drugs used in combination or coincidental with the specific gut barrier function enhancer employed and like factors known in the medical arts. For example, it is within the skill of the art to start doses of a therapeutic at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. In some instances, a gut barrier function enhancer and/or an agent for reducing or eliminating gut microbiota dysbiosis can be administered in a prophylactically effective amount.

As used herein with respect to a given parameter, the term “elevated level” refers to a level that is detectably higher (e.g., by about 5-10%, 10-20%, 20-30%, 30-40%, 40-50%, 50-60%, 60-70%, 70-80%, 80-90%, 85-95%, or more; such as, by about 5%, 10%, 15%, 20%, 25%, 30%, 35%, 40%, 45%, 50%, 55%, 60%, 65%, 70⁰/, 75%, 80%, 85%, 90%, 95%, 97%, 99%, or more) compared to a reference level. For example, as used herein, elevated level of CML, a CML precursor, a CML metabolite, or a CML analog in a subject may refer to detectably higher (e.g., by about 5-10%, 10-20%, 20-30%, 30-40%, 40-50%, 50-60%, 60-70%, 70-80%, 80-90%, or 85-95%, or more; such as, by about 5%, 10%, 15%, 20%, 25%, 30%, 35%, 40%, 45%, 50%, 55%, 60%, 65%, 70%, 75%, 80%, 85%, 90%, 95%, 97%, 99%, or more) level of CML, a CML precursor, a CML metabolite, or a CML analog compared to a reference level of CML, a CML precursor, a CML metabolite, or a CML analog. For example, an elevated level of one or more functions of an immune cell (e.g., microglia) in a subject (e.g., in the brain of a subject) may refer to detectably higher (e.g., by about 5-10%, 10-20%, 20-30%, 30-40%, 40-50%, 50-60%, 60-70%, 70-80%, 80-90%, or 85-95%, or more; such as, by about 5%, 10%, 15%, 20%, 25%, 30%, 35%, 40%, 45%, 50%, 55%, 60%, 65%, 70%, 75%, 80%, 85%, 90%, 95%, 97%, 99%, or more) level of one or more functions of an immune cell (e.g., microglia) in a subject (e.g., in the brain of a subject) as compared to a reference level of one or more functions of an immune cell (e.g., microglia) in a control subject (e.g., in the brain of a subject). In certain embodiments, an increased level of permeability of the gut barrier may refer to detectably higher (e.g., by about 5-10%, 10-20%, 20-30%, 30-40%, 40-50%, 50-60%, 60-70%, 70-80%, 80-90%, or 85-95%, or more; such as, by about 5%, 10%, 15%, 20%, 25%, 30%, 35%, 40%, 45%, 50%, 55%, 60%, 65%, 70%, 75%, 80%, 85%, 90%, 95%, 97%, 99%, or more) level of permeability of the gut barrier in a subject as compared to a reference level of permeability of the gut barrier in control subject.

In certain embodiments, an elevation or an increase can represent an increase by about 1% to about 300%, by about 1% to about 280%, by about 1% to about 260%, by about 1% to about 240%, by about 1% to about 220%, by about 1% to about 200%, by about 1% to about 180%, by about 1% to about 160%, by about 1% to about 140%, by about 1% to about 120%, by about 1% to about 100%, by about 10% to about 80%, by about 1% to about 60%, by about 1% to about 40%, by about 1% to about 20%, by about 20% to about 300%, by about 20% to about 280%, by about 20% to about 260%, by about 20% to about 240%, by about 20% to about 220%, by about 20% to about 200%, by about 20% to about 180%, by about 20% to about 160%, by about 20% to about 140%, by about 20% to about 120%, by about 20% to about 100%, by about 20% to about 80%, by about 20% to about 60%, by about 20% to about 40%, by about 40% to about 300%, by about 40% to about 280%, by about 40% to about 260%, by about 40% to about 240%, by about 40% to about 220%, by about 40% to about 200%, by about 40% to about 180%, by about 40% to about 160%, by about 40% to about 140%, by about 40% to about 120%, by about 40% to about 100%, by about 40% to about 80%, by about 40% to about 60%, by about 60% to about 300%, by about 60% to about 280%, by about 60% to about 260%, by about 60% to about 240%, by about 60% to about 220%, by about 60% to about 200%, by about 60% to about 180%, by about 60% to about 160%, by about 60% to about 140%, by about 60% to about 120%, by about 60% to about 100%, by about 60% to about 80%, by about 80% to about 300%, by about 80% to about 280%, by about 80% to about 260%, by about 80% to about 240%, by about 80% to about 220%, by about 80% to about 200%, by about 80% to about 180%, by about 80% to about 160%, by about 80% to about 140%, by about 80% to about 120%, by about 80% to about 100%, by about 100% to about 300%, by about 100% to about 280%, by about 100% to about 260%, by about 100% to about 240%, by about 100% to about 220%, by about 100% to about 200%, by about 100% to about 180%, by about 100% to about 160%, by about 100% to about 140%, by about 100% to about 120%, by about 120% to about 300%, by about 120% to about 280%, by about 120% to about 260%, by about 120% to about 240%, by about 120% to about 220%, by about 120% to about 200%, by about 120% to about 180%, by about 120% to about 160%, by about 120% to about 140%, by about 140% to about 300%, by about 140% to about 280%, by about 140% to about 260%, by about 140% to about 240%, by about 140% to about 220%, by about 140% to about 200%, by about 140% to about 180%, by about 140% to about 160%, by about 160% to about 300%, by about 160% to about 280%, by about 160% to about 260%, by about 160% to about 240%, by about 160% to about 220%, by about 160% to about 200%, by about 160% to about 180%, by about 180% to about 300%, by about 180% to about 280%, by about 180% to about 260%, by about 180% to about 240%, by about 180% to about 220%, by about 180% to about 200%, by about 200% to about 300%, by about 200% to about 280%, by about 200% to about 260%, by about 200% to about 240%, by about 200% to about 220%, by about 220% to about 300%, by about 220% to about 280%, by about 220% to about 260%, by about 220% to about 240%, by about 240% to about 300%, by about 240% to about 280%, by about 240% to about 260%, by about 260% to about 300%, by about 260% to about 280%, or by about 280% to about 300%) in a parameter or value as compared to a reference level.

As used herein, a “gut barrier function enhancer” refers to an agent that decreases, either directly or indirectly through an intermediate, the passage of CML, a CML precursor, a CML metabolite, and/or a CML analog from the subject's gut, and the rate of accumulation of CML, CML metabolite, a CML precursor, and/or a CML analog from the gut into a tissue or body fluid of a subject over time. Non-limiting examples of gut barrier function enhancers are intestinal alkaline phosphatase (IAP), a polyphenol (e.g., ellagic acid (EA) and lipoteichoic acid), metformin, urolithin A, butyrate, glutamine, obeticholic acid (OCA), divertin, curcumin, spermidine, glutamine, or AMP-activated protein kinase (AMPK), or derivatives thereof. For example, IAP is a gut barrier function enhancer that directly decreases the passage of CML, CML metabolite, a CML precursor, and/or a CML analog from the subject's gut and the rate of accumulation of CML, CML metabolite, a CML precursor, and/or a CML analog from the gut into a tissue or body fluid of a subject. EA is an example of a gut barrier function enhancer that indirectly (e.g., through an intermediate) decreases the passage of CML, CML metabolite, a CML precursor, and/or a CML analog from the subject's gut and the rate of accumulation of CML, CML metabolite, a CML precursor, and/or a CML analog from the gut into a tissue or body fluid of a subject. EA indirectly enhances gut barrier function because it is believed to reduce the expression of pore-forming claudin-4, -7 and -15 via Myosin Light Chain 2 (MLC2) signaling, where these claudins elicit leakiness of the gut.

As used herein, “gut microbiota dysbiosis” refers to an imbalance in the relative abundance or presence of microbes (e.g., beneficial and/or pathogenic microbes) in the gut of a subject that can result in a variety of symptoms including, for example, one or more of bloating, flatus, spasms, inflammation with loss of intestinal permeability, dysplasia of a mucosal surface, and insufficient reclamation of nutrients for buffering capacity. Dysbiosis can include a loss of beneficial microbes and/or an expansion of pathogenic microbes (e.g., pathobionts). Dysbiosis is believed to trigger pro-inflammatory effects and immune dysregulation associated with various disease states. As used herein, an “agent for reducing or eliminating gut microbiota dysbiosis” refers to agent that improves, either directly or indirectly through an intermediate, the dysregulation of gut microbiota that occur during dysbiosis. Non-limiting examples of an agent for reducing or eliminating gut microbiota dysbiosis (e.g., directly or indirectly) include, but are not limited to IAP and EA, biotics, prebiotics, probiotics and postbiotics. For example, probiotics cause a direct impact on the intestinal microbiome by the specific delivery of beneficial microbes to the gastrointestinal tract, such that probiotics are agents for directly reducing or eliminating gut microbiota dysbiosis. Exemplary probiotics include bacteria belonging to genera Lactobacillus, Bifidobacterium, and Streptococcus. Prebiotics enhance the growth of specific beneficial bacterial species that elicit health benefits, and exemplary prebiotics include lipoteichoic acid and a polyphenol. Postbiotics are, for example, metabolic products, fermentation products, minerals (e.g., zinc and selenium), microelements, micronutrients, cell surface proteins, and organic acids generated by the microbiome during its life cycle, and are characterized by products that provide a contribution to environmental eubiosis. Postbiotics indirectly shape the structure of the microbiota and are thereby agents for indirectly reducing or eliminating gut microbiota dysbiosis.

As used herein, the terms “neurodegenerative disease” or “neurodegenerative disorder,” which are used interchangeably, refer to one or more conditions from a heterogeneous group of disorders that are characterized by the progressive degeneration of the structure and/or function of the central nervous system or peripheral nervous system. Neurodegenerative diseases encompass a range of conditions that result from progressive damage to cells and nervous system connections that are essential for mobility, coordination, strength, sensation, and cognition. Common neurodegenerative diseases include, but are not limited to, Alzheimer's disease, Parkinson's disease, Huntington disease, frontotemporal dementia, amyotrophic lateral sclerosis, multiple sclerosis, glaucoma, myotonic dystrophy, progressive supranuclear palsy, spinal muscular atrophy, multisystem atrophy, ataxias, vascular dementia, or other dementias.

As used herein, the term “pharmaceutical composition” refers to the combination of an active agent (e.g., a gut barrier function enhancer and/or an agent for reducing or eliminating gut microbiota dysbiosis) with a carrier, inert or active, making the composition especially suitable for diagnostic or therapeutic use in vivo or ex vivo. As used herein, the term “pharmaceutical composition” can be a formulation containing a gut barrier function enhancer and/or an agent for reducing or eliminating gut microbiota dysbiosis of the present disclosure in a form suitable for administration to a subject. In one embodiment, the pharmaceutical composition is in bulk or in unit dosage form. The unit dosage form is any of a variety of forms, including, for example, a capsule, an intravenous bag, a tablet, a single pump on an aerosol inhaler or a vial. The quantity of active ingredient (e.g., a formulation of a disclosed gut barrier function enhancer and/or an agent for reducing or eliminating gut microbiota dysbiosis or salt, hydrate, solvate or isomer thereof) in a unit dose of composition is an effective amount and can vary depending upon the particular treatment involved. One skilled in the art will appreciate that it is sometimes necessary to make routine variations to the dosage depending on the age and condition of the subject. The dosage will also depend on the route of administration. A variety of routes are contemplated, including, for example, oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a gut barrier function enhancer include powders, sprays, ointments, pastes, creams, lotions, gels, solutions, patches and inhalants. In one embodiment, a gut barrier function enhancer is mixed under sterile conditions with a pharmaceutically acceptable carrier, and with any preservatives, buffers, or propellants that are required.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Adeboye Adejare, Remington: The Science and Practice of Pharmacy (23d ed. 2020). Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

The terms “pharmaceutically effective amount,” “pharmacologically effective amount,” “physiologically effective amount,” or “effective amount” of a gut barrier function enhancer and/or an agent for reducing or eliminating gut microbiota dysbiosis are used interchangeably and refer to the amount of a bioactive agent or combination of bioactive agents present in one or more pharmaceutical compositions as described herein that is needed to provide a desired level of active agent or agents in the bloodstream or at the site of action in an subject (e.g., the hepatic system, the renal system, the circulatory system, the lungs, the gastrointestinal system, the colorectal system, etc.) to be treated to give an anticipated physiological response when such composition is administered.

As used herein, the term “prevent” or “preventing” refer to precluding, averting, obviating, forestalling, stopping, or hindering something from happening, especially by advance action. It is understood that where reduce, inhibit or prevent are used herein, unless specifically indicated otherwise, the use of the other two words is also expressly contemplated. The term “prevent” does not require the 100% elimination of the possibility of an event. Rather, it denotes that the likelihood of the occurrence of the event has been reduced in the presence of a compound or method described herein. In various aspects, the terms cover any treatment of a subject, including a mammal (e.g., a human), and includes: (i) preventing the disease from occurring in a subject that can be predisposed to the disease but has not yet been diagnosed as having it; (ii) inhibiting the disease, such as arresting its development or decreasing the rate of its progress; or (iii) relieving the disease, such as causing regression of the disease.

As used herein with respect to a parameter or a rate, the term “reduce” or “reducing” or “decrease” or “decreasing” or “alleviate” or “alleviating” refers to a detectable change in the parameter or rate as compared to a control, such that the parameter or rate becomes smaller (e.g., by about 5-10%, 10-20%, 20-30%, 30-40%, 40-50%, 50-60%, 60-70%, 70-80%, 80-90%, or 85-95%; such as, by about 5%, 10%, 15%, 20%, 25%, 30%, 35%, 40%, 45%, 50%, 55%, 60%, 65%, 70%, 75%, 80%, 85%, 90%, 95%, 97%, 99%, or more) compared to the control.

As used herein, depending upon the context, a “control” refers to a sample that has not been exposed to a composition and/or method described herein or a control subject. A “control subject” refers to a subject who has not received a composition and/or method disclosed herein. As used herein, a “test subject” refers to a subject who has received or will receive the compositions and methods described herein. As used herein with reference to a parameter, a “suitable control” may refer to the parameter in a control subject (e.g., a test subject before receiving a treatment described herein; or a different subject, or group of subjects with like symptoms as a test subject, who did not receive the treatment described herein). For example, as used herein with reference to level of CML, a CML precursor, a CML metabolite, or a CML analog, a “suitable control” may refer to level of CML, CML analog, CML precursor, or CML metabolite in a subject (e.g., a test subject before receiving a treatment described herein; or a different subject, or group of subjects with like symptoms as the test subject, who did not receive the treatment described herein).

As used herein, with respect to a parameter, a “reference level” may refer to an established normal level of the parameter, or an established standard control. For example, as used herein with respect to level of CML, CML precursor, CML metabolite, or CML analog, a reference level may refer to the level of CML, CML precursor, CML metabolite, or CML analog in a subject or a group of subjects who do not show symptoms of a neurodegenerative disease, or cognitive impairment, and/or do not have an increased risk of developing a neurodegenerative disease or cognitive impairment.

The terms “subject,” “individual” and “patient” are used interchangeably and refer to an organism to be treated by the methods and/or compositions described herein. Such organisms preferably include, but are not limited to, mammals (e.g., murines, simians, equines, bovines, porcines, canines, felines, and the like), and more preferably include humans. The term does not denote a particular age or sex. Thus, adult and newborn subjects, whether male or female, are intended to be covered. For example, the subject can be a human. In particular, the subject can be a human with a neurodegenerative disease or at increased risk of developing a brain dysfunction, having impaired neuronal function, having a neurodegenerative disease (e.g., a subject previously identified or diagnosed as having a neurodegenerative disease) or a subject identified as having an increased risk of developing a neurodegenerative disease, or having cognitive impairment or a subject identified as having an increased risk of developing cognitive impairment.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “treatment” refers to the medical management of a subject with the intent to cure, ameliorate, stabilize, or prevent a disease or disorder. In certain embodiments, the term means an improvement in one or more immune cell (e.g., microglia) functions in a subject (e.g., in the brain of the subject). This term includes active treatment, causal treatment (e.g., treatment directed to the cause of the disease), palliative treatment (e.g., treatment designed for the relief of symptoms or complications associated with the disease), preventative treatment (e.g., treatment directed to delaying, minimizing, decreasing the rate of progression of, or partially or completely inhibiting the development or onset of the disease), and supportive treatment (e.g., treatment employed to supplement another therapy). Treatment also includes curing, suppressing, reducing, alleviating, and/or ameliorating one or more symptoms and/or complications associated with the disease. Treatment also includes prevention and/or decreasing the rate of progress of and/or delay of the onset of symptoms and/or complications associated with the disease. Treatment also includes diminishment of the extent of the disease; delaying or slowing or decreasing the rate of progress of the disease; amelioration or palliation of the disease; and remission (whether partial or total), whether detectable or undetectable. “Ameliorating” or “palliating” a disease means that the extent and/or undesirable clinical manifestations of the disease are lessened and/or time course or rate of the progression is slowed or lengthened, as compared to the extent or time course in the absence of treatment. Treatment does not require the complete amelioration of a symptom or complication associated with the disease, and encompasses embodiments in which one reduces symptoms and/or underlying risk factors of the disease. Those in need of treatment include those already with the disease, as well as those at a risk of having the disease or those in which the condition or disorder is to be prevented.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a method described herein can be combined in a variety of ways without departing from the spirit and scope of the present invention, whether explicit or implicit herein. For example, where reference is made to a particular compound, that compound can be used in various embodiments of compositions disclosed herein and/or in methods disclosed herein, unless otherwise understood from the context. In other words, within this application, embodiments have been described and depicted in a way that enables a clear and concise application to be written and drawn, but it is intended and will be appreciated that embodiments may be variously combined or separated without parting from the present teachings. For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### II. General Discoveries and Considerations

The present disclosure is based, in part, upon the discovery that the metabolite N⁶-carboxymethyllysine (CML), which can be produced by gut microbiota, can drive age-related oxidative stress and mitochondrial damage in microglia, wherein the amount of CML, which increases in the brain during aging can result in cognitive impairment, and neurodegenerative disorders. Furthermore, it has been discovered that the increase in, or accumulation of, CML in body fluid and tissue samples during aging in a subject, can result from an increase in gut permeability as aging progresses, which is believed to result in higher levels of CML passing through the gut wall and into the body fluids and tissues of the subject. Based upon these discoveries, it is possible to provide therapies for treating, or decreasing the rate of development of a neurodegenerative disorder, cognitive impairment, or neuronal dysfunction.

Although, microglial function declines during aging, it appears that, prior to this study, the interaction of microglia and the gut microbiota has not been well characterized during the aging process. As disclosed herein, the microglial transcriptomes from young-adult and aged mice housed under germ-free and specific pathogen-free conditions were compared, and it has been found that the microbiota influenced aging associated-changes in microglial gene expression. It was also found that the absence of gut microbiota diminished oxidative stress and ameliorated mitochondrial dysfunction in microglia from the brains of aged mice. Unbiased metabolomic analyses of serum and brain tissue revealed the accumulation of N⁶-carboxymethyllysine (CML) in the microglia of the aging brain. It appears that CML mediates a burst of reactive oxygen species and impedes mitochondrial activity and ATP reservoirs in microglia. Age-dependent rise in CML levels in the sera and brains of humans was also validated. In addition, microbiota-dependent increase in intestinal permeability in aged mice mediated the elevated levels of CML. The work described herein provides insight into how specific features of microglia from aged mice are regulated by the gut microbiota.

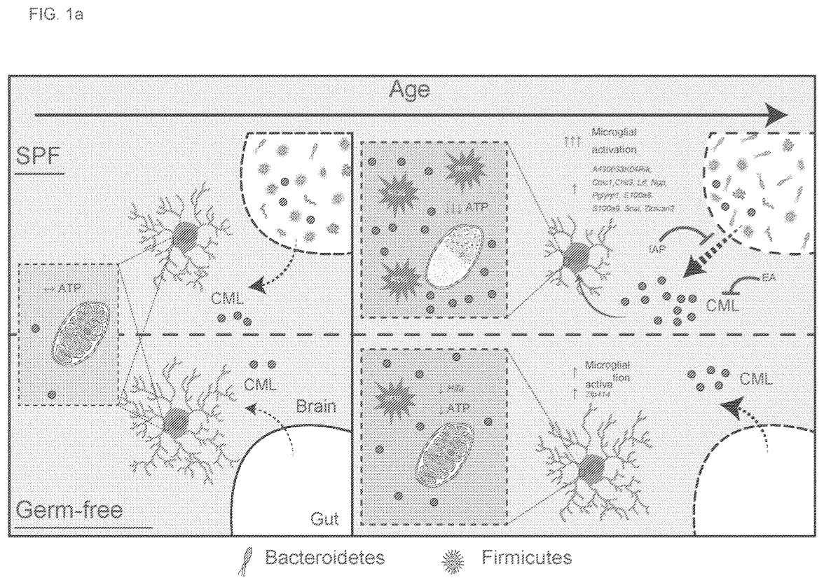

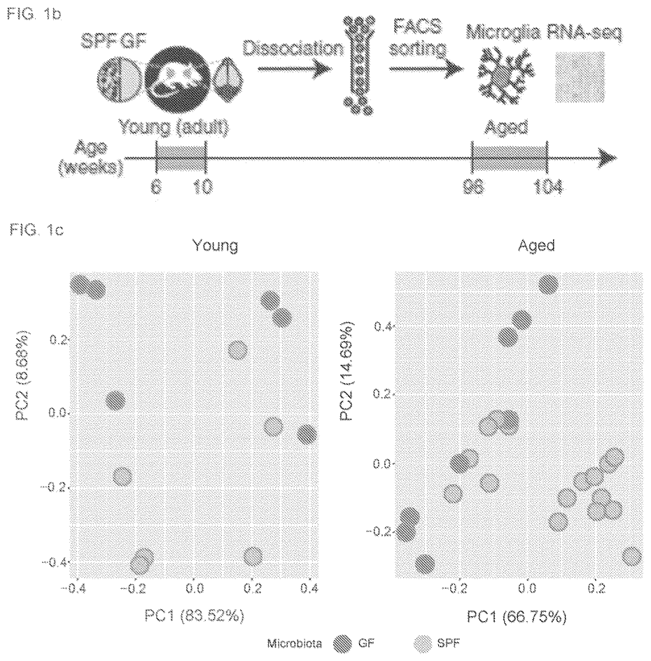

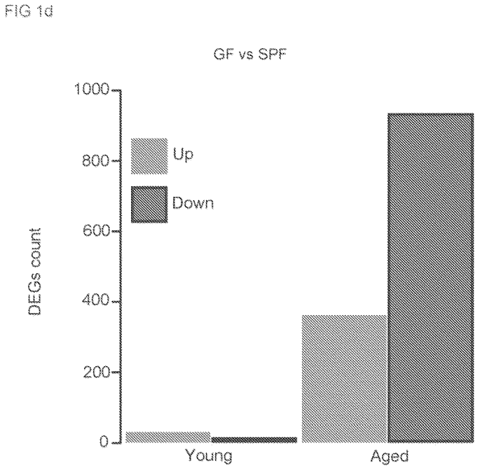

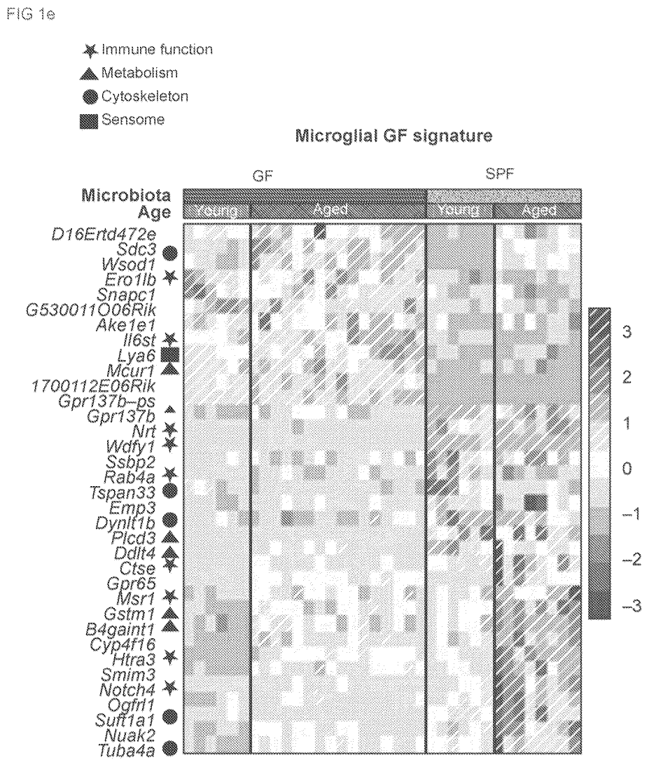

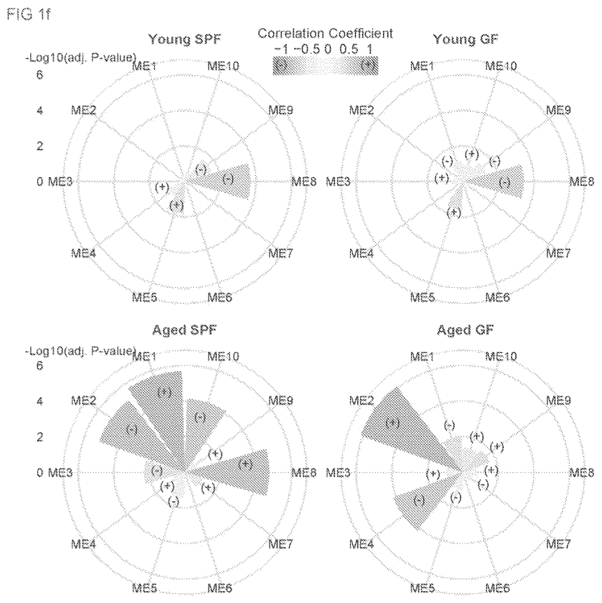

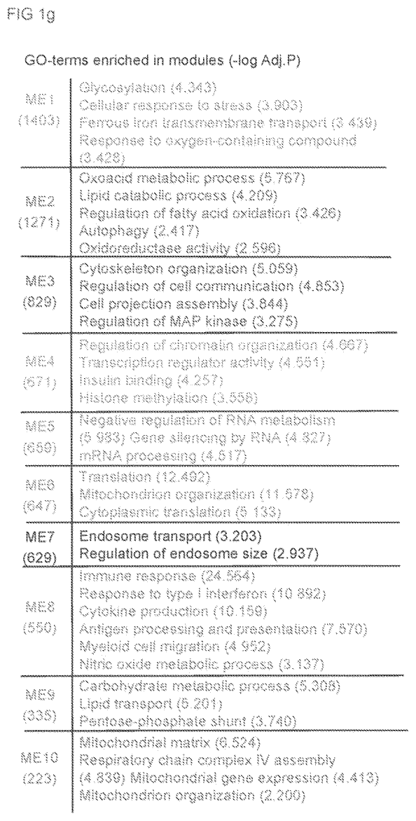

More specifically, as shown schematically in FIG. 1a, it was discovered that mice grown under specific pathogen free (SPF) conditions exhibited greater levels of gut derived CML in their brains relative to mice grown under germ-free (GF) conditions. This resulted in more reactive oxygen species and lower ATP in the brains of the SPF mice versus GF mice. As aging progresses, this results in greater microglial activation in SPF mice versus GF mice. It was also discovered that agents that enhance gut barrier function can slow the release of gut derived CML during the aging process. It was also discovered that agents that eliminate gut microbiota dysbiosis during aging can reduce the amount of gut derived CML produced during the aging process. These agents can reduce the development of cognitive impairment and neurodegenerative disorders.

### Gut Microbiota Alters the Microglial Transcriptomic Profile Upon Aging

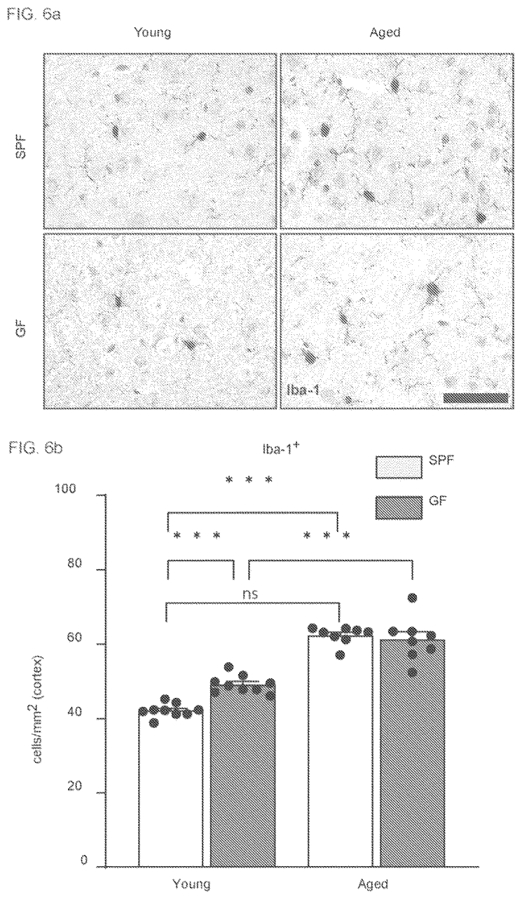

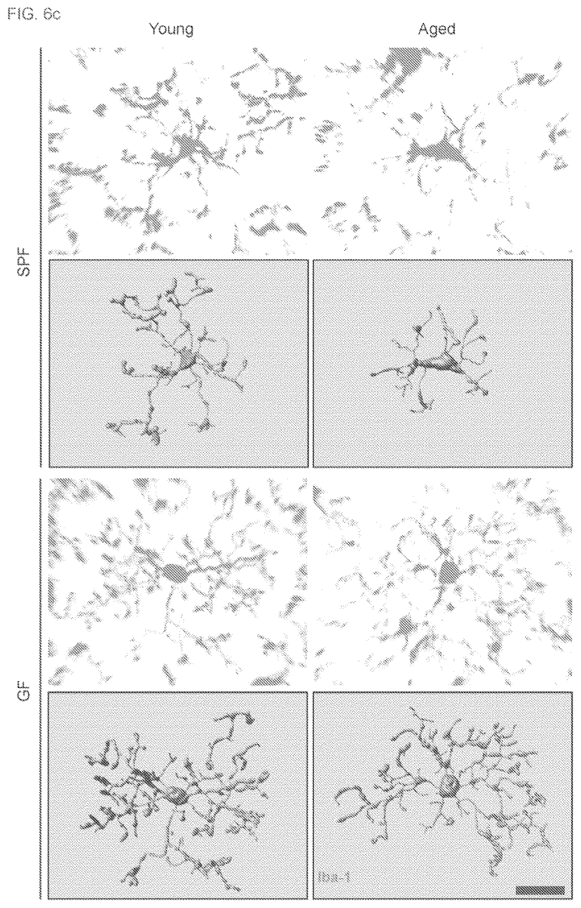

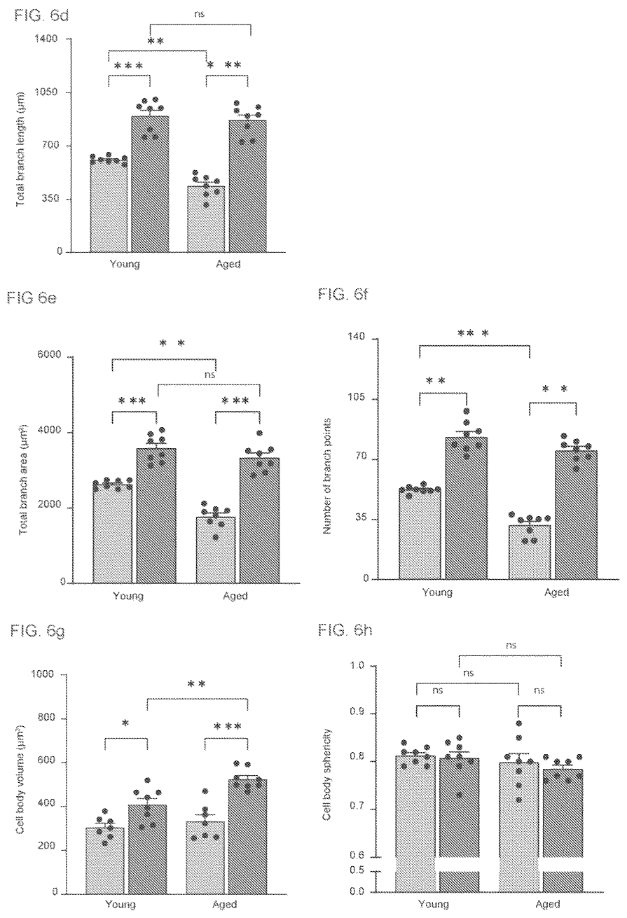

A higher microglial cell density was reported with aging in the brain cortex (Tremblay et al. (2012) GLIA, 60: 541-558). The increased microglial cell density in the cortex of specific pathogen-free (SPF) and germ-free (GF) animals between young-adult and aged mice was found (see, FIGS. 6a-6b) but no difference between aged SPF and GF mice was noted (see, FIGS. 6a-6b). One of the most prominent and first identified features of aging microglia is their change in morphology. To determine the potential morphological changes in microglia of SPF and GF mice, quantitative morphometric reconstruction was performed. The microglia of SPF mice displayed a reduction in total branch area, total branch length and the number of branch points, together with an increase in cell body volume (see, FIGS. 6c-6g) but cell body sphericity was not altered between the groups (see, FIG. 6h). These data demonstrate that age has an effect on microglial morphology in SPF mice, while microglia were unaltered and hyper-ramified under GF conditions.

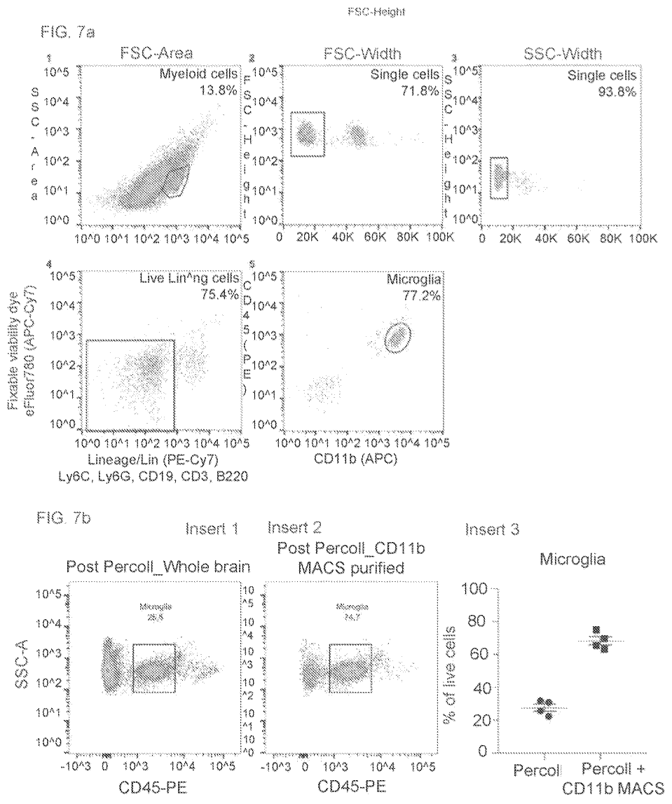

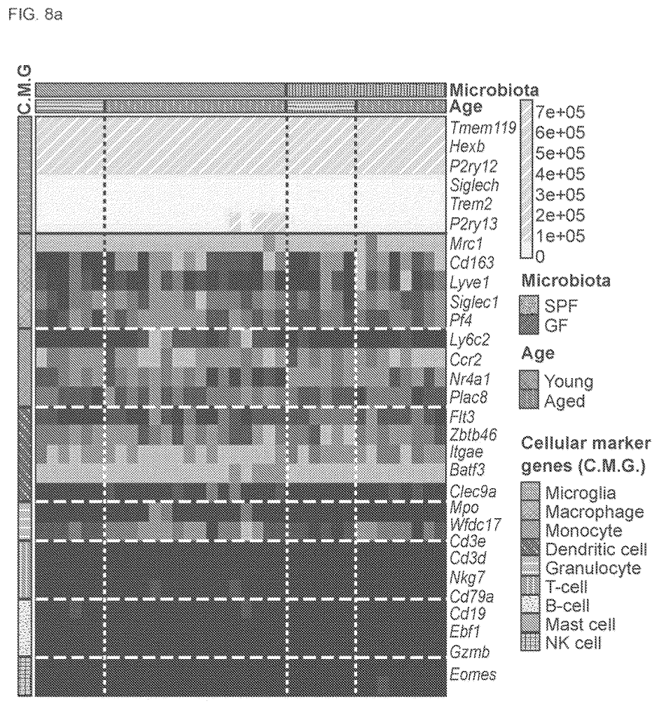

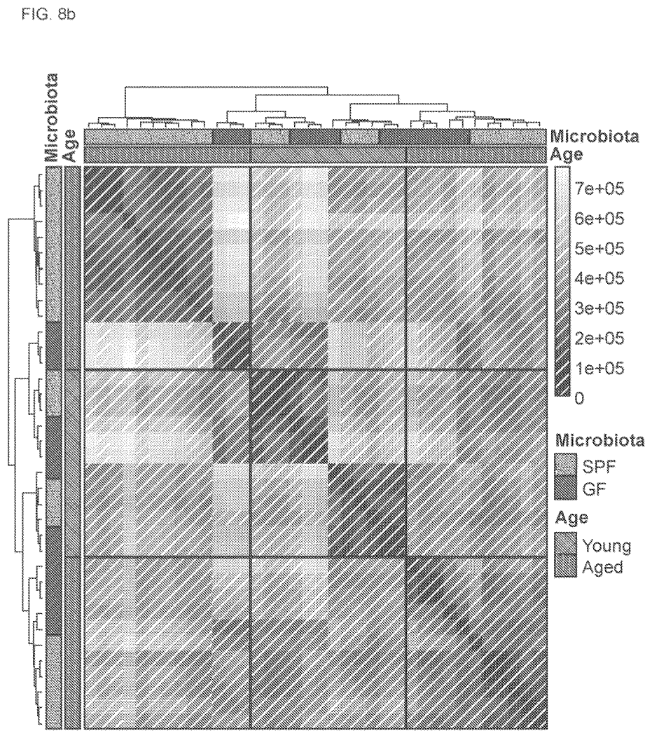

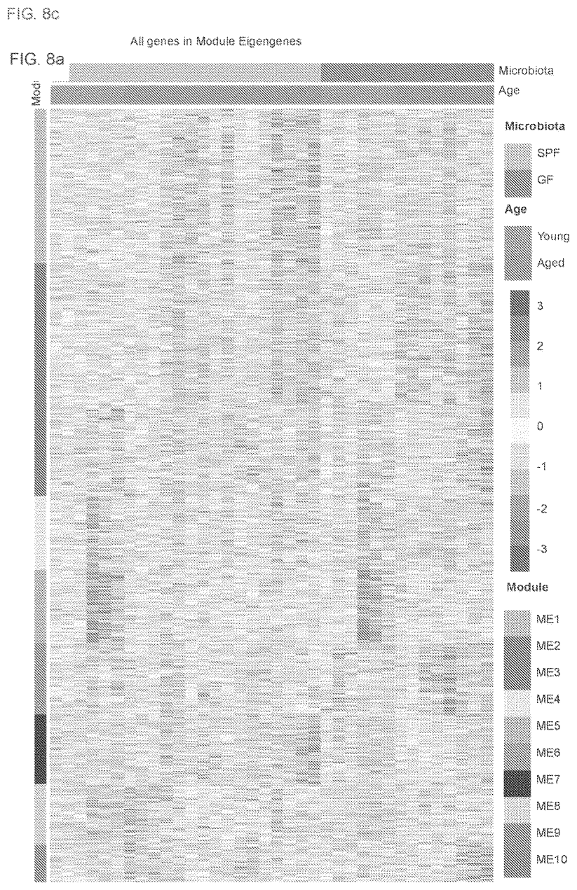

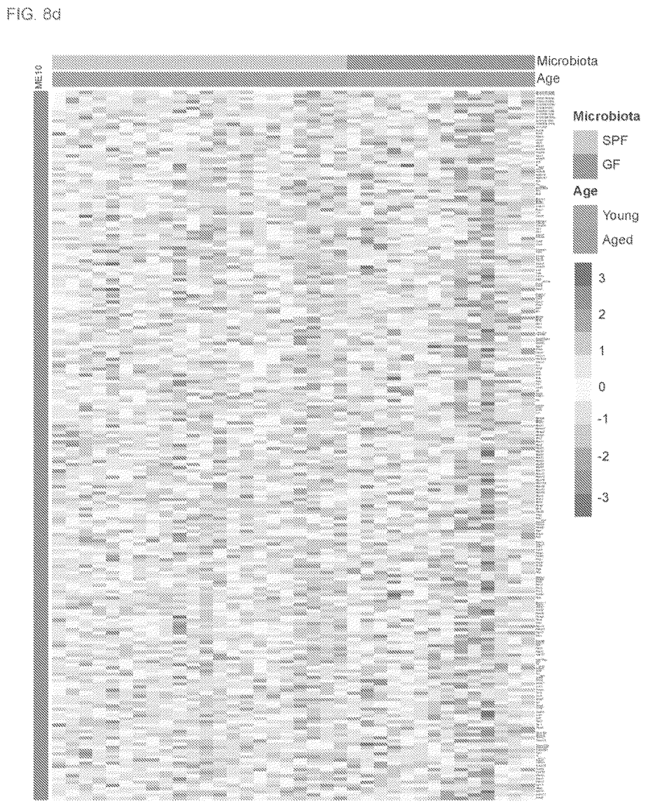

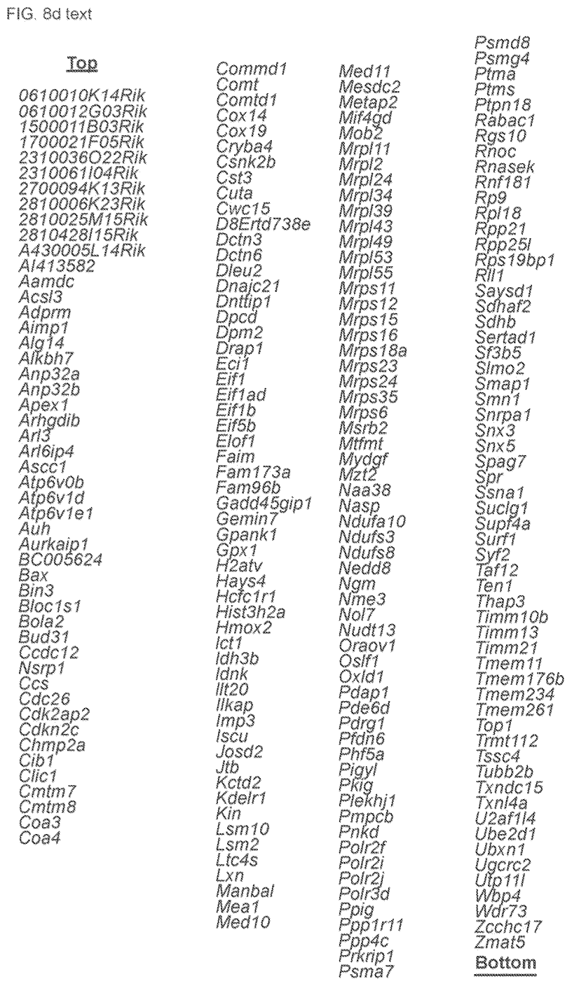

To further evaluate the microbiota-dependent alterations of microglial physiology in the aging brain, RNA-seq was performed on FACS-purified microglia (see, FIGS. 7a and 8a) from the whole brains of young-adult (6-10-week-old) and aged (96-104-week-old) male and female mice, housed under GF or SPF conditions (see, FIG. 1b and FIG. 8b). Distinct gene expression profiles of microglia in GF and SPF mice across both age groups, and transcriptomic differences between microglia isolated from GF and SPF mice were more prominent at older age (see, FIG. 1d). When compared to microglia from SPF mice, an age-independent gene expression pattern emerged in the microglia of GF mice (microglial GF signature), which included genes related to the cytoskeleton (for example, Sdc3, Sult1a1 and Tuba4a) and immune function (for example, Cse, Ero1lb, Htra3, Kcnma1, Notch4, Nr1d2, Rab4a and Wdfv1) (see, FIG. 1e). Moreover, the microglial GF signature contained genes associated with the regulation of mitochondrial function (for example, B4galnt1, Gpr137b, Gstn1, Mcur1, Mtfp1, Nnt and Plcd3), which demonstrates a capacity of the microbiota to regulate the metabolic profile of microglia (see, FIG. 1e). Next, the functional changes in gene expression in microglia with regard to age and microbiota using a weighted gene coexpression network analysis (WGCNA) were characterized (Langfelder et al. (2008) BMC BIOINFORMATICS, 9: 559). Genes that significantly (Wald P_{adj}<0.05) explained more than 50% of the variance were binned into module eigengenes (MEs) based on their coexpression pattern. Upon comparing the young-adult groups under SPF or GF housing (Baker et al. (2011) NATURE, 479: 232-236), minor differences in the gene networks associated with immune function and epigenetic regulation-ME1, ME5, ME6 and ME7, respectively, were observed. However, two modules were characteristic of each age group. ME1 and ME4 in the aged SPF group included genes related to processes like mitochondrial metabolism and lipid localization, while ME2 and ME6 in the aged GF group included genes that regulate the immune response, histone lysine methylation and cell morphogenesis.

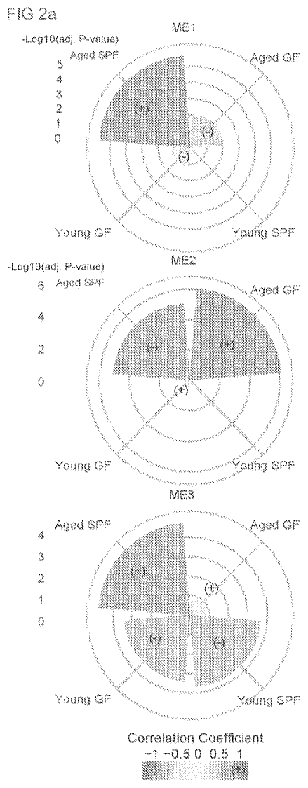

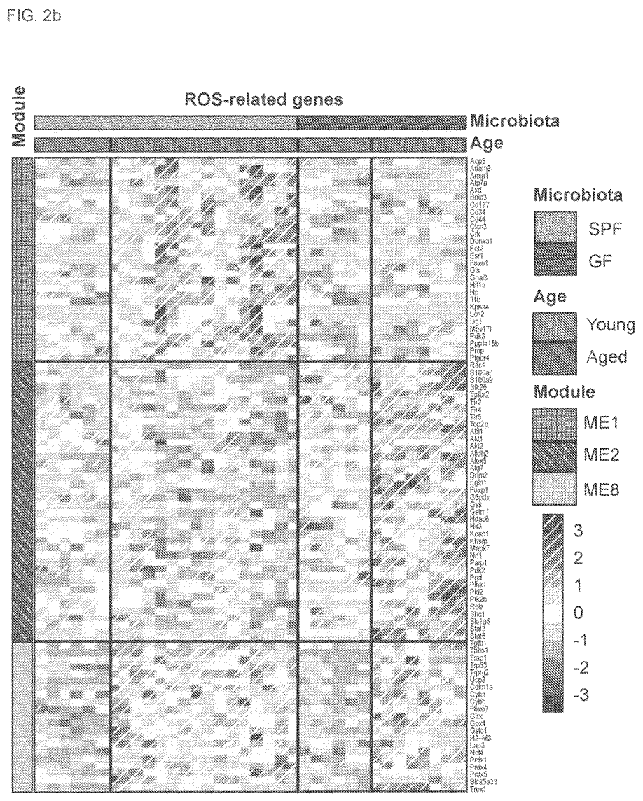

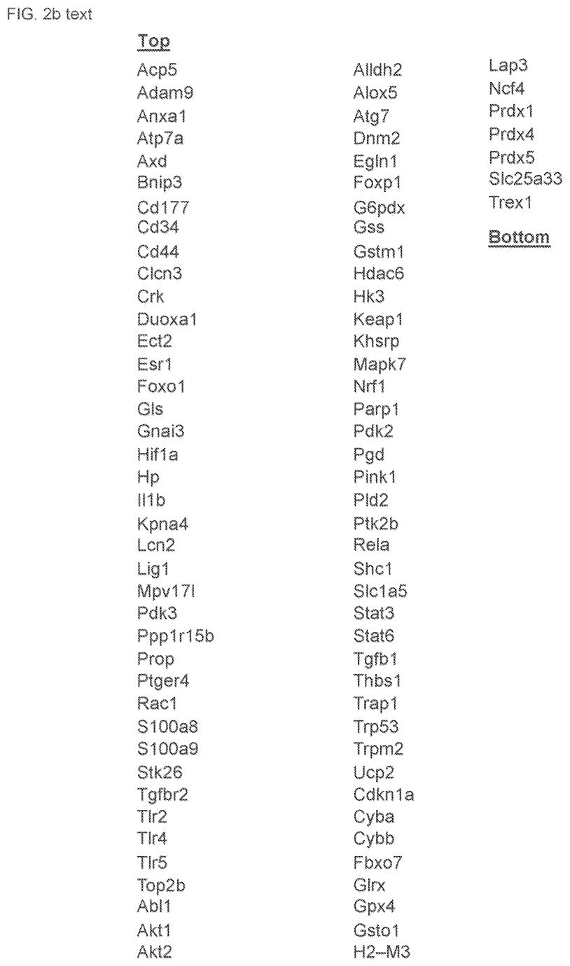

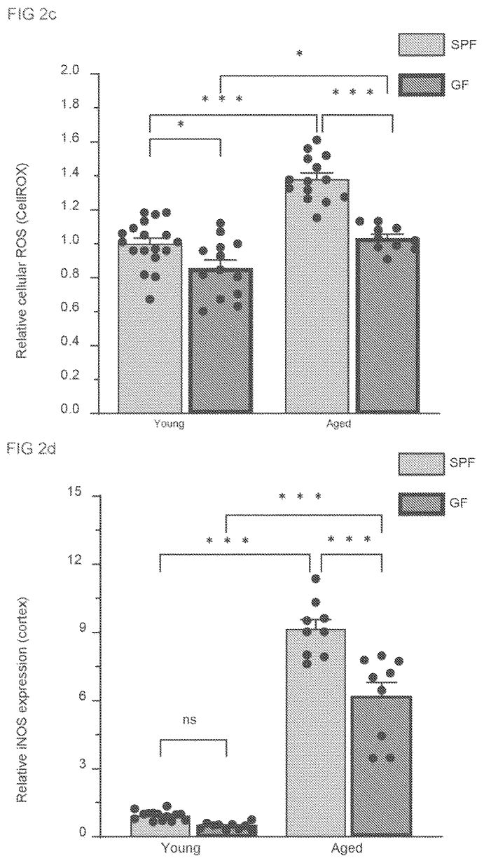

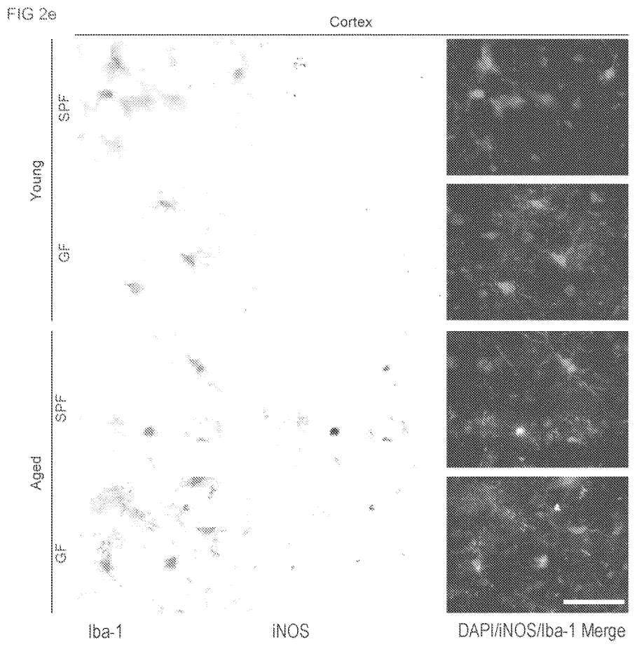

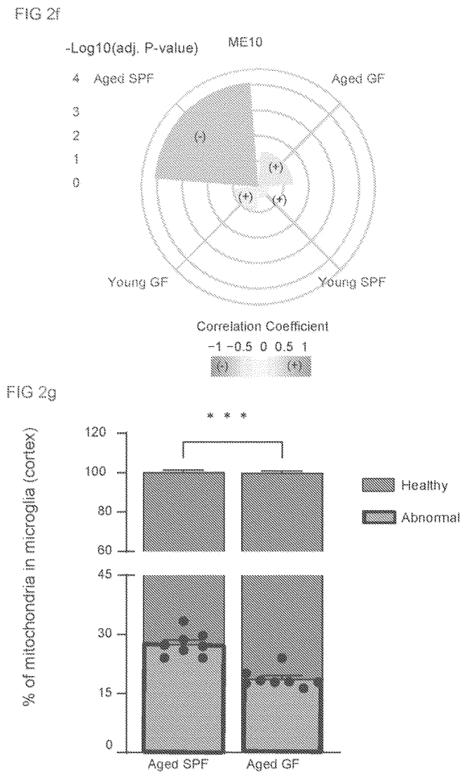

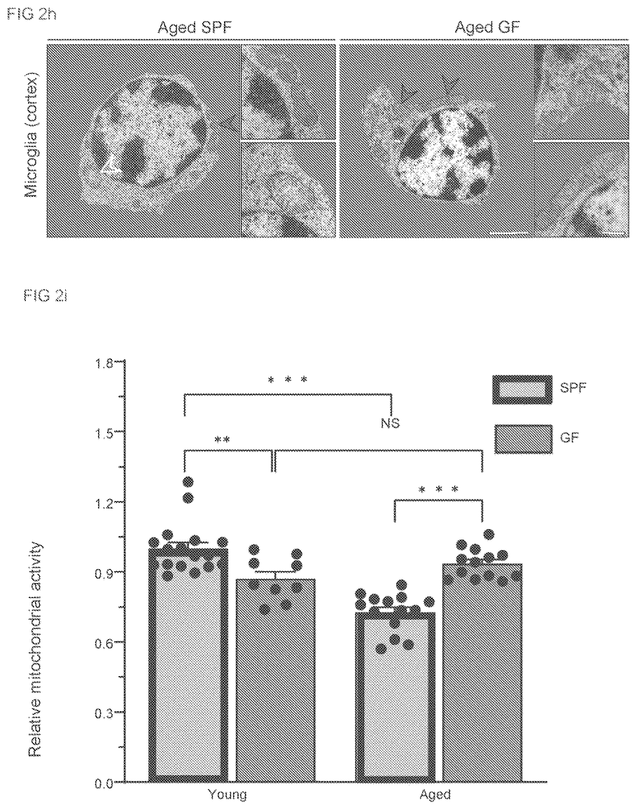

The microbiota's contribution to the age-related MEs was next examined. weighted gene co-expression network analysis (WGCNA) revealed that microglia from aged GF mice followed the general aging trend in SPF mice, but with lower magnitude (see, FIGS. 1f-1g), and clustered closer to the young-adult groups (see, FIG. 8b). For example, genes in ME1 and ME8 associated with immune response (e.g., Axl, Crlf2, Tnfsf8, Tnpf10, Ccl12, Fgr, Il1b, Il6sm, Spp1 and Tlr2), interferon signaling (e.g., Cxcl10/8, Ifi207, Ifit2/8 and Stat1), inflammatory response (e.g., Cd180, Ldlr, S100a8 and S100a9) and microglial cell migration (e.g., Ccl12 and Cxcl10) showed a specific upregulation in the microglia of aged SPF mice, while having negative or low correlation in both young-adult groups and aged GF mice (see, FIG. 2a and FIGS. 8c-8d). ME1 and ME4 in the aged SPF group included genes related to processes like mitochondrial metabolism and lipid localization, while ME2 and ME6 in the aged GF group included genes that regulate regulation of immune response, histone lysine methylation and cell morphogenesis (FIG. 8c). Genes in ME1 & ME8, associated with immune response (e.g., Axl, Crfl2, Tnfsf8, Tnfsf10, Ccl12, Fgr, Il1b, Il6st, Spp1, and 7r2), interferon signaling (e.g., Cxcl10 8, Ifi207, Ifit208, and Stall), inflammatory response (e.g., Cd180, Ldlr, S100a8, and S100a9), and microglial cell migration (e.g., Ccl12 and Cxcl10) showed a specific upregulation in microglia of aged SPF mice, while having negative or low correlation in both young adult groups, and aged GF mice. ME1 and ME8, showed a strong correlation in the aged SPF group, including mitochondrial metabolic processes, hydrogen peroxide metabolic processes and reactive oxygen species metabolic processes. ME2, highly enriched in aged GF mice, was associated with response to oxygen-containing compounds (FIG. 8c). Genes in ME2, which include genes that regulate cellular ROS levels, such as Foxp1, Nrf1, and Trp53 and genes that are implicated in the regulation of mitochondrial ROS, such as G6pdx, Pdk2, Sta13 and Ucp2, were less expressed in the microglia of aged SPF mice when compared to age-matched GF mice, indicating that ROS-levels in aged SPF mice cannot be kept in an optimal cellular range. The accumulation of ROS in the aging brain is associated with mitochondrial damage and mitochondrial dysfunction (Stefanatos et al. (2018) FEBS LETT., 592: 743-758). In ME3, ME5 & ME9, prominent changes were found in genes related to mitochondrial assembly, carbohydrate metabolism, and oxidative phosphorylation. Although these genes were upregulated in SPF and GF mice, mitochondrial damage is more pronounced in SPF mice because here the protective ROS-regulating genes were downregulated. In addition, old GF mice upregulate genes, which are responsible for maintaining mitochondrial structure and function (FIG. 8d). Microglia showed alterations in their transcriptomic profile with aging, with microbiota-dependent divergences.

### Reduced Oxidative Stress in the Microglia of Aged GF Mice

A key feature of cellular aging in microglia is increased oxidative stress, which refers to elevated intracellular levels of reactive oxygen species (ROS) (Streit (2006) TRENDS NEUROSCI., 29: 506-510). Upon inspecting the pathways in the age-related modules, several connections to the regulation of oxidative stress in microglia that were dependent on the microbiota were found. ME1 and ME8 showed a strong correlation in the aged SPF group, including mitochondrial metabolic processes, hydrogen peroxide metabolic processes and ROS metabolic processes. ME2, which was highly enriched in aged GF mice, was associated with response to oxygen-containing compounds (see, FIGS. 1f-1g and FIG. 2a). To confirm that the expression levels of microglial ROS-related genes were regulated by the age of the mice and housing condition, ROS-related genes in the age-related ME1, ME2 and ME8 were selectively analyzed. A specific upregulation of several immune activation and ROS-promoting genes, such as Cdkn1a, Cyba, Cybb, Duoxa1, Il1b, Tgfbr2, Tlr2, Tlr4 and Tlr5, and ROS response genes, such as Axl, Hif1a, Lcn2, Mmp2, Rela, Trex1, S100a8 and S100a9, only in the microglia of aged SPF mice were found (see, FIG. 2b). Genes in ME2, which included genes that regulate cellular ROS levels, such as Foxp1, Nrf1 and Trp53, and genes implicated in the regulation of mitochondrial ROS, such as G6pdx, Pdk2, Sat3 and Ucp2, were less expressed in the microglia of aged SPF mice compared to age-matched GF mice (see, FIG. 2b). ROS production in the microglia isolated from young-adult and aged SPF mice monitored via CellROX flow cytometry assay, and it was found that a significant elevation of ROS were observed with increasing age, which was reduced in aged GF mice (see, FIG. 2c). Activation of inducible nitric oxide synthase (iNOS) appears to be directly linked to the generation of excessive ROS (Sun et al. (2010) ARCH. BIOCHEM. BIOPHYS., 494: 130-137; Zhao et al. (2010) BIOSCI. REP., 30: 233-241). Using immunohistochemistry (IHC), an age-dependent increase in microglial iNOS expression under SPF conditions was observed, which was less pronounced in GF mice (see, FIGS. 2d-2e).

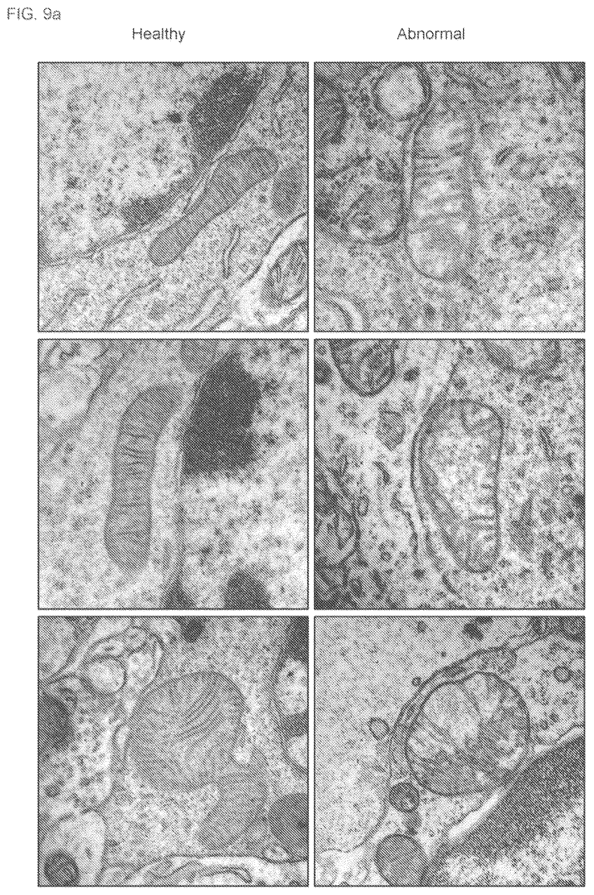

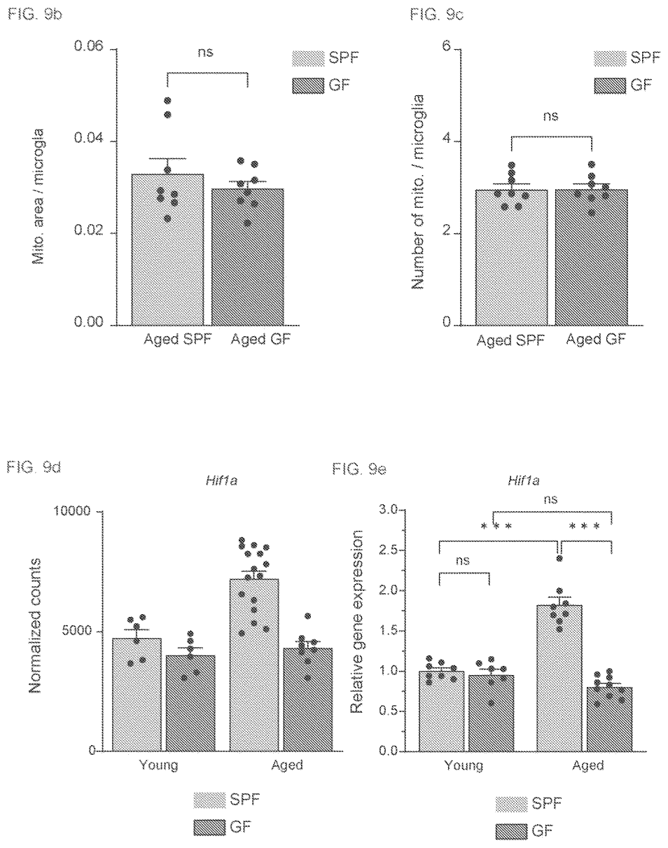

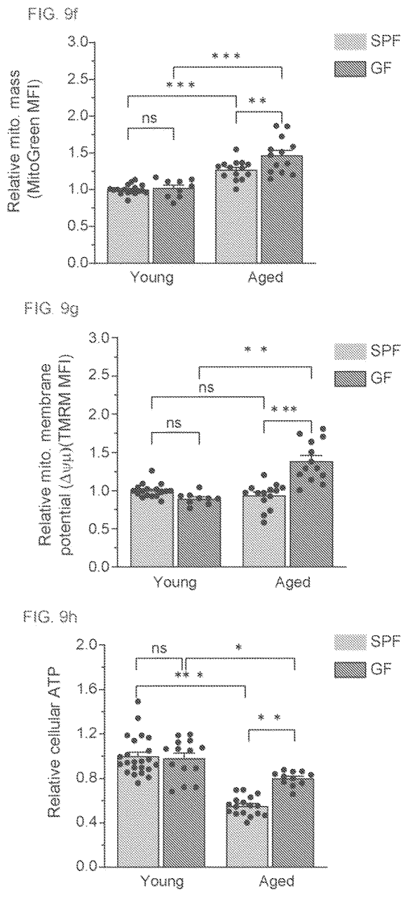

When investigating how the increase in ROS might affect microglial function, it was found that changes in genes related to mitochondrial assembly, carbohydrate metabolism and oxidative phosphorylation occur (see, FIG. 2f). Electron microscopy examination of microglial mitochondria revealed a substantially higher percentage of damaged mitochondria with less well-defined or even destroyed cristae in aged SPF mice relative to aged GF mice, with no change in mitochondrial mass or number per microglia. The accumulation of cellular ROS, which peaked in the microglia of aged SPF mice, appears to induce the expression of hypoxia inducible factor 1 subunit Alpha (Hif1a). Mitochondrial dysfunction in the aged brain leads to a metabolic shift, which is associated with the exaggerated activation of microglia. While young-adult mice showed similar Hif1a expression, RNA-seq and quantitative PCR with reverse transcription (RT-qPCR) indicated higher Hif1a expression in the microglia from aged SPF mice than GF mice (see, FIGS. 9d-9e). The efficiency of oxidative phosphorylation declines in cells of aged animals and leads to reduced ATP production. The transmembrane potential (ΔΨm) of mitochondria appears to be a major driver of ATP production. To account for an increase in mitochondrial mass with aging, mitochondrial activity was plotted as the transmembrane potential of mitochondria relative to the mitochondrial mass. The mitochondrial activity showed an age-associated drop and a reduction in the intracellular ATP reservoir in SPF mice, both of which were less pronounced in the microglia of GF mice (see, FIG. 2i, FIG. 7b, and FIG. 9h). Together, these data demonstrate that the microbiota contributes to increased oxidative stress in the microglia of the aged brain, which is associated with direct damage to the mitochondria.

Microbiota-Dependent Accumulation of CML with Age

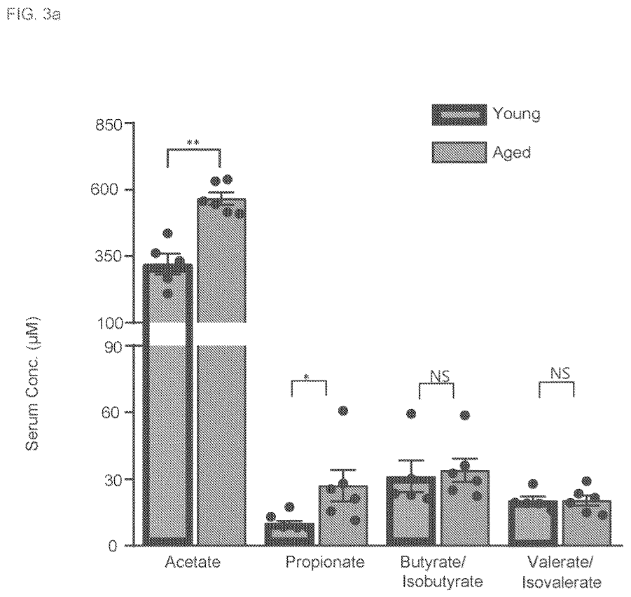

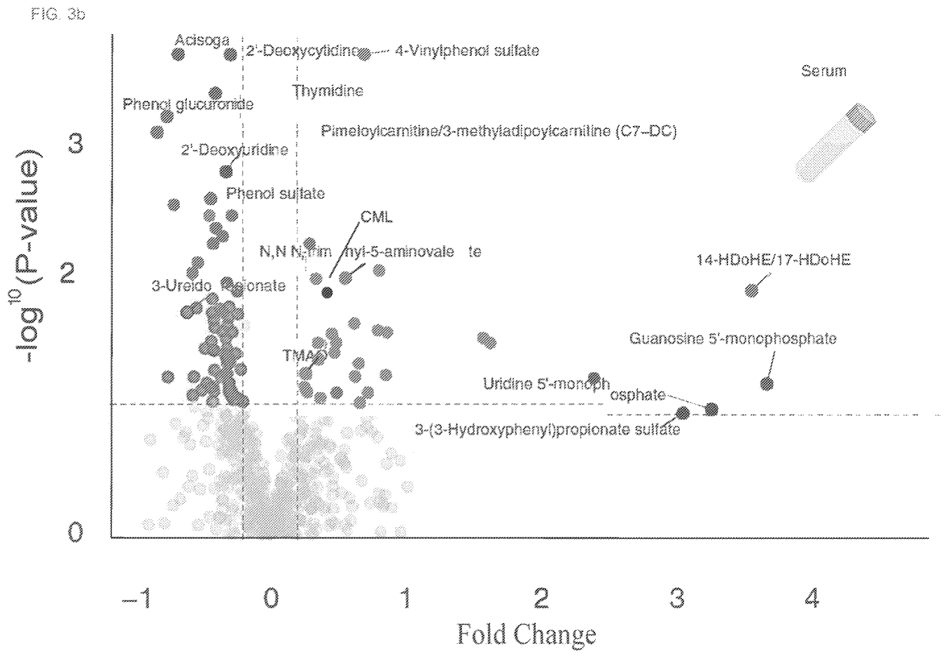

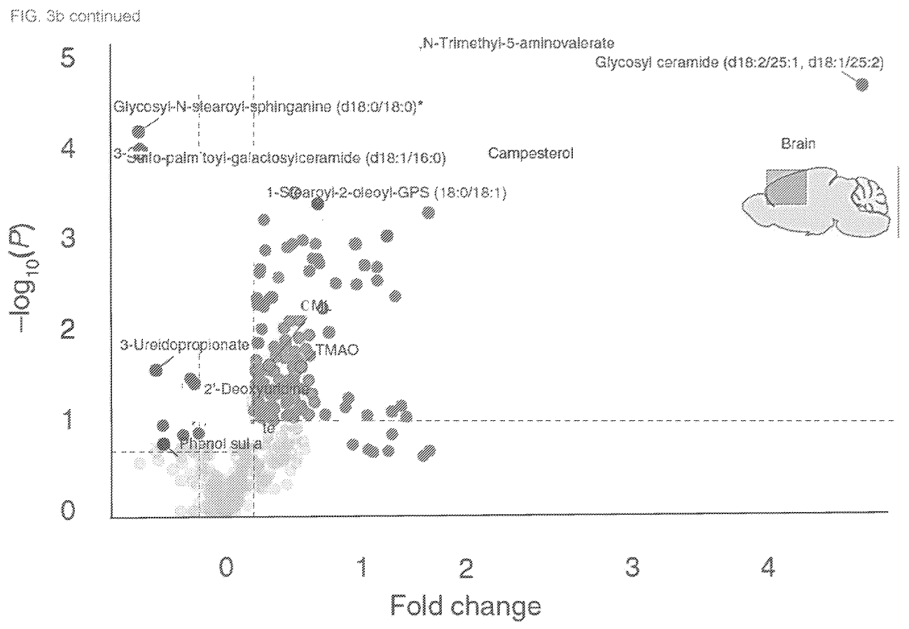

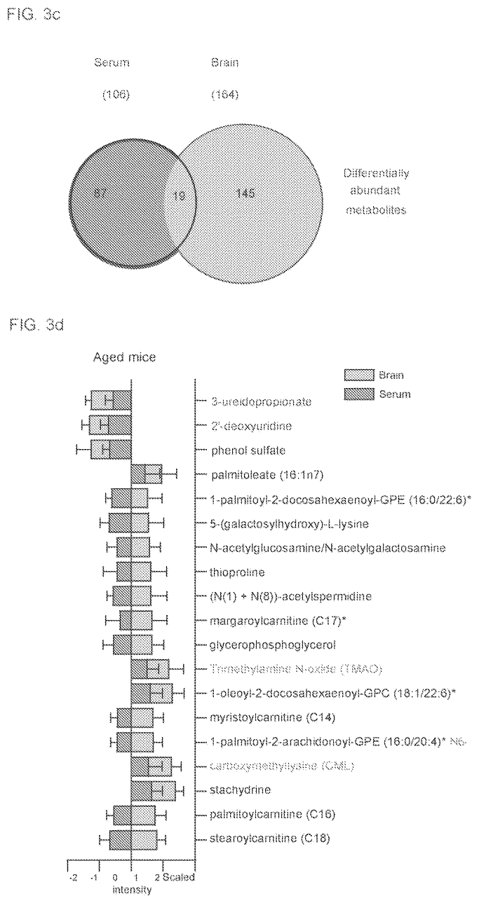

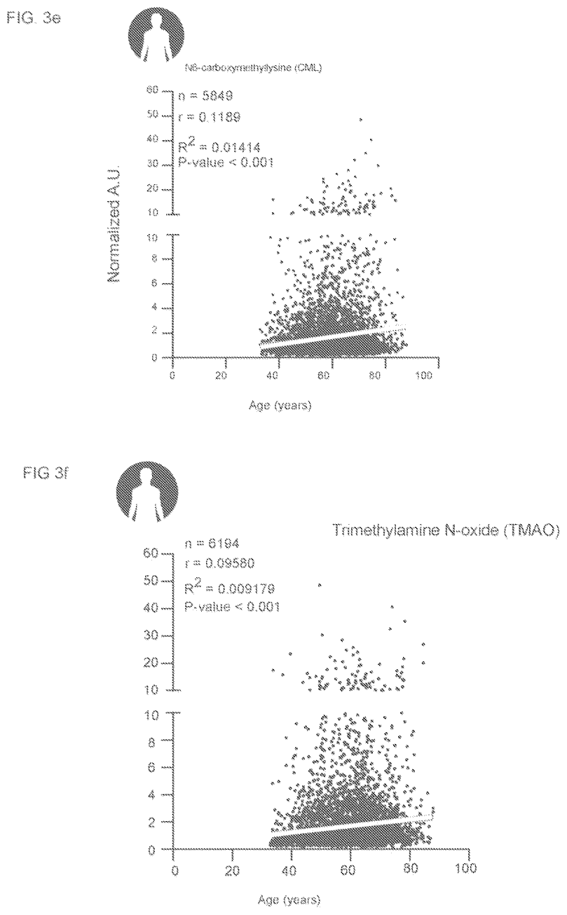

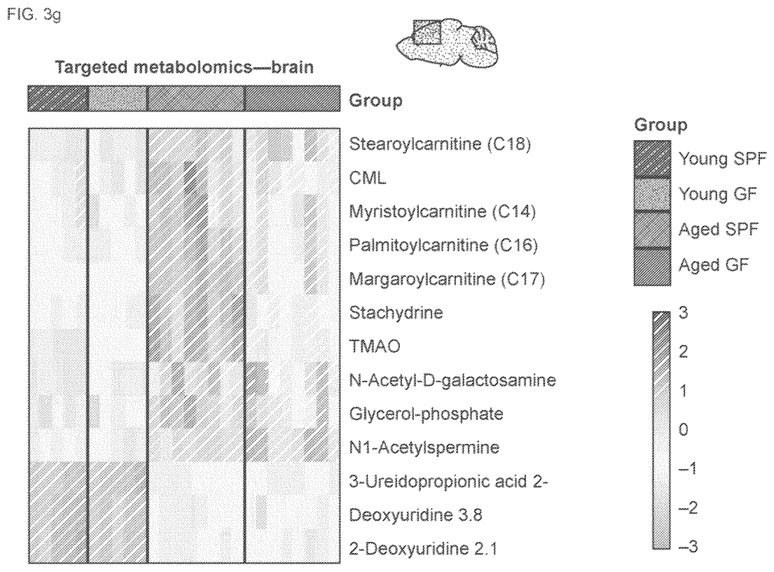

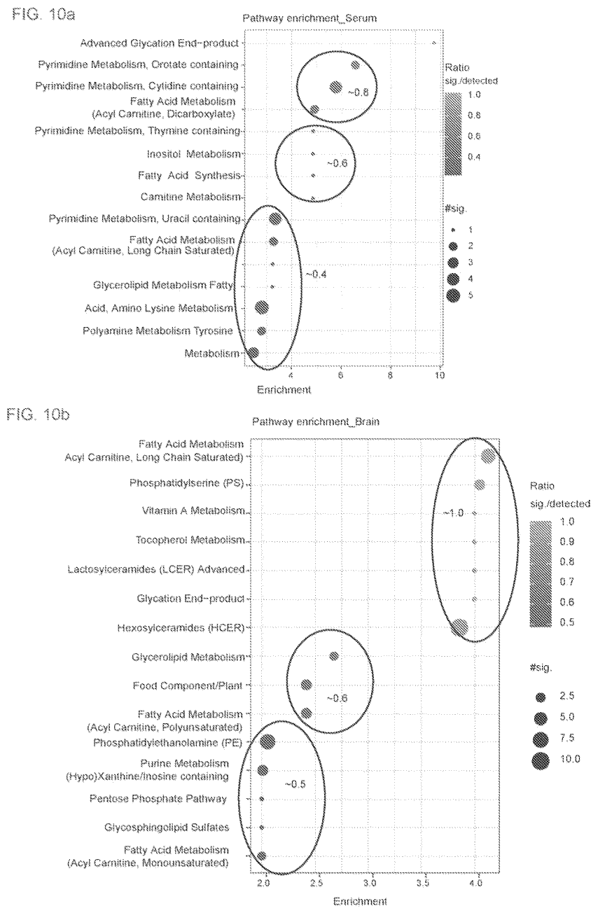

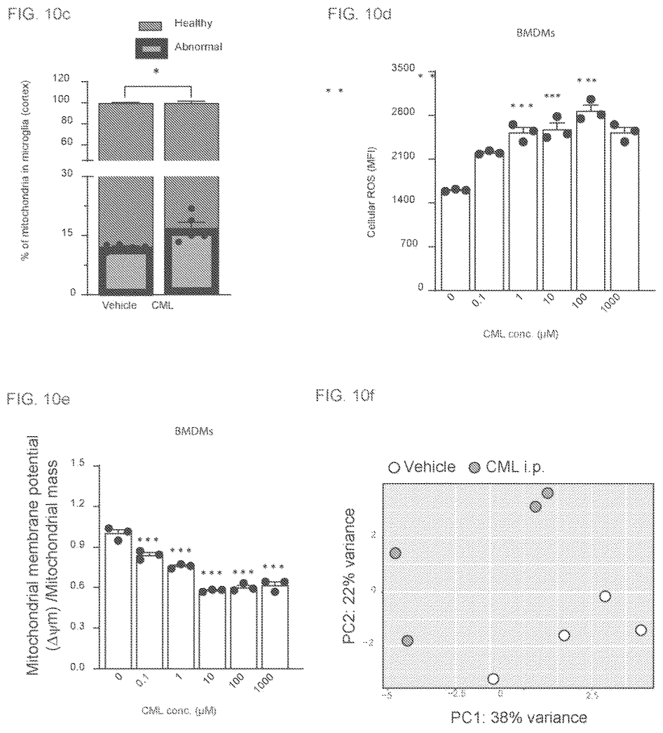

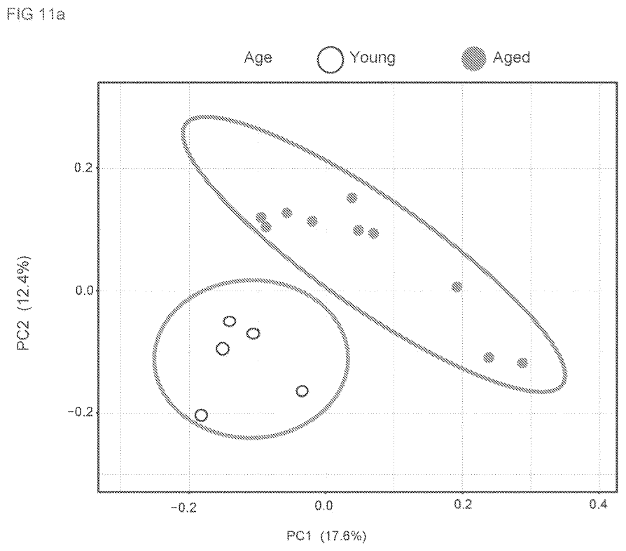

The concentrations of short-chain fatty acids (SCFAs) in the serum samples of young-adult and aged SPF mice were identified using targeted liquid chromatography-mass spectrometry (LC-MS) metabolite analysis (see, FIG. 3a). Acetate was the most abundant SCFA in sera of young adult and aged mice, and elevated concentrations of both acetate and propionate were found in the sera of aged mice compared to young-adult mice. Butyrate/isobutyrate and valerate/isovalerate were not changed. In an unbiased screening, a nontargeted metabolomics dataset was used and blood serum and brain samples from young-adult and aged mice housed under SPF conditions were studied (see, FIGS. 3b-3c) (see, Mossad et al. (2021) NAT. AGING, 1: 1127-1136). Pathway enrichment analysis revealed several tissue-specific pathway alterations. For example, pyrimidine, inositol, carnitine, and several pathways related to amino acid metabolism (e.g., lysine, polyamine and tyrosine) were more affected in the serum of aged mice (see, FIG. 10a). Vitamin A, tocopherol, purine metabolism, ceramide-related pathways, and the pentose phosphate pathway were specifically altered in the brains of aged mice (see, FIG. 10b). Pathways of fatty acid metabolism and advanced glycation end products (AGEs) were commonly altered in both serum and brain samples of aged mice (see, FIGS. 10a-10b). Metabolites that were significantly upregulated in both the serum and brain tissue of aged mice enabled identification of metabolites that were potentially regulated by the gut and reached the brain via the bloodstream. These metabolites included palmitoleate (16:1n7), TMAO, 1-oleoyl-2-docosahexaenoyl-glycerophosphorylcholine (18:1/22:6), CML and stachydrine (see, FIG. 3d). Selected age-related concentration changes seen in mice were confirmed in human blood sample. Nontargeted metabolomics on serum/plasma from a human aging cohort (TwinsUK data bank) recapitulated the age-related concentration changes of CML (see, FIG. 3e) and TMAO (see. FIG. 3f) as seen in mice. Targeted metabolomics from the brain tissue of young and aged SPF and GF mice demonstrated that a functional gut microbiota (e.g., in SPF mice) was necessary for the increase in CML and TMAO in aged mouse brain tissue. Aged GF mice displayed only minor changes compared to young GF mice (see, FIG. 3g).

### CML Enhances Age-Related Microglial Dysfunction

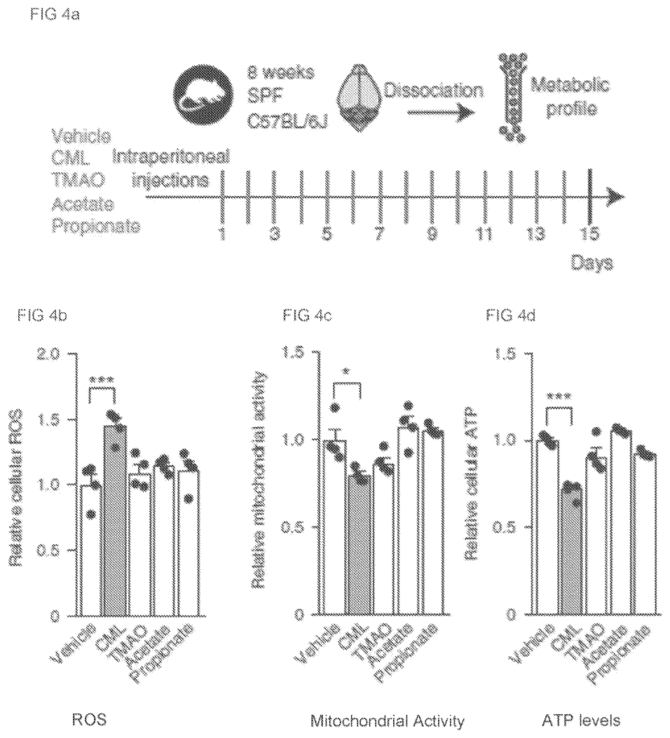

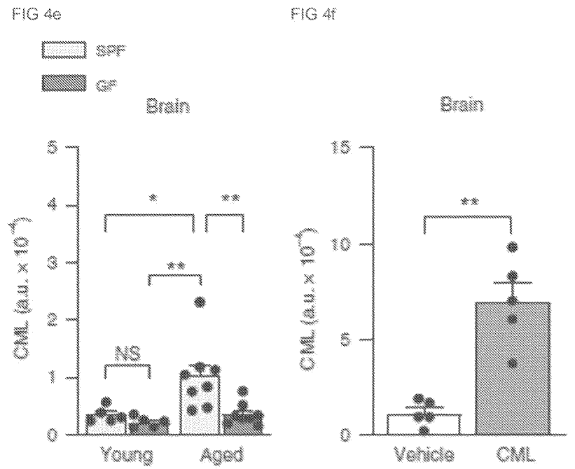

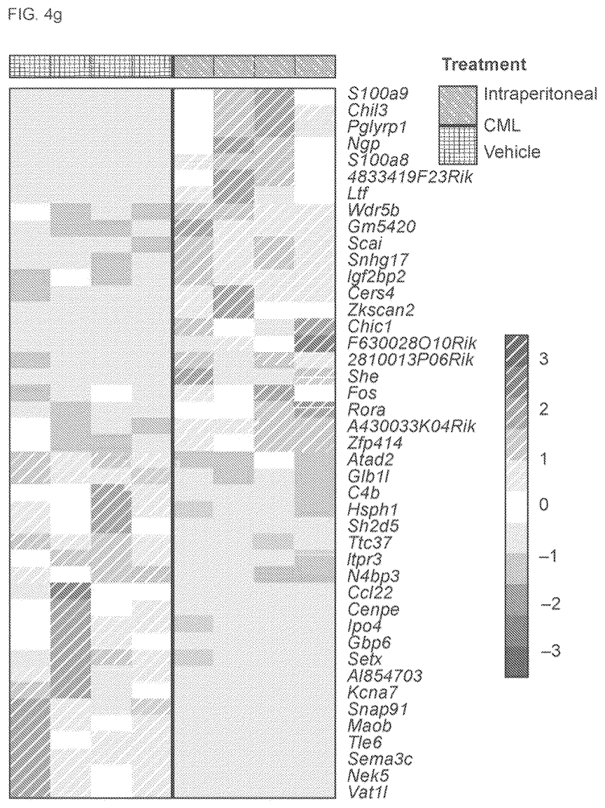

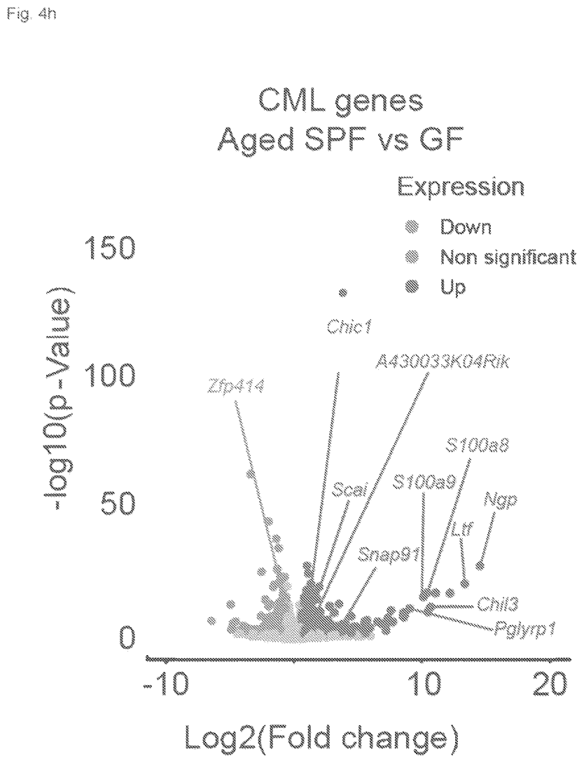

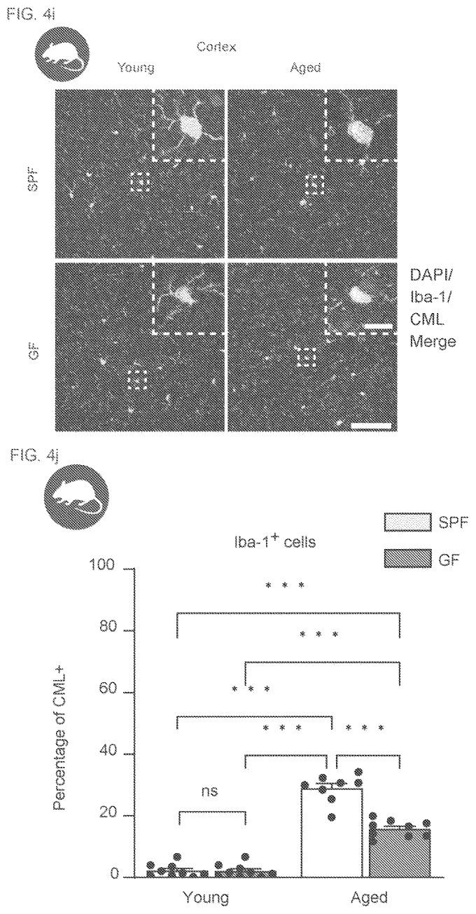

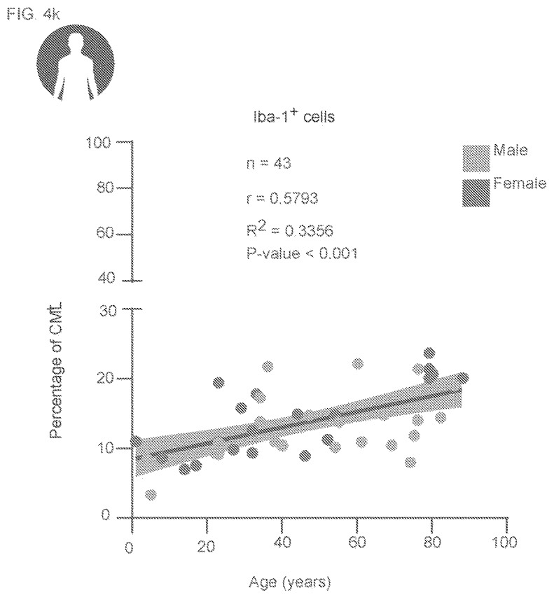

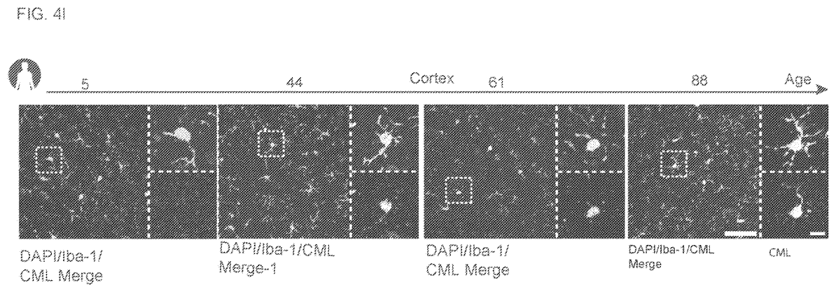

The functional effects of these metabolites on microglia in vivo was next evaluated. CML, TMAO, acetate, and propionate were selected for further evaluation. To identify the metabolite(s) responsible for the increased ROS production in aged microglia, each metabolite was administered separately to young-adult mice. To avoid potential artifacts related to different gut-to-circulation absorption profiles, young-adult mice were injected intraperitoneally once per day for two weeks with CML, TMAO, sodium acetate, or sodium propionate (see. FIG. 4a). TMAO, sodium acetate, and sodium propionate had no effect on intracellular ROS production or on the metabolic function of microglia. However, CML treatment partially recapitulated the changes found in the microglia of aged mice.

CML increased oxidative stress, decreased metabolic activity and reduced cellular ATP stores (see, FIGS. 4b-4d). Moreover, CML caused mitochondrial dysfunction by directly inflicting damage to mitochondrial structures in the microglia (see, FIG. 10c). The impact of CML treatment was not only restricted to microglia but negatively impacted macrophages. In particular, bone marrow-derived macrophages (BMDMs) showed a dose-dependent increase in oxidative stress and decreased metabolic activity in vitro (see, FIGS. 10d-10e). Circulating CML can be derived from an endogenous Maillard reaction, food or the conversion of advanced glycation end products (AGEs) by the gut microbiota. Brain CML levels increased in aged SPF mice but not in aged GF mice (see, FIG. 4e), and CML was still detectable in the brain tissue of young-adult and aged GF mice to a similar level to that of young-adult SPF mice. These results demonstrate that the gut microbiota is required for increased CML levels in the aged brain but not for the baseline levels found in young-adult mice Therefore, these results also demonstrate that the deregulation of mitochondrial function in microglia seen after intraperitoneal injection of CML resulted from increased brain CML concentrations, recapitulating the settings in aged brains (see, FIG. 4). RNA-seq analysis of microglia showed that intraperitoneal injection of CML upregulated the expression of the ROS-related genes S100a9 and S100A8 and other microbiota- and aging-related genes, such as A430033K04Rik, Chic1, Ltf, Ngp, Pglyrp1, Scai and Zkscan2 (see, FIGS. 4g-4h and FIG. 10f).

Immunofluorescence staining of CML in the cortical microglia addressed whether microglia are directly targeted by CML, and showed that mice that were housed under SPF and GF conditions showed an increase in the percentage of CML⁺ microglia with age. Approximately 30% of microglia in aged SPF mice was CML⁺, and the microglia of aged GF mice showed less age-dependent accumulation of CML (see, FIGS. 4i-4j). Furthermore, the age-dependent increase in CML⁺ microglia seen in the mouse cortex was verified to also be present in the human cortex. Human brain tissues (total n=43; males=23, females=20) from individuals between the ages of 1 and 88 years were obtained, and a positive correlation (r=0.5793, R²=0.3356, P<0.001) between age and the percentage of CML⁺ microglia in the human cortex was observed (FIGS. 4k-4l). In mice, RNA-seq analysis of microglia indicated that i.p. injection of CML upregulated the expression of the ROS-related genes, S100a9 and S100A8, and other microbiota- and aging-related genes like A430033k04Rik, Chic1, Ltf, Ngp, Pglyrp1, Scai, and Zkscan2. These findings demonstrate that the age-related accumulation of CML induces microglial metabolic dysfunction in a direct fashion, including increased ROS, and may gradually disrupt brain homeostasis and brain function.

### Aged Microbiota Fuels CML Levels by Disrupting the Gut-Blood Barrier

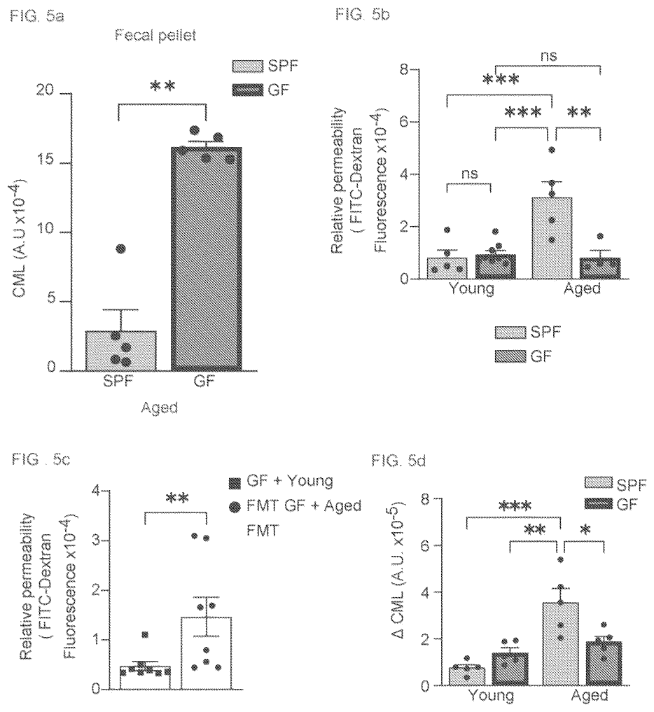

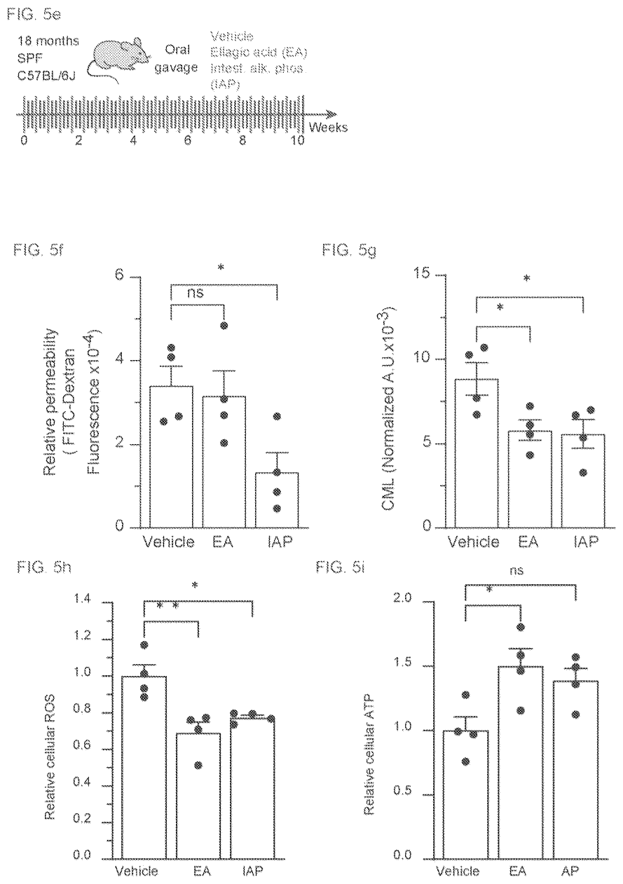

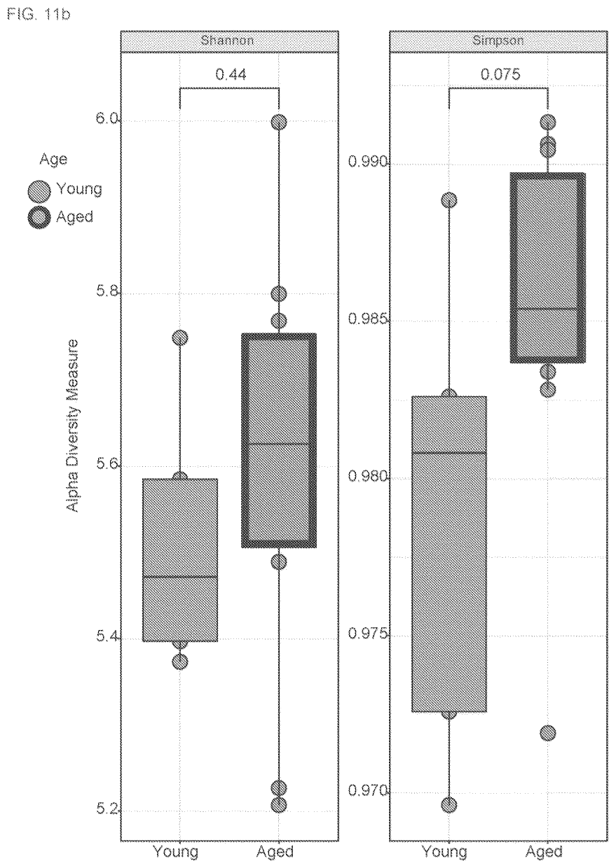

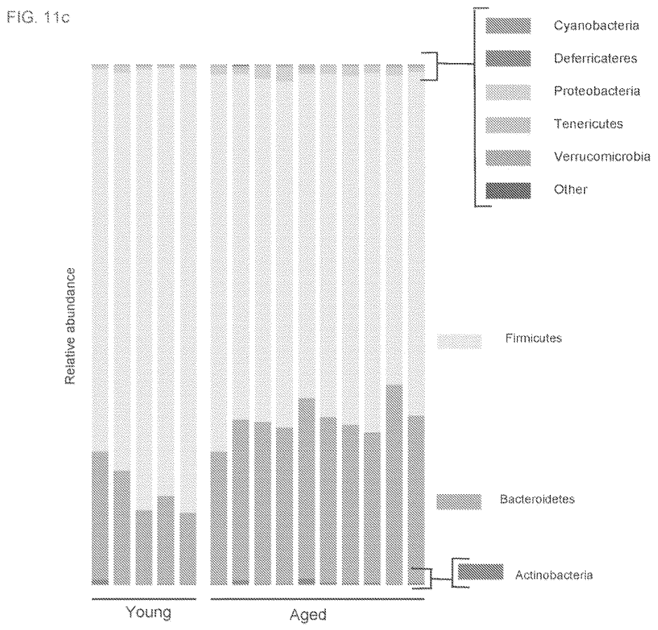

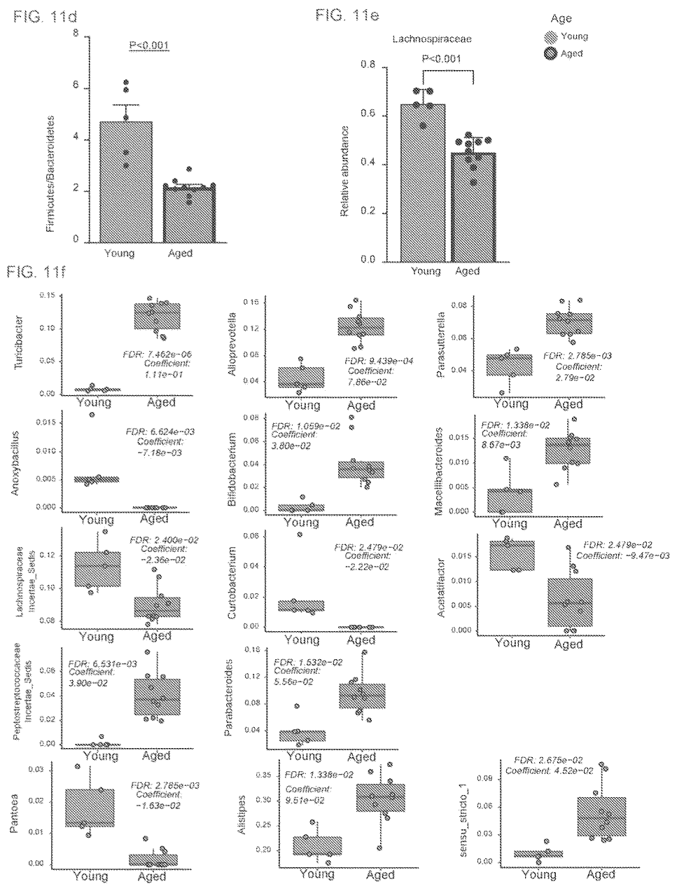

The age-dependent gut microbiota alterations by 16S ribosomal RNA-seq were characterized, based upon the finding that differences in CML levels and microglial function especially at an old age were dependent on the presence or absence of the microbiota. The distinctiveness of the microbiota profile of young-adult and aged mice was confirmed by Beta diversity analysis using the Bray-Curtis dissimilarity metric and the Shannon and Simpson Alpha diversity metrics (see, FIGS. 11a-11b). The gut microbiota in both age groups was dominated by two phyla (see, FIG. 11c), namely Firmicutes and Bacteroidetes. It was noticed that the relative abundance ratio of Firmicutes to Bacteroidetes was altered with advanced age in humans and can be linked to overall changes in bacterial profiles at different age stages. A significant age-dependent reduction in the Firmicutes to Bacteroidetes ratio was observed, where the phylum Firmicutes, family Lachnospiraceae was significantly diminished in aged mice (see, FIGS. 11d-11e). In the bacterial genera, an increased abundance of Turibacter, Alloprevpotella, Parasutterella, Bifidobacterium, Macellibacteroides, Alistipes sensu stricto 1, Peptostreptocccaceae incertaesedis and Parabacteroides was observed in aged mice. This finding was in contrast to the abundance of Pantoea, Anoxybacillus, Lachnospiraceae incertae sedis, Curtobacterium and Acetatifactor, which declined in aged mice (see, FIG. 11f). These findings demonstrate that profiling microbiota in young-adult and aged mice shows alterations at several taxonomic levels. Targeted metabolomics (LC-MS) measurements of CML in fecal pellets revealed that fecal pellets of aged GF mice had higher CML levels than those from aged SPF mice, demonstrating an indirect role of the microbiota in the age-related accumulation of CML in the brain (see, FIG. 5a).

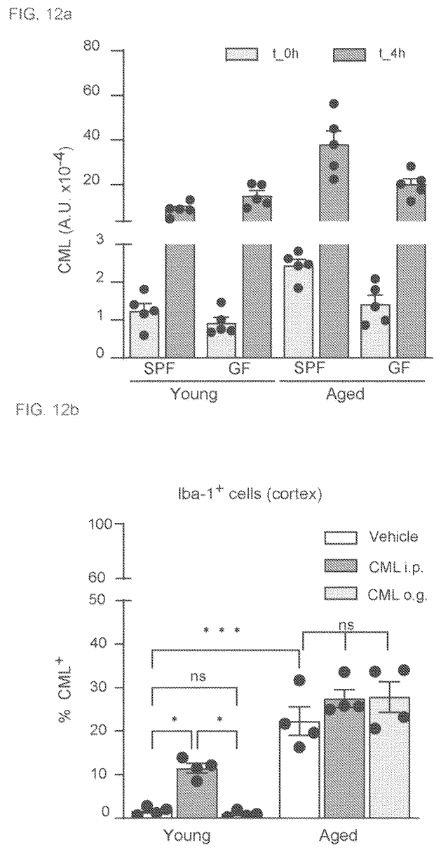

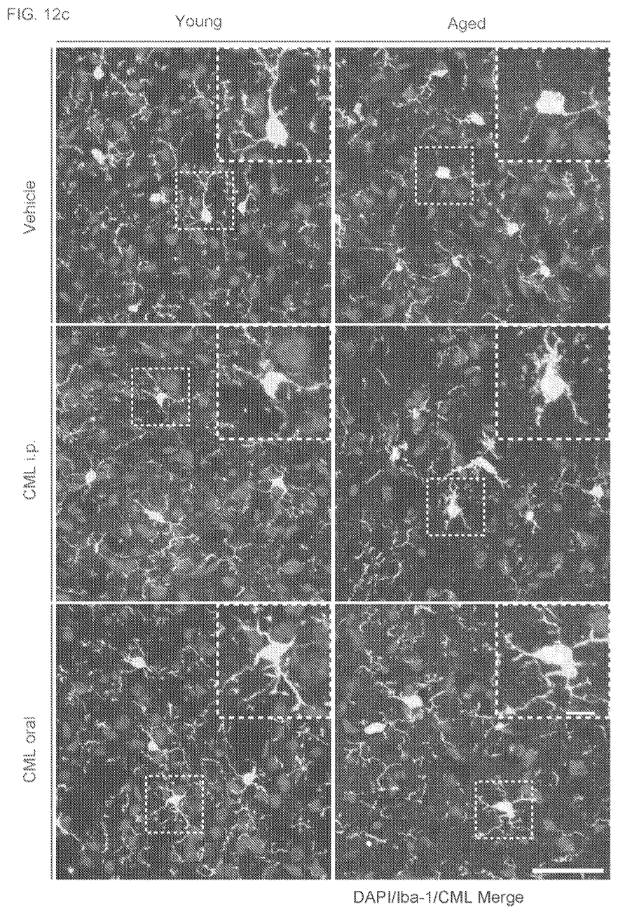

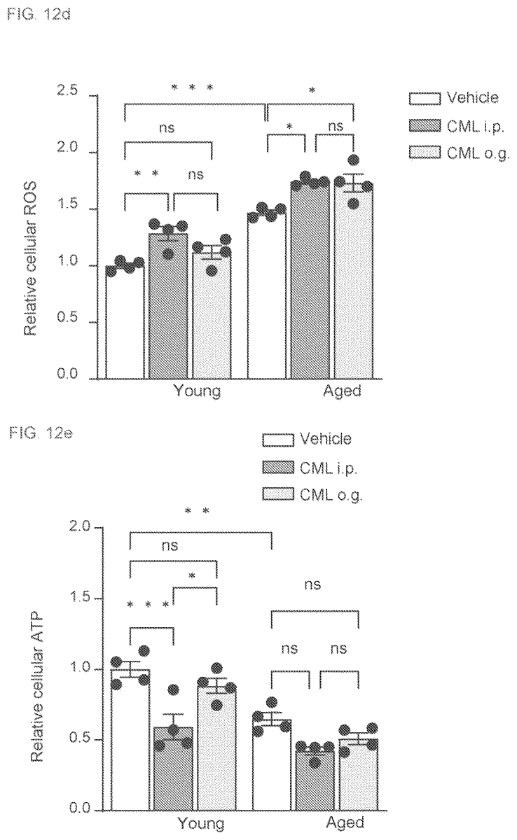

Aged mice show increased intestinal permeability compared to young-adult mice (Hayes et al. (2018) SCI. REP., 8: 14184), a phenomenon that is dependent on the presence of the microbiota (Thevaranjan et al. (2017) CELL HOST MICROBE, 21: 455-466.e4). Increased permeability allows metabolites to pass from inside the gastrointestinal tract through the intestinal epithelium more freely and enter the bloodstream, which may explain the discrepancy between CML levels in the brain and feces. To test this hypothesis, intestinal permeability was measured by quantifying the translocation of FITC-dextran (4 kDa) to the circulation after oral gavage. High gut permeability in aged SPF mice was observed, and the barrier function in aged GF mice was equivalent to that of young-adult SPF and GF mice (see, FIG. 5b). Colonization of young-adult GF mice with aged microbiota induced a microbiota-dependent increase in intestinal permeability compared to the permeability found after young-adult GF mice had received young gut microbiota (see, FIG. 5c). This aligned with the observation that the translocation of CML into the circulation after oral gavage was highest in aged SPF mice (see, FIG. 5d and FIG. 12a). To assess whether different routes of CML application would influence the accumulation of CML in microglia young-adult mice that had received CML intraperitoneally, rather than by oral gavage administration, were studied. Such mice showed more CML⁺ microglia in the cortex (see, FIGS. 12b-12c). In aged mice, the route of CML administration had no effect on the percentage of CML⁺ microglia (see, FIGS. 12b-12c). CML application by both intraperitoneal and oral gavage routes significantly exacerbated the age-related increase in cellular ROS and diminished metabolic function in microglia from aged mice. In young-adult mice, such an effect was only detectable after intraperitoneal administration of CML (see. FIGS. 12d-12e). To verify the key role of the gut barrier to the age-related accumulation of CML in microglia, aged SPF mice (18 months old) were treated every 3 days for 10 weeks orally with ellagic acid (EA), which prevents accumulation of CML (Raghu et al. (2016) FOOD FUNCT., 7: 1574-1583) because it enhances expression of claudins, which are known to cause leakiness of the gut, or intestinal alkaline phosphatase (IAP), an endogenous enhancer of the gut barrier function, by reducing the age-related microbiota dysbiosis and inducing autophagy in the gut epithelium (Kuhn et al. (2020) JCI INSIGHT, 5: e134049: Singh et al. (2020) SCI. REP., 10: 3107)(see, FIG. 5e). While EA had no direct effect on gut permeability, IAP-treated aged mice showed lower gut leakiness (see, FIG. 5f). Both EA and IAP reduced, either indirectly or directly, respectively, CML accumulation in the brain (see, FIG. 5g). The microglia of EA- and IAP-treated aged mice showed a significant reduction in cellular ROS and increased ATP levels compared to vehicle-treated aged mice (see, FIGS. 5h-5i). These findings demonstrate the impact of age-induced microbiota alterations, which disrupt the integrity of the gut barrier and facilitate the accumulation of CML in the brains of aged mice and humans.

Collectively, these observations provide approaches for identifying subjects at risk of developing, or are developing cognitive impairment or a neurogenerative disorder. In addition, these observations provide approaches for treating subjects at risk of developing, or are developing cognitive impairment, or a neurogenerative disorder.

### III. Methods of Treatment

Provided herein are methods of decreasing the rate of development of oxidative stress or mitochondrial dysfunction in microglia, development of mitochondrial dysfunction in microglia, or microglial dysfunction in a subject in need thereof. The methods comprise administering to the subject a therapeutically effective amount of a gut barrier function enhancer and/or an agent for reducing or eliminating gut microbiota dysbiosis, thereby to decrease the rate of development of oxidative stress in microglia, development of mitochondrial dysfunction in microglia, or microglial dysfunction in the subject.

Also provided are methods of treating cognitive impairment or decreasing the rate of development or worsening of cognitive impairment in a subject in need thereof. The methods comprise administering to the subject a therapeutically effective amount of a gut barrier function enhancer and/or an agent for reducing or eliminating gut microbiota dysbiosis, thereby to treat cognitive impairment or decrease the rate of development or worsening of cognitive impairment in the subject.

Also provided are methods of treating a neurodegenerative disease or decreasing the rate of development or progression of a neurodegenerative disease in a subject. The methods comprise administering to the subject a therapeutically effective amount of a gut barrier function enhancer and/or an agent for reducing or eliminating gut microbiota dysbiosis, thereby to treat the neurodegenerative disease or decrease the rate of development or progression of a neurodegenerative disease in the subject.

Also provided are methods of decreasing the rate of development or worsening of neuronal dysfunction in a subject in need thereof. The methods comprise administering to the subject a therapeutically effective amount of a gut barrier function enhancer and/or an agent for reducing or eliminating gut microbiota dysbiosis, thereby to decrease the rate of development or worsening of neuronal dysfunction in the subject.

Also provided are methods of selecting a subject for treatment with a gut barrier function enhancer and/or an agent for reducing or eliminating gut microbiota dysbiosis that include: (a) identifying a subject having an elevated level of CML, a CML precursor, a CML metabolite (also referred to herein as a CML breakdown product), or a CML analog in a biological sample obtained from the subject as compared to a reference level; and (b) selecting the identified subject for treatment with the gut barrier function enhancer and/or the agent for reducing or eliminating gut microbiota dysbiosis. The methods optionally further include determining the permeability of a gut barrier in the subject, and further selecting a subject having an increased level of permeability of the gut barrier as compared to a reference level (e.g., a level of permeability of the gut barrier in a healthy subject) for treatment with the gut barrier function enhancer and/or the agent for reducing or eliminating gut microbiota dysbiosis.

Exemplary CML precursors include (E)-N⁶-((2S,3R,4R,5R)-2,3,4,5,6-pentahydroxyhexylidene)-L-lysine, N⁶-((3S,4R,5R)-3,4,5,6-tetrahydroxy-2-oxohexyl)-L-lysine, L-lysine, and oxalaldehyde.

Exemplary CML metabolites include carboxymethylcadaverine (CM-CAD), 2-amino-6-(formylmethylamino)hexanoic acid, 5-(carboxymethylamino)pentanoic acid, carboxymethyl-cadaverine, carboxymethyl-epicatechin, (5-aminopentyl)glycine, N⁶-(carboxymethyl)-N⁶-(2,3-dihydroxy-5-(3,5,7-trihydroxychroman-2-yl)phenyl)-L-lysine, and N-carboxymethyl-Δ1-piperidinium.

Exemplary CML analogs include Nω-(carboxymethyl)arginine (CMA) and Nε-(1-carboxyethyl) lysine (CEL).

Also provided herein are methods of treating a subject that include administering to the subject a therapeutically effective amount of a gut barrier function enhancer and/or an agent for reducing or eliminating gut microbiota dysbiosis to a subject identified as having an elevated level of CML, a CML precursor, a CML metabolite, or a CML analog in a biological sample obtained from the subject as compared to a reference level. In certain embodiments, the subject has also been identified as having an increased level of permeability of the gut barrier as compared to a reference level (e.g., a level of permeability of the gut barrier in a healthy subject).

Also provided herein are methods of decreasing the rate of accumulation of CML, a CML precursor, a CML metabolite, or a CML analog in a tissue of a subject. The methods comprise administering to the subject a therapeutically effective amount of a gut barrier function enhancer and/or an agent for reducing or eliminating gut microbiota dysbiosis.

Also provided herein are methods of identifying a subject suitable for treatment where the subject has an increased risk of (i) microglial dysfunction, (ii) cognitive impairment, or (iii) developing a neurodegenerative disease. The methods comprise identifying a subject having an elevated level of CML, a CML precursor, a CML metabolite, or a CML analog in a biological sample obtained from the subject as compared to a reference level.

Also provided herein are methods of decreasing the rate of development of oxidative or metabolic stress in microglia in a subject in need thereof. The methods comprise administering to the subject a therapeutically effective amount of a gut barrier function enhancer and/or an agent for reducing or eliminating gut microbiota dysbiosis.

Also provided herein are methods of decreasing the rate of development of mitochondrial dysfunction in microglia in a subject in need thereof. The methods comprise administering to the subject a therapeutically effective amount of a gut barrier function enhancer and/or an agent for reducing or eliminating gut microbiota dysbiosis.

Also provided herein are methods of decreasing the rate of development of microglial dysfunction in a subject in need thereof. The methods comprise administering to the subject a therapeutically effective amount of a gut barrier function enhancer and/or an agent for reducing or eliminating gut microbiota dysbiosis.

Also provided herein are methods of increasing one or more activities or functions of microglia in a subject in need thereof. The methods comprise administering to the subject a therapeutically effective amount of a gut barrier function enhancer and/or an agent for reducing or eliminating gut microbiota dysbiosis.

Also provided herein are methods of decreasing the rate of development or worsening of cognitive impairment in a subject in need thereof. The methods comprise administering to the subject a therapeutically effective amount of a gut barrier function enhancer and/or an agent for reducing or eliminating gut microbiota dysbiosis.

Also provided herein are methods of treating cognitive impairment in a subject in need thereof. The methods comprise administering to the subject a therapeutically effective amount of a gut barrier function enhancer and/or an agent for reducing or eliminating gut microbiota dysbiosis.

Also provided herein are methods of decreasing the rate of development or progression of a neurodegenerative disease in a subject. The methods comprise administering to the subject a therapeutically effective amount of a gut barrier function enhancer and/or an agent for reducing or eliminating gut microbiota dysbiosis.

Also provided herein are methods of treating a neurodegenerative disease in a subject. The methods comprise administering to the subject a therapeutically effective amount of a gut barrier function enhancer and/or an agent for reducing or eliminating gut microbiota dysbiosis.

Also provided herein are methods of decreasing the rate of development or worsening of neuronal dysfunction in a subject in need thereof. The methods comprise administering to the subject a therapeutically effective amount of a gut barrier function enhancer and/or an agent for reducing or eliminating gut microbiota dysbiosis.

Also provided herein are methods of reducing a concentration of CML, a CML precursor, a CML metabolite, or a CML analog in a blood or a brain sample of a subject in need thereof. The methods comprise comprising administering to the subject a therapeutically effective amount of a gut barrier function enhancer and/or an agent for reducing or eliminating gut microbiota dysbiosis.

Also provided herein are methods of reducing a concentration of CML, a CML precursor, a CML metabolite, or a CML analog in a blood or a brain sample to prevent or treat cognitive impairment or a neurodegenerative disease in a subject in need thereof. The methods comprise administering to the subject a therapeutically effective amount of a gut barrier function enhancer and/or an agent for reducing or eliminating gut microbiota dysbiosis, thereby to prevent or treat cognitive impairment or a neurodegenerative disease in the subject.

Also provided herein are methods or reducing a concentration of CML, a CML precursor, a CML metabolite, or a CML analog in a blood or a brain sample to prevent or treat cognitive impairment or a neurodegenerative disease in a subject in need thereof. The methods comprise administering to the subject a therapeutically effective amount of a gut barrier function enhancer and/or an agent for reducing or eliminating gut microbiota dysbiosis, thereby to prevent or treat cognitive impairment or a neurodegenerative disease in the subject.

Also provided herein are methods of reducing permeability of the gut to prevent or treat cognitive impairment or a neurodegenerative disease in a subject in need thereof. The methods comprise administering to the subject a therapeutically effective amount of a gut barrier function enhancer and/or an agent for reducing or eliminating gut microbiota dysbiosis, thereby to prevent or treat cognitive impairment or a neurodegenerative disease in the subject.

In certain embodiments of any of the methods described herein, the subject has previously been identified as having an elevated level of CML, a CML precursor, a CML metabolite, or a CML analog in a biological sample of a subject as compared to a reference level. In certain embodiments of any of the methods described herein, the method further includes identifying the subject as having an elevated level of CML, a CML precursor, a CML metabolite, or a CML analog in a biological sample of the subject as compared to a reference level. In certain embodiments of any of the methods described herein, the biological sample is a tissue of body fluid sample. In some embodiments, the body fluid sample is saliva, urine, blood, serum, plasma, cerebrospinal fluid, or feces. In some embodiments, the tissue sample is brain tissue.

In certain embodiments of any of the methods described herein, the subject has previously been identified as having an increased level of permeability of the gut barrier as compared to a reference level (e.g., a level of permeability of the gut barrier in a healthy subject). In certain embodiments of any of the methods described herein, the method further includes identifying the subject as having an increased level of permeability of the gut barrier as compared to a reference level (e.g., a level of permeability of the gut barrier in a healthy subject). In certain embodiments of any of the methods described herein, the subject has been identified or diagnosed as having cognitive impairment. In certain embodiments of any of the methods described herein, the subject has been identified as having an increased risk of developing cognitive impairment. In certain embodiments of any of the methods described herein, the subject has been identified or diagnosed as having a neurodegenerative disease. In certain embodiments of any of the methods described herein, the subject has been identified as having an increased risk of developing neurodegenerative disease.

Methods for identifying an increased level of permeability of the gut barrier can include analytical techniques known in the art, and include, for example, methods whereby the amount of orally ingested compounds (e.g., labeled or unlabeled compounds) are measured in a tissue or body fluid sample of a subject. In one approach, labeled compounds (e.g.. metabolites labeled with a radioactive or fluorescent label, such as chromium-51 or fluorescein isothiocyanate (FITC), respectively) are orally ingested by a subject. Following administration, the uptake of the labeled compound is measured in a biological sample (e.g., blood or urine) harvested from the subject. The amount of the labeled compound is a measure of the gut permeability of the subject. Alternatively, or in addition, gut permeability can be measured using the lactulose:mannitol (LM) excretion test. The LM excretion test is a quantitative assay for directly measuring the ability of two non-metabolized sugar molecules (lactulose and mannitol) to permeate the intestinal mucosa, thereby measuring the permeability of the gut barrier. In an exemplary LM test, the results can be expressed as the ratio of the amount of ingested lactulose and mannitol to the amount of lactulose and mannitol excreted in the urine. This can be measured using the formula:

Cumulative excretion at time (t)=[excreted concentration of sugar (mg/mL) at t]×total urine volume (mL) at t.

The cumulative excretion of each sugar up to time t (mg) is then expressed as a percentage by dividing the cumulative excretion (mg) by the total amount of sugar ingested (mg)×100.

For example, if the total amount of ingested lactulose is 5 grams and the total amount of ingested mannitol is 1 gram, then normal values for lactulose and mannitol excretion in urine in healthy subjects can be about 0.35% (ranging from 0.020% to 1.803%) for lactulose and 12.3% (ranging from about 1.480% to 43.75%) for mannitol. Values higher than these average values are indicative of a higher gut permeability in a subject.

In certain embodiments of any of the methods described herein, the method results in a reduction in level of cellular and/or mitochondrial reactive oxygen species (ROS) in microglia in the subject. In certain embodiments of any of the methods described herein, the method results in a reduction in expression of inducible nitric oxide synthase (iNOS) in microglia in the subject. In certain embodiments of any of the methods described herein, the method results in a reduction in expression of one or more genes in microglia of the subject selected from the group consisting of Cdkn1a, Cyba, Cybb, Duoxa1, Il1b, Tgfr2, Tlr2, Tlr4, Tlr5, Axl, Hif1a, Lcn2, Mmp2, Rela, Trex1, S100a8, and S100a9. In certain embodiments of any of the methods described herein, the method results in an increase in expression of one or more genes in microglia of the subject selected from the group consisting of Foxp1, Nrf1, Trp53, G6pdx, Pdk2, Stat3, and Ucp2. In certain embodiments of any of the methods described herein the method results in an increase in one or more activities of microglia in the subject.

Some embodiments of any of the methods described herein further include determining the level of CML, a CML precursor, a CML metabolite, or a CML analog in the biological sample obtained from the subject. Some embodiments of any of the methods described herein further include determining a level of permeability of the gut barrier in the subject.

In certain embodiments of any of the methods described herein, the neurodegenerative disease is selected from, but not limited to, the group of Alzheimer's disease, Parkinson's disease, Huntington disease, frontotemporal dementia, amyotrophic lateral sclerosis, multiple sclerosis, glaucoma, myotonic dystrophy, progressive supranuclear palsy, spinal muscular atrophy, multisystem atrophy, ataxias, vascular dementia, or other dementias.

In certain embodiments of any of the methods described herein, following administration of the gut barrier function enhancer and/or an agent for reducing or eliminating gut microbiota dysbiosis, the subject exhibits one or more of: (a) a reduced concentration of CML, a CML precursor, a CML metabolite, or a CML analog in a blood sample; (b) a reduced concentration of CML, a CML precursor, a CML metabolite, or a CML analog in a brain tissue sample; (c) a reduced gut permeability; (d) a reduction of microbiota dysbiosis; (e) an increased level of autophagy in gut epithelium; (f) a reduction in level of cellular and/or mitochondrial ROS in microglia; (g) an increased level of adenosine triphosphate (ATP) in a population of microglia; (h) a reduction in expression of iNOS in microglia; (i) a reduction in expression of one or more genes in microglia selected from the group consisting of Cdkn1a, Cyba, Cybb, Duoxa1, Il1b, Tgfbr2, Tlr2, Tlr4, Tir5, Axl, Hif1a, Lcn2, Mmp2, Rela, Trex1, S100a8, and S100a9; and (j) an increase in expression of one or more genes in microglia selected from the group consisting of Foxp1, Nrf1, Trp53, G6pdx, Pdk2, Stat3, and Ucp2.

Also provided herein are methods of identifying a subject as having an increased risk of developing microglial dysfunction. The method comprises identifying a subject having an elevated level of CML, a CML precursor, a CML metabolite, or a CML analog in a biological sample obtained from the subject as compared to a reference level, wherein such elevated level is indicative the subject has an increased risk of developing microglial dysfunction.

In certain embodiments, the method further includes identifying a subject having an increased level of permeability of the gut barrier as compared to a reference level (e.g., a level of permeability of the gut barrier in a healthy subject) as having an increased risk of developing microglial dysfunction.

Also provided herein are methods of identifying a subject as having an increased risk of cognitive impairment. The methods comprise identifying a subject having an elevated level of CML, a CML precursor, a CML metabolite, or a CML analog in a biological sample obtained from the subject as compared to a reference level, wherein such elevated level is indicative the subject has an increased risk of developing cognitive impairment. In certain embodiments, the method further includes identifying a subject having an increased level of permeability of the gut barrier as compared to a reference level (e.g., a level of permeability of the gut barrier in a healthy subject) as having an increased risk of developing cognitive impairment.

Also provided herein are methods of identifying a subject as having an increased risk of developing a neurodegenerative disease. The methods comprise identifying a subject having an elevated level of CML, a CML precursor, a CML metabolite, or a CML analog in a biological sample obtained from the subject as compared to a reference level, wherein such elevated level is indicative the subject has an increased risk of developing neurodegenerative disease. In certain embodiments, the method further includes identifying a subject having an increased level of permeability of the gut barrier as compared to a reference level (e.g., a level of permeability of the gut barrier in a healthy subject) as having an increased risk of developing neurodegenerative disease.

Methods for identifying the presence of, and quantifying the amount of, CML, a CML precursor, a CML metabolite, or a CML analog can include analytical techniques known in the art including for example, chromatography (e.g., high performance liquid chromatography (HPLC)), mass-spectroscopy, liquid chromatography-mass spectrometry (LC-MS), nuclear magnetic resonance spectroscopy, or immunoassay.

For example, the amount of CML, a CML precursor, a CML metabolite, or a CML analog may be detected and/or quantified in a tissue or body fluid sample by one or more, chromatographic methods, mass spectrometry (MS) methods, chromatographic methods followed by MS, electrophoretic methods, electrophoretic methods followed by MS, nuclear magnetic resonance (NMR) methods, and combinations thereof. Exemplary chromatographic methods include, but are not limited to, Strong Anion Exchange chromatography using Pulsed Amperometric Detection (SAX-PAD), liquid chromatography (LC), high performance liquid chromatography (HPLC), ultra-performance liquid chromatography (UPLC), thin layer chromatography (TLC), amide column chromatography, and combinations thereof. Exemplary mass spectrometry (MS) include, but are not limited to, tandem MS, LC-MS, LC-MS/MS, matrix assisted laser desorption ionization mass spectrometry (MALDI-MS), Fourier transform mass spectrometry (FTMS), ion mobility separation with mass spectrometry (IMS-MS), electron transfer dissociation (ETD-MS), Multiple Reaction Monitoring (MRM), and combinations thereof. Exemplary electrophoretic methods include, but are not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof. Exemplary nuclear magnetic resonance (NMR) include, but are not limited to, one-dimensional NMR (1 D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NM R), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof. Any method of MS known in the art may be used to determine, detect, and/or measure CML, a CML precursor, a CML metabolite, or a CML analog of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature (see, Yates et al., (2009) ANNU. REV. BIOMED ENG. 11:49-79).

Exemplary immunoassays include, without limitation, immunohistochemical and/or western blot analysis, immunoprecipitation, enzyme linked immunosorbent assays (ELISA), enzyme-linked immunofiltration assay (ELIFA).

For example, in some embodiments, the sample may be contacted with an antibody specific for the target analyte (e.g., CML) under conditions sufficient for an antibody-target complex to form, and detection of the complex. The presence of the analyte may be detected in a number of ways, such as by Western blotting or ELISA procedures using any of a wide variety of tissues or samples, including plasma or serum. A wide range of immunoassay techniques using such an assay format are available, see, e.g., U.S. Pat. Nos. 4,016,043, 4,424,279, and 4,018,653. These include both single-site and two-site or “sandwich” assays of the noncompetitive types, as well as traditional competitive binding assays. These assays also include direct binding of a labeled antibody to a target analyte. The resulting complexes can be detected by the signal emitted by a label, e.g., an enzyme, a fluorescent label, a chromogenic label, a radionuclide containing molecule (i.e., a radioisotope), or a chemiluminescent molecule.

In various embodiments, CML is detected with an anti-CML antibody, such as an anti-CML antibody from Immunochem (Catalog Number ICP2188), Creative Diagnostics (Catalog Number DMABT-Z59348), Creative BioLabs (Catalog Number AGM-233YJ), Hycult Biotech (Catalog Number HM5013), Abcam (Catalog Number ab125145, ab27683, ab27685, ab27684, or ab30922), MyBioSource (Catalog Number MBS390033 or MBS390034), Kerafast (Catalog Number EMS302), and Biotechne (Catalog Number MAB3247-SP or MAB3247), or any now known or later identified anti-CML antibody.

In various embodiments, CML is detected with a CGYJ107, CML26, 6C7, MAB3247, or CMS-10 anti-CML antibody clone.

In various embodiments, a CML analog, such as N(6)-(1-carboxyethyl)-L-lysine (CEL) is detected with an anti-CEL antibody, such as an anti-CEL antibody from Abcam (Catalog Number ab145095) or Cosmo Bio (Catalog Number CAC-AGE-M02). In various embodiments, CEL is detected with a CEL-SP anti-CEL antibody clone.

In alternative methods, the expression of CML, a CML precursor, a CML metabolite, or a CML analog in a sample may be examined using immunohistochemistry (“IHC”) and staining protocols. IHC staining of tissue sections has been shown to be a reliable method of assessing or detecting presence of a target in a sample. IHC and immunofluorescence techniques use an antibody to probe and visualize cellular antigens in situ, generally by chromogenic or fluorescent methods. The tissue sample may be fixed (i.e., preserved) by conventional methodology (see, e.g., “Manual of Histological Staining Method of the Armed Forces Institute of Pathology,” 3rd edition (1960) Lee G. Luna, H T (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). Generally, the sample is first fixed and is then dehydrated through an ascending series of alcohols, infiltrated and embedded with paraffin or other sectioning media so that the tissue sample may be sectioned. Alternatively, one may section the tissue and fix the sections obtained. The primary and/or secondary antibody used for immunohistochemistry typically will be labeled with a detectable moiety, such as a radioisotope, a colloidal gold particle, a fluorescent label, a chromogenic label, or an enzyme-substrate label.

### IV. Pharmaceutical Compositions, Medicaments and Routes of Administration

The methods described herein use pharmaceutical compositions or medicaments comprising one or more gut barrier function enhancers and/or agents for reducing or eliminating gut microbiota dysbiosis, or pharmaceutically acceptable salts or solvates thereof, and at least one pharmaceutically acceptable carrier.

Gut barrier function enhancers can be used to reduce the concentration of CML, a CML precursor, a CML metabolite, or a CML analog in a tissue or body fluid of a subject. Exemplary gut barrier function enhancers include, for example, intestinal alkaline phosphatase (IAP) (exemplary CAS No. 9001-78-9 (calf)), a polyphenol (e.g., ellagic acid (EA) (exemplary CAS No. 476-66-4) or lipoteichoic acid), metformin (exemplary CAS No. 657-24-9), urolithin A (exemplary CAS No. 1143-70-0), butyrate (exemplary CAS No. 156-54-7), glutamine (exemplary CAS No. 56-85-9), obeticholic acid (OCA) (exemplary CAS No. 459789-99-2), divertin, or curcumin (exemplary CAS No. 458-37-7), or derivatives thereof.

Agents for reducing or eliminating gut microbiota dysbiosis can be used to reduce the concentration of CML, a CML precursor, a CML metabolite, or a CML analog in a subject. Exemplary agents for reducing or eliminating gut microbiota dysbiosis include, for example, IAP, EA, biotics, probiotics (e.g., Biohm Probiotic Boost and Biohm Colon Cleanse from Biohm Health), prebiotics and postbiotics. Exemplary probiotics include bacteria belonging to genera Lactobacillus (e.g., Lactobacillus acidophilus or Lactobacillus rhamnosus), Bifidobacterium (e.g., Bifidobacterium breve), Saccharomyces (e.g., Saccharomyces cerevisae) and Streptococcus (e.g., Streptococcus thermophilus). Prebiotics enhance the growth of specific beneficial bacterial species that elicit health benefits, and exemplary prebiotics include lipoteichoic acid and polyphenols. Postbiotics are, for example, metabolic products, fermentation products, minerals (e.g., zinc and selenium), microelements, micronutrients, cell surface proteins, and organic acids generated by the microbiome during its life cycle.

In accordance with the methods described herein, the described gut barrier function enhancers and/or agents for reducing or eliminating gut microbiota dysbiosis or salts, solvates, or prodrugs thereof may be administered to a subject in a variety of forms depending on the selected route of administration. Accordingly, the compositions described herein may be formulated for administration, for example, by oral, parenteral, administration, and the pharmaceutical compositions formulated accordingly. Parenteral administration includes intravenous, intraperitoneal, subcutaneous, intramuscular, transepithelial, nasal, intrapulmonary, intrathecal, intracerebroventricular, intraparenchymal, rectal, and topical modes of administration.

According to a particular embodiment, the pharmaceutically acceptable carrier of a composition useful in the practice of the invention is formulated for oral administration or intravenous administration.

The pharmaceutical compositions containing compound described herein may be manufactured in a manner that is generally known, e.g., by means of conventional mixing, dissolving, granulating, levigating, emulsifying, encapsulating, entrapping, or lyophilizing processes. Pharmaceutical compositions may be formulated in a conventional manner using one or more pharmaceutically acceptable carriers including excipients and/or auxiliaries that facilitate processing of the compounds into preparations that can be used pharmaceutically. It is appreciated that the appropriate formulation is dependent upon the route of administration chosen.

Oral compositions generally include an inert diluent or an edible pharmaceutically acceptable carrier. They can be enclosed in gelatin capsules or compressed into tablets. For the purpose of oral therapeutic administration, the compounds described herein can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, an active ingredient (e.g., a gut barrier function enhancer and/or an agent for reducing or eliminating gut microbiota dysbiosis); a binder such as microcrystalline cellulose, gum tragacanth or gelatin; an excipient such as starch or lactose, a disintegrating agent such as alginic acid, Primogel, or corn starch: a lubricant such as magnesium stearate; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, non-limiting suitable carriers include physiological saline, bacteriostatic water, or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

For administration by inhalation, the gut barrier function enhancers are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, and bile salts. Transmucosal administration can be accomplished through the use of nasal sprays or suppositories. For transdermal administration, the active gut barrier function enhancers is formulated into ointments, salves, gels, or creams as generally known in the art.

The active gut barrier function enhancers can be prepared with pharmaceutically acceptable carriers that will protect the gut barrier function enhancer against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active gut barrier function enhancer calculated to produce the desired therapeutic effect in association with the required pharmaceutical carrier. The specification for the dosage unit forms are dictated by and directly dependent on the unique characteristics of the active gut barrier function enhancer and the particular therapeutic effect to be achieved.

In therapeutic applications, the dosages of the pharmaceutical compositions used in accordance with the disclosure vary depending on the agent, the age, weight, and clinical condition of the recipient subject, and the experience and judgment of the clinician or practitioner administering the therapy, among other factors affecting the selected dosage. Generally, the dose should be sufficient to result in slowing, and preferably regressing, the symptoms of the disease or disorder disclosed herein and preferably causing complete regression of the disease or disorder. An effective amount of a pharmaceutical agent is that which provides an objectively identifiable improvement as noted by the clinician or other qualified observer.

It is to be understood that the pharmaceutical compositions can be included in a container, pack, or dispenser together with instructions for administration.

### V. Methods for Screening for Gut Barrier Function Enhancers

Also provided herein are methods of screening for a candidate agent for decreasing the rate of accumulation of CML, a CML precursor, a CML metabolite, or a CML analog in a tissue or body fluid sample of a subject that include: determining a first level of CML, a CML precursor, a CML metabolite, or a CML analog in a biological sample obtained in a mammal at a first time point; administering an agent to the subject; and determining a second level of CML, the CML precursor, or the CML metabolite in a biological sample obtained in a mammal at a second time point; wherein an agent that results in a reduction in the second level as compared to the first level is identified as a candidate agent for decreasing the rate of accumulation of CML, a CML precursor, a CML metabolite, or a CML analog in a tissue of a subject.

Also provided herein are methods of screening for a candidate agent for decreasing the rate of development of oxidative or metabolic stress in microglia in a subject that include: determining a first level of CML, a CML precursor, a CML metabolite, or a CML analog in a biological sample obtained in a mammal at a first time point; administering an agent to the subject; and determining a second level of CML, the CML analog, the CML precursor, or the CML metabolite in a biological sample obtained in a mammal at a second time point; wherein an agent that results in a reduction in the second level as compared to the first level is identified as a candidate agent for decreasing the rate of development of oxidative or metabolic stress in microglia in a subject.

Also provided herein are methods of screening for a candidate agent for decreasing the rate of development of mitochondrial dysfunction in microglia in a subject that include: determining a first level of CML, a CML precursor, a CML metabolite, or a CML analog in a biological sample obtained in a mammal at a first time point; administering an agent to the subject; and determining a second level of CML, the CML precursor, the CML metabolite, or the CML analog in a biological sample obtained in a mammal at a second time point; wherein an agent that results in a reduction in the second level as compared to the first level is identified as a candidate agent for decreasing the rate of development of mitochondrial dysfunction in microglia in a subject.

Also provided herein are methods of screening for a candidate agent for decreasing the rate of development of microglial dysfunction in a subject that include: determining a first level of CML, a CML precursor, a CML metabolite, or a CML analog in a biological sample obtained in a mammal at a first time point; administering an agent to the subject; and determining a second level of CML, the CML precursor, the CML metabolite, or the CML analog in a biological sample obtained in a mammal at a second time point; wherein an agent that results in a reduction in the second level as compared to the first level is identified as a candidate agent for decreasing the rate of development of microglial dysfunction in a subject.

Also provided herein are methods of screening for a candidate agent for increasing one or more functions of microglia in a subject that include: determining a first level of CML, a CML precursor, a CML metabolite, or a CML analog in a biological sample obtained in a mammal at a first time point; administering an agent to the subject; and determining a second level of CML, the CML precursor, the CML metabolite, or the CML analog in a biological sample obtained in a mammal at a second time point; wherein an agent that results in a reduction in the second level as compared to the first level is identified as a candidate agent for increasing one or more functions of microglia in a subject.

Also provided herein are methods of screening for a candidate agent for decreasing the rate of development or worsening of cognitive impairment in a subject that include: determining a first level of CML, a CML precursor, a CML metabolite, or a CML analog in a biological sample obtained in a mammal at a first time point; administering an agent to the subject; and determining a second level of CML, the CML precursor, the CML metabolite, the CML analog in a biological sample obtained in a mammal at a second time point; wherein an agent that results in a reduction in the second level as compared to the first level is identified as a candidate agent for decreasing the rate of development or worsening of cognitive impairment in a subject.

In certain embodiments, the amount of CML, a CML precursor, a CML metabolite, or a CML analog in a biological sample is measured. Methods for measuring the amounts of such agents are described above in Section III and include, for example, chromatography (e.g., high performance liquid chromatography (HPLC)), mass-spectroscopy, liquid chromatography-mass spectrometry (LC-MS), nuclear magnetic resonance spectroscopy, or immunoassay.

In certain embodiments of these methods of screening, the method further includes testing the candidate agent in an animal model.

### EXAMPLES

Below are examples of specific embodiments for carrying out the present invention. The examples are offered for illustrative purposes only and are not intended to limit the scope of the present invention in any way.

### Example 1. Gut Microbiota Drives Age-Related Oxidative Stress and Mitochondrial Damage in Microglia Via N6-Carboxymethyllysine (CML)

This Example describes the discovery of CML as a messenger for communicating between the gut microbiota and the brain in young-adult and aged mice.

Microglial transcriptomes from young-adult and aged mice housed under germ-free and specific pathogen-free conditions were compared and it was found that the microbiota influenced aging associated-changes in microglial gene expression. The absence of gut microbiota diminished oxidative stress and ameliorated mitochondrial dysfunction in microglia from the brains of aged mice. Unbiased metabolomic analyses of serum and brain tissue revealed the accumulation of N6-carboxymethyllysine (CML) in the microglia of the aging brain. CML mediated a burst of reactive oxygen species and impeded mitochondrial activity and ATP levels in microglia. The age-dependent rise in CML levels in the sera and brains of humans was validated, and it was found that a microbiota-dependent increases in intestinal permeability in aged mice mediated the elevated levels of CML. These results demonstrate how the gut microbiota influences the homeostasis of microglia in the aging brain and molecular phenotyping at the metabolite level identified CML, a major advanced glycation end product (AGE), as a key compound responsible for age-related microglial dysfunction.

### Methods

### Human Tissue

Formalin-fixed paraffin-embedded (FFPE) cortical tissue from healthy brains were examined by a fully trained neuropathologist (control tissue or Braak stages I and II) from 43 individuals (20 females and 23 males aged 1-88 years; 8 temporal, 25 frontal lobe) at the Institute of Neuropathology, University Hospital Freiburg, Germany.

### Mice

Specific pathogen-free (SPF) and germ-free (GF) housed C57BL/6 mice were analyzed at 6-10 weeks of age (young-adult) and 96-104 weeks of age (aged). Mice of the aged group in the nontargeted metabolomics analysis were 17-18 months old. Except for nontargeted metabolomics and microbiota profiling, where only male mice were used, all groups included mice from both sexes. Mice were housed under a 12-hour light/12-hour dark cycle and temperatures of 18-23° C. with 40-60% humidity, with food and water given ad libitum. To avoid cage effects, mice from at least three different cages per experimental group were analyzed. For the treatment, young-adult mice (8 weeks old) were injected intraperitoneally or by oral gavage with CML (0.735 mg kg⁻¹; Iris Biotech), trimethylamine N-oxide (TMAO) (3.95 mg kg⁻¹; Sigma-Aldrich), sodium acetate (59 mg kg⁻¹; Sigma-Aldrich) and sodium propionate (4.61 mg kg⁻¹; Sigma-Aldrich) daily for 14 days. For modulation of CML in aged animals, 18-month-old SPF housed C57BL/6 mice were treated orally every third day for 10 weeks with vehicle (20% hydroxypropyl-p-cyclodextrin in 1× phosphate-buffered saline (PBS)), 10 mg kg⁻¹ ellagic acid (EA) or 3,000 U kg⁻¹ intestinal alkaline phosphatase (IAP). To assess in vivo intestinal permeability, tracer fluorescein isothiocyanate (FITC)-labeled dextran (4 kDa: Sigma-Aldrich) was used. Briefly, mice were deprived of food 4 hours before and both food and water 4 hours after oral gavage using 200 μl of 80 mg ml⁻¹ FITC-dextran. Blood was collected retro-orbitally after 4 hours and fluorescence intensity was measured on fluorescence plates using an excitation wavelength of 493 nm and an emission wavelength of 518 nm. To assess in vivo intestinal permeability to CML, mice were treated with 200 pl CML (0.36 mM). Blood was collected retro-orbitally immediately before and 4 hours after treatment. CML translocated into the circulation was measured by liquid chromatography-mass spectrometry (LC-MS). All animal experiments were approved by the local administration in Germany (Regierungsprasidium Freiburg) and were performed in accordance with the respective national, federal, and institutional regulations and the guidelines of the Federation of European Laboratory Animal Science Associations.

### Preparation of Mouse Tissue Samples

Mice were lethally anesthetized with ketamine (100 mg kg⁻¹ body weight) and xylazine (10 mg kg⁻¹ body weight) followed by perfusion with 1×PBS through the left heart chamber. For histology, brains were kept overnight in 4% paraformaldehyde (PFA). For flow cytometry and magnetic-activated cell sorting (MACS) beads cell sorting, brains were dissected, homogenized and filtered through a 70-pm mesh. After centrifugation (220 g for 5 minutes at 4° C.), pellets were resuspended in 37% Percoll followed by centrifugation for 30 min, 800 g at 4° C. Myelin was removed from the top layer and the cell pellet was washed once with 1×PBS followed by antibody staining.

### Immunohistochemistry

Brains were fixed in 4% PFA overnight and embedded in paraffin. Three micrometer-thick parasagittal sections were stained with anti-Iba-1 antibody (1:500 dilution, catalog no. 019-19741; WAKO) to assess microglial cell density. For immunohistochemistry (IHC), epitopes were unmasked by heat-induced antigen retrieval at pH 6. The primary antibody was incubated overnight (4° C.) followed by incubation with biotin-labeled goat anti-rabbit secondary antibodies (1:1,000 dilution; SouthernBiotech) for 45 minutes at room temperature. Streptavidin-horseradish peroxidase (SouthernBiotech) was then added for 45 minutes at room temperature. 3,3′-Diaminobenzidine brown chromogen (Dako) was used to resolve the antibody's signal. Nuclei were counterstained with hematoxylin. Images were acquired with the BZ-9000 Biorevo microscope (Keyence) and analyzed with the ImageJ v. 1.53f (National Institutes of Health) software.

### Immunofluorescence

Brains were fixed in 4% PFA, dehydrated in 30% sucrose and embedded in Tissue-Tek OCT compound (Sakura Finetek). Fourteen micrometer-thick cryosections were obtained using a cryostat (SM2000R; Leica Biosystems). FFPE tissue from mouse and human brains was sectioned by a microtome to obtain 5-pm sections. Epitopes were unmasked by heat-induced antigen retrieval at pH 6. Sections were blocked with PBS containing 5% bovine serum albumin (BSA) and permeabilized with 0.5% Triton X-100 in blocking solution. The following primary antibodies were incubated overnight at 4° C.: rabbit anti-Iba-1 (1:500 dilution; WAKO); guinea pig anti-Iba-1 (1:1,000 dilution; Synaptic Systems); anti-iNOS (1:500 dilution; Thermo Fisher Scientific) or anti-CML (1:500; Abcam). Secondary antibodies (Alexa Fluor 488-, 568- or 647-conjugated, 1:500 dilution) were incubated for 2 hours at room temperature. For three dimensional (3D)-reconstruction of microglia, free-floating 30-pm cryosections from brain tissue were labeled for 48 hours with anti-Iba-1 (1:500 dilution) at 4° C., followed by Alexa Fluor 647-conjugated secondary antibody at a dilution of 1:500 overnight at 4° C. Slides were treated with TrueBlack Lipofuscin Autofluorescence Quencher to eliminate autofluorescence in tissue from aged mice and human individuals. Nuclei were counterstained with 4′,6-diamidino-2-phenylindole (DAPI). Coverslips were mounted with ProLong Diamond Antifade Mountant (Thermo Fisher Scientific). Images were acquired using a BZ-9000 Biorevo microscope using a 20×/0.75 numerical aperture (NA) objective, an Olympus Fluoview 1000 confocal laser scanning microscope or with a TCS SP8 X (Leica Microsystems) using a 20×/0.75 NA objective (HC PL APO 20×/0.75 NA IMM CORR CS2). For 3D-reconstruction of microglia, images were analyzed using Imaris v.8.02 (Bitplane) with at least five cells per mouse. All other images were processed and analyzed with Photoshop CC 2015 (Adobe) or ImageJ v.1.53f (National Institutes of Health).

### Electron Microscopy

Brain specimens from the cortex were first fixed overnight in 3% glutaraldehyde at 4° C., washed with Sorensen buffer, and then transferred to 1% osmium tetroxide for 2 hours at room temperature. Next, samples were dehydrated by a graded series of ethanol (30-100%) followed by 100% propylene oxide, resin/propylene oxide (1:2 (v/v)) and resin/propylene oxide (2:1 (v/v)). Samples were embedded in resin via polymerization for 24 hours at 75° C. Then, 700-nm semithin sections were cut and stained with 2% toluidine blue to define the region of interest for further preparation of 70 nm ultrathin sections using an ultramicrotome (Leica Reichert Ultracut S) after contrasting the sections with uranyl acetate and lead citrate (Leica Reichert Ultrastainer). To assess the mitochondrial phenotype in microglia, images were acquired with a ×7,900 or ×46,000 magnification using a CM100 electron microscope (Philips). Images of 30-35 cells per mouse were processed and analyzed with the iTEM software 2012 (Olympus).

### Bone Marrow Derived Macrophages Cell Culture

Cells were cultured at 37° C. and 5% CO₂ in a humidified incubator. Murine BMDMs were differentiated from tibial and femoral bone marrow aspirates. Recombinant murine macrophage colony-stimulating factor (Immunotools) was used at 20 ng ml⁻¹. After 7 days of differentiation, BMDMs were seeded in 24-well plates with 5×10⁵ cells per well in triplicates. The medium was switched to serum-free medium 6 hours before the experiment. Cells were incubated with increasing concentrations of CML (untreated, 0.1 pM, 1 pM, 10 pM, 100 pM, 1 mM) for 48 hours, then collected for measurements.

### Flow Cytometry

Cell sorting for RT-qPCR and RNA sequencing (RNA-seq) was performed on a MoFlo Astrios (Beckman Coulter; FIG. 7a). Before surface staining, dead cells were excluded by using the Fixable Viability Dye eFluor 780 (1:1,000 dilution; Thermo Fisher Scientific) followed by incubation with Fc receptor blocking antibody CD16/CD32 (1:200 dilution, clone 2.4G2; BD Bioscience). The following antibodies were used for surface staining: anti-CD45 (1:200 dilution, clone 30-F11; Thermo Fisher Scientific); anti-CD11b (1:200 dilution, clone MI/70; Thermo Fisher Scientific). The following lineage antibodies were used (all at 1:300 dilution): anti-CD3 (clone 17A2; BioLegend); anti-CD19 (clone 6D5; BioLegend); anti-CD45R (clone RA3-6B2; BD Biosciences); Ly6C (clone AL-21; BD Biosciences); Ly6G (clone 1A8; BD Biosciences). To assess microglial cellular reactive oxidative species (ROS), the CellROX DeepRed Reagent (5 pM; Thermo Fisher Scientific) was used. To assess mitochondrial activity, Tetramethylrhodamine, Methyl Ester, Perchlorate (50 nM; Thermo Fisher Scientific) and MitoTracker Green FM (20 nM; Thermo Fisher Scientific) were used. Dead cells were excluded by short incubation with DAPI before flow cytometry analysis with the FACSCanto II (BD Biosciences). Data were acquired with the FACSDiva v.6 software (Becton Dickinson). Postacquisition analysis was performed with FlowJo v.10 (FlowJo LLC).

### Cellular ATP Measurement

To avoid the cellular stress of fluorescence-activated cell sorting (FACS), microglial cells were isolated using the magnetic-activated cell sorting (MACS) separation system (Miltenyi Biotec; FIG. 7b). The cell suspension was incubated with Fc receptor blocking antibody CD16/CD32 (clone 2.4G2; BD Biosciences) and with biotinylated anti-CD11b antibody (clone M1/70; Thermo Fisher Scientific). Anti-biotin microbeads (Miltenyi Biotec) were then added to the cell suspension and positive selection was carried out according to the manufacturer's instructions. From each sample, 10,000 cells per well were plated in 96-well plates in triplicates. Cellular ATP was measured using the CellTiter-Glo assay (Promega Corporation) according to the manufacturer's instructions.

### RNA-Seq

Total RNA was extracted from FACS-sorted CD11b⁺CD45^{int}Lin⁺ microglia (10,000 cells per sample) using the Arcturus PicoPure RNA Isolation Kit (Thermo Fisher Scientific) according to the manufacturer's protocol. The SMARTer v4 Ultra Low Input RNA Kit for Sequencing (Clontech) was used to generate first-stranded complementary DNA. Double-stranded cDNA was amplified by long-distance PCR (11 cycles) and purified via magnetic bead cleanup. Library preparation was carried out as described in the Illumina Nextera XT sample preparation guide (illumina). The sequencing run was performed on a HiSeq 1000 instrument (Illumina) using the indexed, 50-cycle single-read protocol and the TruSeq SBS v3 Reagents according to the HiSeq 1000 system user guide. BCL files were converted into FASTQ files with the CASAVA1.8.2 software. Library preparation and RNA-seq were performed at the Genomics Core Facility ‘Center of Excellence for Fluorescent Bioanalytics’, University of Regensburg, Germany.

The quality of sequencing reads stored in the FASTQ files was assessed with FastQC v.0.67 and trimmed with Trim Galore! v.0.4.3. Reads were mapped on the mouse genome version mm10 (University of California Santa Cruz) using STAR aligner v.2.5.2 with RefGene annotation. The number of reads mapped to each gene (counts) were extracted from the BAM files using FeatureCount v.1.5.3. The process to extract the gene counts from FASTQ files was run on the Galaxy platform (Afgan et al. (2018) NUCLEIC ACIDS RES., 46: W537-W544). Three samples with a low mapping rate (<75%) were removed.

Differential expression analysis was performed with DESeq2 v.1.32.0. Normalized counts generated by DESeq2 were assessed for artifacts or contamination by other cell types. The list of genes used was based on single-cell RNA-seq data (Jordao et al. (2019) SCIENCE, 363: eaat7554). R v.4.1.0 was used to perform the Ward error sum of squares hierarchical clustering method and PCA. Using the DESeq2 model, differentially expressed genes (DEGs) with adjusted P<0.05 (Wald test) and absolute fold change >1.5 were identified. Heatmaps were plotted using the R package pheatmap v.1.0.8 calculated from scaled (z-scores) normalized read counts of DEGs with a hierarchical clustering of the rows (complete method). A WGCNA was performed on normalized expression data using the R package WGCNA v.1.69. For computational efficiency, genes were filtered to keep only genes that explained more than 50% of the variance (10,848 kept genes and 7,265 removed). A module-trait correlation analysis was performed between the module eigengenes (ME) and the different traits (combination of microbiota and age) by computing the Pearson correlation between each pair of variables and Student asymptotic P values for the correlations using the WGCNA package. A Gene Ontology (GO) enrichment analysis of the genes in the different MEs was made using goseq v.1.44.0, with the genome-wide annotation for Mouse (org.Mm.eg.db v.3.13.0) and Wallenius approximation.

The overenriched GO categories were extracted using a 0.05 false discovery rate (FDR) cutoff. The lists of ROS-related genes were extracted from GO:0000302, GO:2000377 and oxidative stress (WikiPathways).

### RT-qPCR

Hif1a gene expression was measured using the TaqMan assay (Mm00468869_m1). Data were normalized to the value obtained from the microglia of the young-adult SPF male group at homeostasis and relative gene expression levels were determined by the ΔΔCT method. Gene expression was considered undetectable if the CT values were >35 cycles.

### Microbiome Profiling

Total DNA was isolated from fecal samples using the QIAamp DNA stool kit (QIAGEN) according to the modified manufacturer's instructions (Yilmaz et al. (2019) NAT. MED., 25: 323-336). Briefly, 100-200 mg were homogenized in 500 pl ASL buffer by the bead-beating step using TissueLyser for 3 minutes at 30 Hz followed by two additional lysis steps at 95° C. Afterwards, samples were incubated with 200 pl lysis buffer for Gram+ bacteria (20 mg ml⁻¹ lysozyme, 20 mM Tris-HCl, pH 8.0, 2 mM EDTA, 1.2% Triton; Sigma-Aldrich). DNA was purified and pooled at a concentration of 26 pM and the pooled library was sequenced for the V5/V6 region of 16S rRNA genes in an IonTorrent PGM system according to the manufacturer's instructions (Thermo Fisher Scientific).

An average of 38,209 high-quality reads per sample were used for microbiome profiling. Reads were clustered in operational taxonomic units (OTUs) at 97% of similarity. Data were further analyzed using the QIIME v.1.9.1 pipeline after filtering out low-quality (accuracy of base calling; q<25) samples; samples with >4,500 reads were retained for further analysis (Caporaso et a/. (2010) NAT. METHODS, 7: 335-336). OTUs were chosen using UCLUST with a 97% sequence identity threshold followed by taxonomy assignment using the SILVA database release 119. Alpha and Beta diversity were calculated using the phyloseq pipeline in R v.3.4. The nonparametric Mann-Whitney U-tests were used to compare Alpha diversity between samples and Adonis from the vegan R package v2.5-7 to assess the effects of groups for Beta diversity via phyloseq (McMurdie et al. (2012) PAC. SYMP. BIOCOMPUT., 235-246; Callahan et al (2016) F1000RES., 5: 1492). Taxonomic differences at the phylum and genus levels between tested groups were identified using the ‘multivariate analysis by linear models’ R package v0.0.4. Plots were generated with ggplot2 v.3.3.5 using a phyloseq object. Only taxa present in at least 30% of samples and OTUs including more than 0.0001% of total counts were considered. A P<0.05 and an FDR of q<0.05 (with Benjamini-Hochberg correction) were used as cutoff values for significance.

### Nontargeted Metabolomics

Raw data on serum and brain of young-adult and aged mice groups were mined. Nontargeted MS analysis was performed at Metabolon (Evans et al. (2009) ANAL. CHEM., 81: 6656-6667). Peaks were quantified using the area under the curve. Raw area counts for each metabolite in each sample were normalized to correct for variation resulting from instrument interday tuning differences by the median value for each run day, thus setting the medians to 1.0 for each run. This preserved variation between samples but allowed metabolites of widely different raw peak areas to be compared on a similar graphical scale. Missing values were imputed with the observed minimum after normalization.

### Targeted Metabolomics by LC-MS

Samples were extracted with precooled (−80° C.) extraction solution (80:20 methanol LC-MS grade: Milli-Q H₂O). Targeted metabolite quantification by LC-MS was carried out using an Agilent 1290 Infinity II UHPLC system in line with an Agilent 6495 QQQ-MS operating in multiple reaction monitoring (MRM) mode. MRM settings were optimized separately for all compounds using pure standards. LC separation was conducted on a Phenomenex Luna propylamine column (50×2 mm, 3-μm particles) using a solvent gradient of 100% buffer B (5 mM ammonium carbonate in 90% acetonitrile) to 90% buffer A (10 mM NH₄ in water). Flow rate was from 1,000 to 750 pl min⁻¹. The autosampler temperature was 5 degrees and the injection volume was 2 pl. Peak areas were identified based on standards for each metabolite and calculated using MassHunter v.B.08.02 (Agilent). For SCFAs, namely acetate (C2, 59.04 g mol⁻¹), propionate (C3, 73.07 g mol⁻¹), butyrate (and isobutyrate, C4, 87), and valerate (and isovalerate, 101) were quantified in mouse serum. To extract the metabolites, 10 μl of each sample were added to 4 tubes, 90 μl of acetonitrile were added with serial dilution (4 levels) of standards. C2 (mg ml⁻¹) (L1:0; L2:0.002; L3:0.004; L4:0.006), C3 (mg ml⁻¹) (L1:0; L2:0.0002; L3:0.0004; L4:0.0006), C4 (mg ml⁻¹) (L1:0; L2:0.0005; L3:0.001; L4:0.0015) and C5(%) (L1:0; L2:0.0002; L3:0.0004; L4:0.0006), C4 (mg ml⁻¹) (L1:0; L2: 0.000025%; L3: 0.00005%; L4: 0.000075%). Samples were centrifuged at 20,000 g for 10 minutes at 4° C. and 50 μl of the supernatant were transferred to a new tube. For analysis by high-performance LC-quadrupole time of flight, 2 μl of each sample were injected. Peaks of butyrate and isobutyrate, and valerate and isovalerate, could not be robustly differentiated; therefore, for each pair, concentration values correspond to both moieties. Each sample was analyzed twice and the average value was used to build the regression line; the concentration was calculated using the standard addition method (Harris (2003) D. C. QUANTITATIVE CHEMICAL ANALYSIS 6TH EDN (W H Freeman and Co.)).

### Human Metabolomics Data

The TwinsUK adult twin registry includes about 14,000 individuals, predominantly females, with disease and lifestyle characteristic similar to the general UK population. The St. Thomas' Hospital Research Ethics Committee approved the studies and all twins provided informed written consent. Data were mined for CML and TMAO from a blood metabolome study that encompassed aging cohorts and was run on the Metabolon platforms. Briefly, metabolite ratios were measured in blood samples by Metabolon using an untargeted ultra-performance LC-MS/MS platform. Metabolites were scaled by run day medians and log-transformed.

### Statistics

No statistical methods were used to predetermine sample sizes. Data distribution was assumed to be normal. Wherever applicable, animals were randomly assigned to the different experimental groups. The experimenters were blind regarding group assignments. Statistical analyses, other than those of RNA-seq, nontargeted metabolomics and microbial profiling, were performed with Prism 9.0 (GraphPad Software).

### Results

### Gut Microbiota Alters the Microglial Transcriptomic Profile in Aging

A higher microglial cell density has previously been reported with aging in the brain cortex.

The increased microglial cell density in the cortex of specific pathogen-free (SPF) and germ-free (GF) animals between young-adult and aged mice was confirmed (see, FIGS. 6a-6b) but no difference between aged SPF and GF mice was found (see, FIGS. 6a-6b). To determine the potential morphological changes in microglia of SPF and GF mice, quantitative morphometric reconstruction was performed. The microglia of SPF mice displayed a reduction in total branch area, total branch length and the number of branch points, together with an increase in cell body volume (see, FIGS. 6c-6g). Cell body sphericity was not altered between the groups (see, FIG. 6h). These data demonstrate that age had an effect on microglial morphology in SPF mice, while microglia were unaltered and hyper-ramified under GF conditions.

To further evaluate the microbiota-dependent alterations of microglial physiology in the aging brain, RNA-seq was performed on FACS-purified microglia (see, FIGS. 7a and 8a) from the whole brains of young-adult (6-10-week-old) and aged (96-104-week-old) male and female mice, housed under GF or SPF conditions (see, FIG. 1b and FIG. 8b). PCA highlighted distinct gene expression profiles of microglia in GF and SPF mice across both age groups (see, FIG. 1c). The transcriptomic differences between microglia isolated from GF and SPF mice were more prominent at older ages (see, FIG. 1d). When compared to microglia from SPF mice, an age-independent gene expression pattern emerged in the microglia of GF mice (microglial GF signature), which included genes related to the cytoskeleton (for example, Sdc3, Sult1a1 and Tuba4a) and immune function (for example, Ctse, Ero1lb, Hira3, Kcnma1, Notch4, Nr1d2, Rab4a and Wdfy1) (see, FIG. 1e). Moreover, the microglial GF signature contained genes associated with the regulation of mitochondrial function (for example, B4galnt1, Gpr137b, Gstm1, Mcur1, Mtfp1, Nnt and Plcd3), which demonstrates a capacity of the microbiota to regulate the metabolic profile of microglia (see, FIG. 1e). Next, the functional changes in gene expression in microglia with regard to age and microbiota using a weighted gene coexpression network analysis (WGCNA) were characterized (see, FIGS. 1f-1g and FIG. 8c). Genes that significantly (Wald P_{adj}<0.05) explained more than 50% of the variance were binned into module eigengenes (MEs) based on their coexpression pattern. Upon comparing the young-adult groups under SPF or GF housing, minor differences in the gene networks associated with immune function and epigenetic regulation-ME1, ME5, ME6 and ME7, respectively, were observed. Two modules were characteristic of each age group. ME1 and ME4 in the aged SPF group included genes related to processes like mitochondrial metabolism and lipid localization, while ME2 and ME6 in the aged GF group included genes that regulate the immune response, histone lysine methylation and cell morphogenesis (see, FIG. 8d). ME1 and ME4 in the aged SPF group included genes related to processes like mitochondrial metabolism and lipid localization, while ME2 and ME6 in the aged GF group included genes that regulate regulation of immune response, histone lysine methylation and cell morphogenesis (FIG. 8c). Genes in ME1 & ME8, associated with immune response (e.g., Axl, Crlf2, Tnfsf8, Tnfsf10, Ccl12, Fgr, Il1b, Il6st, Spp1, and Tlr2), interferon signaling (e.g., Cxc10 8, Ifi207, Ifit2 8, and Stat1), inflammatory response (e.g., Cd180, Ldlr, S100a8, and S100a9), and microglial cell migration (e.g., Ccl12 and (Cxcl10) showed a specific upregulation in microglia of aged SPF mice, while having negative or low correlation in both young adult groups, and aged GF mice. ME1 and ME8, showed a strong correlation in the aged SPF group, including mitochondrial metabolic processes, hydrogen peroxide metabolic processes and reactive oxygen species metabolic processes. ME2, highly enriched in aged GF mice, was associated with response to oxygen-containing compounds (FIG. 8c). Genes in ME2, which include genes that regulate cellular ROS levels, such as Foxp1, Nrf1, and Trp53 and genes that are implicated in the regulation of mitochondrial ROS, such as G6pdx, Pdk2, Stat3 and Ucp2, were less expressed in the microglia of aged SPF mice when compared to age-matched GF mice, indicating that ROS-levels in aged SPF mice cannot be kept in an optimal cellular range. The accumulation of ROS in the aging brain is associated with mitochondrial damage and mitochondrial dysfunction. Indeed, in ME3, ME5 & ME9, we found prominent changes in genes related to mitochondrial assembly, carbohydrate metabolism, and oxidative phosphorylation. Although these genes were upregulated in SPF and GF mice, mitochondrial damage is more pronounced in SPF mice because here the protective ROS-regulating genes were downregulated. In addition, old GF mice upregulate genes, which are responsible for maintaining mitochondrial structure and function (FIG. 8d).

The microbiota's contribution to the age-related MEs was next examined. WGCNA revealed that microglia from aged GF mice followed the general aging trend in SPF mice, but with lower magnitude (see, FIGS. 1f-1g), and clustered closer to the young-adult groups (see, FIG. 8b). For example, genes in ME1 and ME8 associated with immune response (for example, Axl, Crfl2, Tnfsf8, Tnfsf10, Ccl12, Fgr, Il1b, Il6st, Spp1 and Tlr2), interferon signaling (for example, Cxcl10/8, Ifi207, Ift2/8 and Stat1), inflammatory response (for example, Cd180, Ldlr, S100a8 and S100a9) and microglial cell migration (for example, Ccl12 and Cxcl10) showed a specific upregulation in the microglia of aged SPF mice, while having negative or low correlation in both young-adult groups and aged GF mice (see, FIG. 2a and FIGS. 8c-8d). Microglia showed alterations in their transcriptomic profile with aging, with microbiota-dependent divergences.

### Reduced Oxidative Stress in the Microglia of Aged GF Mice

Upon inspecting the pathways in the age-related modules, several connections to the regulation of oxidative stress in microglia that were dependent on the microbiota were found. ME1 and ME8 showed a strong correlation in the aged SPF group, including mitochondrial metabolic processes, hydrogen peroxide metabolic processes and ROS metabolic processes. ME2, which was highly enriched in aged GF mice, was associated with response to oxygen-containing compounds (see, FIGS. 1f-1g and FIG. 2a). To confirm that the expression levels of microglial ROS-related genes were regulated by the age of the mice and housing condition, ROS-related genes in the age-related ME1, ME2 and ME8 were selectively analyzed (see, FIG. 2b). A specific upregulation of several immune activation and ROS-promoting genes, such as Cdkn1a, Cyba, Cybb, Duoxa1, Il1b, Tgfbr2, Tlr2, Tlr4 and Tlr5, and ROS response genes, such as Axl, Hif1a, Lcn2, Mmp2, Rela, Trex1, S100a8 and S100a9, only in the microglia of aged SPF mice were found (see, FIG. 2b). Genes in ME2, which included genes that regulate cellular ROS levels, such as Foxp1, Nrf1 and Trp53, and genes implicated in the regulation of mitochondrial ROS, such as G6pdx, Pdk2, Sta3 and Ucp2, were less expressed in the microglia of aged SPF mice compared to age-matched GF mice (see, FIG. 2b). ROS production in the microglia isolated from young-adult and aged SPF mice using the CellROX flow cytometry assay were monitored, and it was found that a significant elevation of ROS were observed with increasing age, which was reduced in aged GF mice (see, FIG. 2c). Activation of inducible nitric oxide synthase (iNOS) is directly linked to the generation of excessive ROS. Using immunohistochemistry (IHC), an age-dependent increase in microglial iNOS expression under SPF conditions was observed, which was less pronounced in GF mice (see, FIGS. 2d-2e).

To investigate how the increase in ROS might affect microglial function. ME3, ME5 and ME9 were observed. Changes in genes related to mitochondrial assembly, carbohydrate metabolism and oxidative phosphorylation were found (see, FIG. 2f). Electron microscopy examination of microglial mitochondria revealed a substantially higher percentage of damaged mitochondria with less well-defined or even destroyed cristae in aged SPF mice relative to aged GF mice, with no change in mitochondrial mass or number per microglia (see, FIGS. 2g-2h and FIGS. 9a-9c). The accumulation of cellular ROS, which peaked in the microglia of aged SPF mice, can induce the expression of hypoxia inducible factor 1 subunit Alpha (Hif1a). HIF1a is linked to ROS in pseudo-hypoxic states in the brain and can directly alter mitochondrial metabolism (Gomes et al. (2013) CELL, 155: 1624-1638). Mitochondrial dysfunction in the aged brain leads to a metabolic shift, which is associated with the exaggerated activation of microglia. While young-adult mice showed similar Hif1a expression, RNA-seq and quantitative PCR with reverse transcription (RT-qPCR) indicated higher Hif1a expression in the microglia from aged SPF mice than GF mice (see, FIGS. 9d-9e). The efficiency of oxidative phosphorylation declines in cells of aged animals and leads to reduced ATP production (Lopez-Otm et al. (2013) CELL, 153: 1194-1217). The transmembrane potential (ΔΨm) of mitochondria is the major driver of ATP production. To account for an increase in mitochondrial mass with age (see, FIGS. 9f-9g), mitochondrial activity was plotted as the transmembrane potential of mitochondria relative to the mitochondrial mass. The mitochondrial activity showed an age-associated drop and a reduction in the intracellular ATP reservoir in SPF mice, both of which were less pronounced in the microglia of GF mice (see, FIG. 2i, FIG. 7b, and FIG. 9h). Together, these data demonstrate that the microbiota contributes to increased oxidative stress in the microglia of the aged brain, which is associated with direct damage to the mitochondria.

Microbiota-Dependent Accumulation of CML with Age

The concentrations of short-chain fatty acids in the serum samples of young-adult and aged SPF mice were identified using targeted liquid chromatography-mass spectrometry (LC-MS) metabolite analysis (see, FIG. 3a). Acetate was the most abundant SCFA in sera of young adult and aged mice, and elevated concentrations of both acetate and propionate were found in the sera of aged mice compared to young-adult mice. Butyrate/isobutyrate and valerate/isovalerate were not changed (see, FIG. 3a). In an unbiased screening, a non-targeted metabolomics dataset was used and blood serum and brain samples from young-adult and aged mice housed under SPF conditions were studied (see, FIGS. 3b-3c). Pathway enrichment analysis revealed several tissue-specific pathway alterations. For example, pyrimidine, inositol, carnitine, and several pathways related to amino acid metabolism (e.g., lysine, polyamine and tyrosine) were more affected in the serum of aged mice (see, FIG. 10a). Vitamin A, tocopherol, purine metabolism, ceramide-related pathways, and the pentose phosphate pathway were specifically altered in the brains of aged mice (see, FIG. 10b). Pathways of fatty acid metabolism and advanced glycation end products (AGEs) were commonly altered in both serum and brain samples of aged mice (see, FIGS. 10a-10b). Metabolites that were significantly upregulated in both the serum and brain tissue of aged mice enabled identification of metabolites that were potentially regulated by the gut and reached the brain via the bloodstream. These metabolites included palmitoleate (16:1n7), TMAO, 1-oleoyl-2-docosahexaenoyl-glycerophosphorylcholine (18:1/22:6), CML and stachydrine (see, FIG. 3d). Selected age-related concentration changes seen in mice were confirmed in human blood sample. Non-targeted metabolomics on serum/plasma from a human aging cohort (TwinsUK data bank) recapitulated the age-related concentration changes of CML (see, FIG. 3e) and TMAO (see, FIG. 3f) as seen in mice. Targeted metabolomics from the brain tissue of young and aged SPF and GF mice demonstrated that a functional gut microbiota (e.g., in SPF mice) was necessary for the increase in CML and TMAO in aged mouse brain tissue. Aged GF mice displayed only minor changes compared to young GF mice (see, FIG. 3g).

### CML Enhances Age-Related Microglial Dysfunction

The functional effects of these metabolites on microglia in vivo was next evaluated. The following candidate metabolites were selected for further evaluation. CML, TMAO, acetate, and propionate. To identify the metabolite(s) responsible for the increased ROS production in aged microglia, each metabolite was administered separately to young-adult mice. To avoid potential artifacts related to different gut-to-circulation absorption profiles, young-adult mice were injected intraperitoneally once per day for two weeks with CML, TMAO, sodium acetate, or sodium propionate (see, FIG. 4a). TMAO, sodium acetate, and sodium propionate had no effect on intracellular ROS production or on the metabolic function of microglia. However, CML treatment could partially recapitulate the changes found in the microglia of aged mice.

CML increased oxidative stress, decreased metabolic activity and reduced cellular ATP stores (see, FIGS. 4b-4d). Moreover, CML caused mitochondrial dysfunction by directly inflicting damage to mitochondrial structures in the microglia (see, FIG. 10c). The impact of CML treatment was not only restricted to microglia but negatively impacted macrophages. Bone marrow-derived macrophages (BMDMs) showed a dose-dependent increase in oxidative stress and decreased metabolic activity in vitro (see, FIGS. 10d-10e). Circulating CML can be derived from an endogenous Maillard reaction, food or the conversion of AGEs by the gut microbiota (Tessier et al. (2016) MOL. NUTR. FOOD RES., 60: 2446-2456). Brain CML levels increased in aged SPF mice but not in aged GF mice (see, FIG. 4e), and CML was still detectable in the brain tissue of young-adult and aged GF mice to a similar level to that of young-adult SPF mice. These results demonstrate that the gut microbiota is required for increased CML levels in the aged brain but not for the baseline levels found in young-adult mice. Therefore, these results also demonstrate that the deregulation of mitochondrial function in microglia seen after intraperitoneal injection of CML resulted from increased brain CML concentrations, recapitulating the settings in aged brains (see, FIG. 4M). RNA-seq analysis of microglia showed that intraperitoneal injection of CML upregulated the expression of the ROS-related genes S100a9 and S100A8 and other microbiota- and aging-related genes, such as A430033K04Rik, Chic1, Ltf, Ngp, Pglyrp1, Scai and Zkscan2 (see, FIGS. 4g-4h and FIG. 10f).

Immunofluorescence staining of CML in the cortical microglia addressed whether microglia are directly targeted by CML, and showed that mice that were housed under SPF and GF conditions showed an increase in the percentage of CML⁺ microglia with age. Approximately 30% of microglia in aged SPF mice was CML⁺, and the microglia of aged GF mice showed less age-dependent accumulation of CML (see, FIGS. 4i-4j). Furthermore, the age-dependent increase in CML⁺ microglia seen in the mouse cortex was verified to also be present in the human cortex. Human brain tissues (total n=43: males=23, females=20) from individuals between the ages of 1 and 88 years were obtained, and a positive correlation (r=0.5793, R²=0.3356, P<0.001) between age and the percentage of CML⁺ microglia in the human cortex was observed (see, FIGS. 4k-4l). In mice, RNA-seq analysis of microglia indicated that i.p. injection of CML upregulated the expression of the ROS-related genes, S100a9 and S100A8, and other microbiota- and aging-related genes like A430033k04Rik, Chic1, Ltf, Ngp, Pglyrp1, Scai, and Zkscan2. These findings demonstrate that the age-related accumulation of CML induces microglial metabolic dysfunction in a direct fashion, including increased ROS, and may gradually disrupt brain homeostasis and brain function.

### Aged Microbiota Drives CML Levels by Disrupting the Gut-Blood Barrier

The age-dependent gut microbiota alterations by 16S ribosomal RNA-seq were characterized, based upon the finding that differences in CML levels and microglial function especially at an old age were dependent on the presence or absence of the microbiota. The distinctiveness of the microbiota profile of young-adult and aged mice was confirmed by Beta diversity analysis using the Bray-Curtis dissimilarity metric and the Shannon and Simpson Alpha diversity metrics (see, FIGS. 11a-11b). The gut microbiota in both age groups was dominated by two phyla (see, FIG. 11c), namely Firmicutes and Bacteroidetes. The relative abundance ratio of Firmicutes to Bacteroidetes is altered with advanced age in humans and can be linked to overall changes in bacterial profiles at different age stages (Mariat et al. (2009) BMC MICROBIOL., 9: 123). A significant age-dependent reduction in the Firmicutes to Bacteroidetes ratio was observed (Vaiserman et al. (2020) BMC MICROBIOL., 20: 221), where the phylum Firmicutes, family Lachnospiraceae was significantly diminished in aged mice (see, FIGS. 11d-11e). In the bacterial genera, an increased abundance of Turibacter, Alloprevotella, Parasutterella, Bifidobacterium, Macellibacteroides, Alistipes sensu stricto 1, Peptostreptococcaceae incertae sedis and Parabacteroides was observed in aged mice. This finding was in contrast to the abundance of Pantoea, Anoxybacillus, Lachnospiraceae incertae sedis, Cutrobacterium and Acetanfactor, which declined in aged mice (Hu et al. (2019) FOOD FUNCT., 10: 1736-1746) (see, FIG. 11_{f}). These findings demonstrate that profiling microbiota in young-adult and aged mice shows alterations at several taxonomic levels. Targeted metabolomics (LC-MS) measurements of CML in fecal pellets revealed that fecal pellets of aged GF mice had higher CML levels than those from aged SPF mice, demonstrating an indirect role of the microbiota in the age-related accumulation of CML in the brain (see, FIG. 5a).

Aged mice showed increased intestinal permeability compared to young-adult mice, a phenomenon that is dependent on the presence of the microbiota. Increased permeability allows metabolites to pass from inside the gastrointestinal tract through the intestinal epithelium more freely and enter the bloodstream, which could explain the discrepancy between CML levels in the brain and feces. To test this hypothesis, intestinal permeability was measured by quantifying the translocation of FITC-dextran (4 kDa) to the circulation after oral gavage. High gut permeability in aged SPF mice was observed, and the barrier function in aged GF mice was equivalent to that of young-adult SPF and GF mice (see, FIG. 5b). Colonization of young-adult GF mice with aged microbiota induced a microbiota-dependent increase in intestinal permeability compared to the permeability found after young-adult GF mice had received young gut microbiota (see, FIG. 5c). This aligned with the observation that the translocation of CML into the circulation after oral gavage was highest in aged SPF mice (see, FIG. 5d and FIG. 12a). To assess whether different routes of CML application would influence the accumulation of CML in microglia young-adult mice that had received CML intraperitoneally, rather than by oral gavage administration, were studied. Such mice showed more CML⁺ microglia in the cortex (see, FIGS. 12b-12c). In aged mice, the route of CML administration had no effect on the percentage of CML⁺ microglia (see, FIGS. 12b-12c). CML application by both intraperitoneal and oral gavage routes significantly exacerbated the age-related increase in cellular ROS and diminished metabolic function in microglia from aged mice. In young-adult mice, such an effect was only detectable after intraperitoneal administration of CML (see, FIGS. 12d-12e). To verify the key role of the gut barrier to the age-related accumulation of CML in microglia, aged SPF mice (18 months old) were treated every 3 days for 10 weeks orally with ellagic acid (EA), which prevents accumulation of CML, or intestinal alkaline phosphatase (IAP), an endogenous enhancer of the gut barrier function, by reducing the age-related microbiota dysbiosis and inducing autophagy in the gut epithelium (see, FIG. 5e). While EA had no effect on gut permeability, IAP-treated aged mice showed lower gut leakiness (see, FIG. 5f). Both EA and IAP reduced, either indirectly or directly, respectively, CML accumulation in the brain (see, FIG. 5g). The microglia of EA- and IAP-treated aged mice showed a significant reduction in cellular ROS and increased ATP levels compared to vehicle-treated aged mice (see, FIGS. 5h-5i). These findings demonstrate the impact of age-induced microbiota alterations, which disrupt the integrity of the gut barrier and facilitate the accumulation of CML in the brains of aged mice and humans (see, FIG. 1a).

### Example 2. Identification of Subject with Increased Risk of a Developing a Cognitive Disorder or a Neurodegenerative Disorder

A subject can be identified having, for example, an increased risk of developing microglial dysfunction, an increased risk of cognitive impairment or an increased risk of developing a neurodegenerative disease by a method comprising identifying a subject having an elevated level of CML, a CML precursor, a CML metabolite, or a CML analog in a biological sample obtained from the subject as compared to a reference level. The biological sample can be, for example, blood, serum, or plasma.

In some embodiments, the amount of CML, a CML precursor, a CML metabolite, or a CML analog in the biological sample is measured by a measured by chromatography (e.g., high performance liquid chromatography), mass-spectroscopy, liquid chromatography-mass spectrometry, or nuclear magnetic resonance spectroscopy. If an elevated level of CML, a CML precursor, a CML metabolite, or a CML analog in the biological sample is observed, the subject can be identified, for example, as having an increased risk of developing microglial dysfunction, an increased risk of cognitive impairment or an increased risk of developing a neurodegenerative disease.

### Embodiments

Described herein, in certain embodiments are methods of selecting a subject for treatment with a gut barrier function enhancer and/or an agent for reducing or eliminating gut microbiota dysbiosis, the method comprising: (a) identifying a subject having an elevated level of CML, a CML precursor, a CML breakdown product, or a CML metabolite in a biological sample obtained from the subject as compared to a reference level; and (b) selecting the identified subject for treatment with the gut barrier function enhancer and/or an agent for reducing or eliminating gut microbiota dysbiosis.

Described herein, in certain embodiments are methods of treating a subject comprising administering to the subject a therapeutically effective amount of a gut barrier function enhancer and/or an agent for reducing or eliminating gut microbiota dysbiosis to a subject identified as having an elevated level of CML, a CML precursor, a CML breakdown product, or a CML metabolite in a biological sample obtained from the subject as compared to a reference level.

Described herein, in certain embodiments are methods of decreasing the rate of accumulation of CML, a CML precursor, a CML breakdown product, or a CML metabolite in a tissue or body fluid sample of a subject, the method comprising administering to the subject a therapeutically effective amount of a gut barrier function enhancer and/or an agent for reducing or eliminating gut microbiota dysbiosis.

Described herein, in certain embodiments are methods of decreasing the rate of development of oxidative or metabolic stress in microglia in a subject in need thereof, the method comprising administering to the subject a therapeutically effective amount of a gut barrier function enhancer and/or an agent for reducing or eliminating gut microbiota dysbiosis.

Described herein, in certain embodiments are methods of decreasing the rate of development of mitochondrial dysfunction in microglia in a subject in need thereof, the method comprising administering to the subject a therapeutically effective amount of a gut barrier function enhancer and/or an agent for reducing or eliminating gut microbiota dysbiosis.

Described herein, in certain embodiments are methods of decreasing the rate of development of microglial dysfunction in a subject in need thereof, the method comprising administering to the subject a therapeutically effective amount of a gut barrier function enhancer and/or an agent for reducing or eliminating gut microbiota dysbiosis.

Described herein, in certain embodiments are methods of increasing one or more functions of microglia in a subject in need thereof, the method comprising administering to the subject a therapeutically effective amount of a gut barrier function enhancer and/or an agent for reducing or eliminating gut microbiota dysbiosis.

Described herein, in certain embodiments are methods of decreasing the rate of development or worsening of cognitive impairment in a subject in need thereof, the method comprising administering to the subject a therapeutically effective amount of a gut barrier function enhancer and/or an agent for reducing or eliminating gut microbiota dysbiosis.

Described herein, in certain embodiments are methods of treating cognitive impairment in a subject in need thereof, the method comprising administering to the subject a therapeutically effective amount of a gut barrier function enhancer and/or an agent for reducing or eliminating gut microbiota dysbiosis.

Described herein, in certain embodiments are methods of decreasing the rate of development or progression of a neurodegenerative disease in a subject, the method comprising administering to the subject a therapeutically effective amount of a gut barrier function enhancer and/or an agent for reducing or eliminating gut microbiota dysbiosis.

Described herein, in certain embodiments are methods of treating a neurodegenerative disease in a subject, the method comprising administering to the subject a therapeutically effective amount of a gut barrier function enhancer and/or an agent for reducing or eliminating gut microbiota dysbiosis.

Described herein, in certain embodiments are methods of decreasing the rate of development or worsening of neuronal dysfunction in a subject in need thereof, the method comprising administering to the subject a therapeutically effective amount of a gut barrier function enhancer and/or an agent for reducing or eliminating gut microbiota dysbiosis.

In certain embodiments, the subject has previously been identified as having an elevated level of CML, a CML precursor, a CML breakdown product, or a CML metabolite in a biological sample of a subject as compared to a reference level.

In certain embodiments, the method further comprises identifying the subject as having an elevated level of CML, a CML precursor, a CML breakdown product, or a CML metabolite in a biological sample of the subject as compared to a reference level.

In certain embodiments, the subject has previously been identified as having an elevated level of permeability of the gut barrier as compared to a reference level.

In certain embodiments, the method further comprises identifying the subject as having an elevated level of permeability of the gut barrier as compared to a reference level.

In certain embodiments, the biological sample comprises saliva, urine, blood, serum, plasma, cerebrospinal fluid, brain tissue, or feces.

In certain embodiments, the subject has been identified or diagnosed as having cognitive impairment.

In certain embodiments, the subject has been identified as having an increased risk of developing cognitive impairment.

In certain embodiments, the subject has been identified or diagnosed as having a neurodegenerative disease.

In certain embodiments, the subject has been identified as having an increased risk of developing neurodegenerative disease.

In certain embodiments, the method results in a reduction in level of cellular and/or mitochondrial ROS in microglia in the subject.

In certain embodiments, the method results in a reduction in expression of iNOS in microglia in the subject.

In certain embodiments, the method results in a reduction in expression of one or more genes in microglia of the subject selected from the group consisting of Cdkn1a, Cyba, Cybb, Duoxa1, Il1b, Tgfbr2, Tlr2, Tlr4, Tlr5, Axl, Hif1a, Lcn2, Mmp2, Rela, Trex1, S100a8, and S100a9.

In certain embodiments, the method results in an increase in expression of one or more genes in microglia of the subject selected from the group consisting of Foxp1, Nrf1, Trp53, G6pdx, Pdk2, Stat3, and Ucp2 in the subject.

In certain embodiments, the method further comprises determining the level of CML, CML precursor, CML breakdown product, or a CML metabolite in the biological sample obtained from the subject.

In certain embodiments, the neurodegenerative disease is selected from the group consisting of: Alzheimer's disease, Parkinson's disease, Huntington disease, frontotemporal dementia, amyotrophic lateral sclerosis, multiple sclerosis, glaucoma, myotonic dystrophy, progressive supranuclear palsy, spinal muscular atrophy, multisystem atrophy, ataxias, vascular dementia, or other dementias.

Described herein, in certain embodiments are methods of identifying a subject as having an increased risk of developing microglial dysfunction, the method comprising identifying a subject having an elevated level of CML, a CML precursor, a CML breakdown product, or a CML metabolite in a biological sample obtained from the subject as compared to a reference level, wherein such elevated level is indicative the subject has an increased risk of developing microglial dysfunction.

Described herein, in certain embodiments are methods of identifying a subject as having an increased risk of cognitive impairment, the method comprising identifying a subject having an elevated level of CML, a CML precursor, a CML breakdown product, or a CML metabolite in a biological sample obtained from the subject as compared to a reference level, wherein such elevated level is indicative the subject has an increased risk of developing cognitive impairment.

Described herein, in certain embodiments are methods of identifying a subject as having an increased risk of developing a neurodegenerative disease, the method comprising identifying a subject having an elevated level of CML, a CML precursor, a CML breakdown product, or a CML metabolite in a biological sample obtained from the subject as compared to a reference level, wherein such elevated level is indicative the subject has an increased risk of developing neurodegenerative disease.

Described herein, in certain embodiments are methods of screening for a candidate agent for decreasing the rate of accumulation of CML, a CML precursor, a CML breakdown product, or a CML metabolite in a tissue or body fluid sample of a subject, the method comprising: determining a first level of CML, a CML precursor, a CML breakdown product, or a CML metabolite in a biological sample obtained in a mammal at a first time point; administering an agent to the subject; and determining a second level of CML, the CML precursor, the CML breakdown product, or the CML metabolite in a biological sample obtained in a mammal at a second time point; wherein an agent that results in a reduction in the second level as compared to the first level is identified as a candidate agent for decreasing the rate of accumulation of CML, a CML precursor, a CML breakdown product, or a CML metabolite in a tissue of a subject.

Described herein, in certain embodiments are methods of screening for a candidate agent for decreasing the rate of development of oxidative or metabolic stress in microglia in a subject, the method comprising: determining a first level of CML, a CML precursor, a CML breakdown product, or a CML metabolite in a biological sample obtained in a mammal at a first time point; administering an agent to the subject; and determining a second level of CML, the CML precursor, the CML breakdown product, or the CML metabolite in a biological sample obtained in a mammal at a second time point; wherein an agent that results in a reduction in the second level as compared to the first level is identified as a candidate agent for decreasing the rate of development of oxidative or metabolic stress in microglia in a subject.

Described herein, in certain embodiments are methods of screening for a candidate agent for decreasing the rate of development of oxidative or metabolic stress in microglia in a subject, the method comprising: determining a first level of CML, a CML precursor, a CML breakdown product, or a CML metabolite in a biological sample obtained in a mammal at a first time point; administering an agent to the subject; and determining a second level of CML, the CML precursor, the CML breakdown product, or the CML metabolite in a biological sample obtained in a mammal at a second time point; wherein an agent that results in a reduction in the second level as compared to the first level is identified as a candidate agent for decreasing the rate of development of oxidative or metabolic stress in microglia in a subject.

Described herein, in certain embodiments are methods of screening for a candidate agent for decreasing the rate of development of mitochondrial dysfunction in microglia in a subject, the method comprising: determining a first level of CML, a CML precursor, a CML breakdown product, or a CML metabolite in a biological sample obtained in a mammal at a first time point; administering an agent to the subject; and determining a second level of CML, the CML precursor, the CML breakdown product, or the CML metabolite in a biological sample obtained in a mammal at a second time point; wherein an agent that results in a reduction in the second level as compared to the first level is identified as a candidate agent for decreasing the rate of development of mitochondrial dysfunction in microglia in a subject.

Described herein, in certain embodiments are methods of screening for a candidate agent for decreasing the rate of development of microglial dysfunction in a subject, the method comprising: determining a first level of CML, a CML precursor, a CML breakdown product, or a CML metabolite in a biological sample obtained in a mammal at a first time point; administering an agent to the subject; and determining a second level of CML, the CML precursor, the CML breakdown product, or the CML metabolite in a biological sample obtained in a mammal at a second time point; wherein an agent that results in a reduction in the second level as compared to the first level is identified as a candidate agent for decreasing the rate of development of microglial dysfunction in a subject.

Described herein, in certain embodiments are methods of screening for a candidate agent for increasing one or more activities of microglia in a subject, the method comprising: determining a first level of CML, a CML precursor, a CML breakdown product, or a CML metabolite in a biological sample obtained in a mammal at a first time point; administering an agent to the subject; and determining a second level of CML, the CML precursor, the CML breakdown product, or the CML metabolite in a biological sample obtained in a mammal at a second time point; wherein an agent that results in a reduction in the second level as compared to the first level is identified as a candidate agent for increasing one or more activities of microglia in a subject.

Described herein, in certain embodiments are methods of screening for a candidate agent for decreasing the rate of development or worsening of cognitive impairment in a subject, the method comprising: determining a first level of CML, a CML precursor, a CML breakdown product, or a CML metabolite in a biological sample obtained in a mammal at a first time point; administering an agent to the subject; and determining a second level of CML, the CML precursor, the CML breakdown product, or the CML metabolite in a biological sample obtained in a mammal at a second time point; wherein an agent that results in a reduction in the second level as compared to the first level is identified as a candidate agent for decreasing the rate of development or worsening of cognitive impairment in a subject.

Described herein, in certain embodiments are methods of screening for a candidate agent for treating cognitive impairment in a subject, the method comprising: determining a first level of CML, a CML precursor, a CML breakdown product, or a CML metabolite in a biological sample obtained in a mammal at a first time point; administering an agent to the subject; and determining a second level of CML, the CML precursor, the CML breakdown product, or the CML metabolite in a biological sample obtained in a mammal at a second time point; wherein an agent that results in a reduction in the second level as compared to the first level is identified as a candidate agent for treating cognitive impairment in a subject.

Described herein, in certain embodiments are methods of screening for a candidate agent for decreasing the rate of development or progression of a neurodegenerative disease in a subject, the method comprising: determining a first level of CML, a CML precursor, a CML breakdown product, or a CML metabolite in a biological sample obtained in a mammal at a first time point; administering an agent to the subject; and determining a second level of CML, the CML precursor, the CML breakdown product, or the CML metabolite in a biological sample obtained in a mammal at a second time point; wherein an agent that results in a reduction in the second level as compared to the first level is identified as a candidate agent for decreasing the rate of development or progression of a neurodegenerative disease in a subject.

Described herein, in certain embodiments are methods of screening for a candidate agent for treating a neurodegenerative disease in a subject, the method comprising: determining a first level of CML, a CML precursor, a CML breakdown product, or a CML metabolite in a biological sample obtained in a mammal at a first time point; administering an agent to the subject; and determining a second level of CML, the CML precursor, the CML breakdown product, or the CML metabolite in a biological sample obtained in a mammal at a second time point; wherein an agent that results in a reduction in the second level as compared to the first level is identified as a candidate agent for treating a neurodegenerative disease in a subject.

Described herein, in certain embodiments are methods of screening for a candidate agent for decreasing the rate of development or worsening of neuronal dysfunction in a subject, the method comprising: determining a first level of CML, a CML precursor, a CML breakdown product, or a CML metabolite in a biological sample obtained in a mammal at a first time point; administering an agent to the subject; and determining a second level of CML, the CML precursor, the CML breakdown product, or the CML metabolite in a biological sample obtained in a mammal at a second time point; wherein an agent that results in a reduction in the second level as compared to the first level is identified as a candidate agent for decreasing the rate of development or worsening of neuronal dysfunction in a subject.

In certain embodiments, the method further comprises testing the candidate agent in an animal model.

### INCORPORATION BY REFERENCE

All publications and patents cited throughout the text of this specification (including all patents, patent applications, scientific publications (e.g., Mossad et al. (2022) NATURE NEUROSCIENCE, 25: 295-305), manufacturer's specifications, instructions, etc.), whether supra or infra, are hereby incorporated by reference in their entirety for all purposes. To the extent the material incorporated by reference contradicts or is inconsistent with this specification, the specification will supersede any such material.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of decreasing the rate of development of oxidative stress or mitochondrial dysfunction in microglia, or microglial dysfunction in a subject in need thereof, the method comprising administering to the subject a therapeutically effective amount of a gut barrier function enhancer and/or an agent for reducing or eliminating gut microbiota dysbiosis.

2. A method of preventing or treating cognitive impairment or a neurodegenerative disease, decreasing the rate of development or worsening of cognitive impairment, or decreasing the rate of development or progression of a neurodegenerative disease in a subject in need thereof, the method comprising administering to the subject a therapeutically effective amount of a gut barrier function enhancer and/or an agent for reducing or eliminating gut microbiota dysbiosis.

3. (canceled)

4. The method of claim 2, wherein the method decreases neuronal dysfunction.

5. The method of claim 1, wherein;
(i) the subject has previously been identified as having an elevated level of N6-carboxymethyllysine (CVL), a CML precursor, a CML metabolite, or a CML analog in a biological sample of the subject as compared to a reference level; and/or
(ii) the method further comprises identifying the subject as having an elevated level of CML, a CML precursor, a CML metabolite, or a CML analog in a biological sample of the subject as compared to a reference level.

6. The method of claim 1, wherein:
(i) the subject has previously been identified as having an elevated level of permeability of the gut barrier as compared to a reference level; and/or
(ii) the method further comprises identifying the subject as having an elevated level of permeability of the gut barrier as compared to a reference level.

7. The method of claim 5, wherein the biological sample comprises a body fluid or tissue sample.

8. The method of claim 1, wherein the subject has been identified or diagnosed as having:
(i) a cognitive impairment; and/or
(ii) a neurodegenerative disease.

9. The method of claim 1, wherein the subject has been identified as having an increased risk of developing:
(i) a cognitive impairment; and/or
(ii) a neurodegenerative disease.

10. The method of claim 1, wherein the method results in a reduction in a level of cellular and/or mitochondrial reactive oxidative species (ROS) in microglia in the subject.

11. The method of claim 1, wherein the method results in:
(a) a reduction in expression of inducible nitric oxide synthase (iNOS) in microglia in the subject;
(b) a reduction in expression of one or more genes in microglia of the subject selected from the group consisting of Cdkn1a, Cyba, Cybb, Duoxa1, Il1b, Tgfbr2, Tir2, Tir4, TirS, Axl, Hif1a, Lcn2, Mmp2, Rela, Trex1, S100a8, and S100a9; and/or
(c) an increase in expression of one or more genes in microglia of the subject selected from the group consisting of Foxp1, Nrf1, Trp53, G6pdx, Pdk2, Stat3, and Ucp2.

12-13. (canceled)

14. The method of claim 5, further comprising determining the level of CML, a CML precursor, a CML metabolite, or a CML analog in the biological sample obtained from the subject.

15. The method of claim 8 (ii), wherein the neurodegenerative disease is selected from the group consisting of: Alzheimer's disease, Parkinson's disease, Huntington disease, frontotemporal dementia, amyotrophic lateral sclerosis, multiple sclerosis, glaucoma, myotonic dystrophy, progressive supranuclear palsy, spinal muscular atrophy, multisystem atrophy, ataxias, and vascular dementia.

16-18. (canceled)

19. The method of claim 2, wherein the method:
(a) reduces a concentration of CVL, a CML precursor, a CML metabolite, or a CML analog in a blood or a brain sample;
(b) decreases the rate of accumulation of CVL, a CML precursor, a CML metabolite, or a CML analog in a tissue of a subject; and/or
(c) reduces gut permeability.

20. (canceled)

21. The method of claim 1, wherein the gut barrier function enhancer and/or an agent for reducing or eliminating gut microbiota dysbiosis comprises intestinal alkaline phosphatase (IAP), lipoteichoic acid, metformin, ellagic acid (EA), urolithin A, butyrate, glutamine, obeticholic acid (OCA), divertin, or curcumin, or derivatives thereof.

22. The method of claim 21, wherein the gut barrier function enhancer and/or an agent for reducing or eliminating gut microbiota dysbiosis is IAP.

23. The method of claim 21, wherein the gut barrier function enhancer and/or an agent for reducing or eliminating gut microbiota dysbiosis is EA.

24. The method of claim 1, wherein the gut barrier function enhancer and/or an agent for reducing or eliminating gut microbiota dysbiosis is formulated as a pharmaceutical composition comprising IAP, lipoteichoic acid, metformin, EA, urolithin A, butyrate, glutamine, OCA, divertin, or curcumin, or a pharmaceutically acceptable salt thereof.

25. The method of claim 1, wherein the gut barrier function enhancer and/or an agent for reducing or eliminating gut microbiota dysbiosis is administered by oral administration, transdermal administration, inhalation, nasal administration, topical administration, intravenous administration, intra-arterial administration, intramuscular administration, or subcutaneous administration.

26. The method of claim 1, wherein following administration of the gut barrier function enhancer and/or an agent for reducing or eliminating gut microbiota dysbiosis, the subject exhibits one or more of:
(a) a reduced concentration of CML, a CML precursor, a CML metabolite, or a CML analog in a blood sample;
(b) a reduced concentration of CML, a CML precursor, a CML metabolite, or a CML analog in a brain tissue sample;
(c) a reduced gut permeability;
(d) a reduction of microbiota dysbiosis;
(e) an increased level of autophagy in gut epithelium;
(f) a reduction in level of cellular and/or mitochondrial ROS in microglia;
(g) an increased level of adenosine triphosphate (ATP) in a population of microglia;
(h) a reduction in expression of iNOS in microglia;
(i) a reduction in expression of one or more genes in microglia selected from the group consisting of Cdkn1a, Cyba, Cybb, Duoxa1, Il1b, Tgfbr2, Tir2, Tir4, TirS, Axl, Hif1a, Lcn2, Mmp2, Rela, Trex1, S100a8, and S00a9; and
(j) an increase in expression of one or more genes in microglia selected from the group consisting of Foxp1, Nrf1, Trp53, G6pdx, Pdk2, Stat3, and Ucp2.

27-33. (canceled)

34. The method of claim 7, wherein:
(a) the body fluid is selected from the group consisting of saliva, urine, blood, serum, plasma, cerebrospinal fluid, and feces; and/or
(b) the tissue sample is a brain tissue.
